(19) United States
(12) Patent Application Publication
(10) Pub. No.: US 2026/0217836 A1
(43) Pub. Date: Jul. 30, 2026

(54) COMBINATION TREATMENT OF CANCERS USING AN ANTIBODY THAT BINDS AT LEAST EGFR AND AN IMMUNE CHECKPOINT INHIBITOR

(71) Applicant: Merus N.V., Utrecht (NL)

(72) Inventors: Ernesto Isaac WASSERMAN, Utrecht (NL); Eduardo Jose PENNELLA, Utrecht (NL)

(21) Appl. No.: 19/142,031

(22) PCT Filed: Dec. 22, 2023
(86) PCT No.: PCT/NL2023/050684

§ 371 Date: Jun. 20, 2025

(30) Foreign Application Priority Data
Dec. 23, 2022 (NL) 2033821

(51) Int. Cl. C07K 16/28; A61P 35/00; C07K 16/30

(52) U.S. Cl. CPC C07K 16/2863; A61P 35/00; C07K 16/2818; C07K 16/2827; C07K 16/30; C07K 2317/24; C07K 2317/31

(57) ABSTRACT

The disclosure relates to means and methods in the treatment of cancer. The disclosure in particular relates to a method of treating a cancer in an individual with an antibody that at least binds EGFR and an immune checkpoint inhibitor (ICI). The invention further relates to the use in such methods and to use in the manufacture of a medicament for the treatment of a cancer that has not been previously treated with a therapeutic agent having anti-cancer properties. Such a combination of said antibodies and an ICI are particularly useful in the treatment of cancers, such as head and neck cancer.

### FIELD OF THE INVENTION

The disclosure relates to means and methods in the treatment of cancer. The disclosure in particular relates to a method of treating a cancer in an individual with an antibody that at least binds extracellular EGFR monovalently. The invention further relates to the use of said antibody in such methods and its use in the manufacture of a medicament for the treatment of a head and neck cancer. Such antibodies are particularly useful in the treatment of cancers such as head and neck cancer.

### BACKGROUND OF THE INVENTION

Cancer is still a major cause of death in the world, in spite of the many advances that have been made in the treatment of the disease and the increased knowledge of the molecular events that lead to cancer.

Traditionally, most cancer drug discovery has focused on agents that block essential cell functions and kill dividing cells. However, in cases of advanced cancer, no matter how aggressively applied, even to the point where patients suffer life-threatening side-effects from the treatment, chemotherapy rarely results in a complete cure. In most cases, the tumors in the patients stop growing or temporarily shrink (referred to as remission) only to start proliferating again, sometimes more rapidly (referred to as relapse), and become increasingly more difficult to treat. More recently the focus of cancer drug development has moved away from broadly cytotoxic chemotherapy to targeted cytostatic therapies with less toxicity. Treatment of advanced cancer has been validated clinically in leukemia and some other cancers. However, in a majority of carcinomas, targeted approaches are still proving not to be effective enough with significant unmet medical need existing, and leaving a need for safer and more effective treatment options.

Targeting of cancers has been achieved using a variety of different methods including for instance small molecules directed towards signaling proteins on which the cancer depends for survival and/or growth; vaccines with tumor specific proteins; cell therapies with immune cells that actively kill tumor cells, and antibodies that target cytotoxic molecules to the tumor; interfere with signaling and/or that (re)direct the immune system of the host to the tumor cells.

It has been reported that, in the United States, head and neck cancer, in particular in the oral cavity and pharynx, already accounts for 3 percent of malignancies, with approximately 53,000 Americans developing such cancer annually and 10,800 dying therefrom (Siegel et al., CA Cancer J Clin. 2020; 70(1):7. Epub 2020 Jan. 8.).

Furthermore, head and neck squamous cell carcinoma (HNSCC) is reported to be the sixth leading cancer by incidence worldwide, with a five-year overall survival rate of patients with HNSCC of about 40-50% (in Head and Neck Cancer, Union for International Cancer Control, 2014 Review of Cancer Medicines on the WHO List of Essential Medicines).

In the KEYNOTE-048 study (Burtness et al., Vol. 394, 10212, pages 1915-1928, Nov. 23, 2019, the Lancet), pembrolizumab was used as monotherapy at 200 mg IV Q3W for a maximum of 24 months. In patients with a CPS≥1, the mean OS was 12.3 months (95% confidence interval [CI]: 10.8, 14.9); the mean PFS was 3.2 months (95% CI: 2.2, 3.4); the best objective response as confirmed complete response (CR) or PR was merely 19% (CI: 14.5, 24.4); and the mean duration of response (DOR) was 20.9 months (95% CI: 1.5, 34.8).

A recent phase 2 study combining pembrolizumab with cetuximab in a population of HNSCC patients who had not received anti-PD1 or EGFR therapies previously achieved an objective response rate (ORR) by 6 months in 15 of 33 participants (45% [95% CI: 28,62]); the mean DOR was 13.1 months (95% CI 6.5, not reached), with 1 patient achieving a response after 6 months, thereby elevating the ORR to 48%. The mean PFS was 6.5 months (95% CI: 2.1, not reached), and the mean OS was 18.4 months (95% CI: 11.0, not reached) (Sacco et al., 2021, Lancet Oncol. June; 22(6):883-892). In that phase 2 combination study, the observed adverse events (AEs) were as expected, based on each drug's individual toxicity profile. Fatigue, cutaneous events (rash and dry skin), hypomagnesemia and transaminitis were among the most reported TEAEs. Immune-related AEs related to pembrolizumab were consistent with its toxicity profile. A potential overlapping toxicity was observed as oral mucositis and skin toxicity.

A meta-analysis into locoregionally advanced head and neck squamous cell carcinoma (LA-HNSCC) reported that the addition of an anti-EGFR agent to radiotherapy or chemoradiotherapy did not improve clinical outcomes in patients with LA-HNSCC (Oncotarget. 2017; 8(60):102371-102380). Also, the addition of anti-EGFR agents was reported to increase the risk of skin toxicities and mucositis.

A need thus exists for improved cancer treatments, in particular for treatment of head and neck cancer.

### SUMMARY OF THE INVENTION

The disclosure provides the following preferred aspects. However, the invention is not limited thereto.

The present disclosure relates to a combination therapy, wherein an antibody or functional part, derivative and/or analogue thereof that comprises an antigen binding site that binds an extracellular part of EGFR is used with an immune checkpoint inhibitor in a method of treatment of cancer in a subject in need thereof.

The present disclosure provides means and methods for (re)directing immune system components in the treatment of cancer.

Also, the present disclosure relates to an antibody or functional part, derivative and/or analogue thereof that comprises a variable domain that binds an extracellular part of EGFR for use in the treatment of a cancer in a subject, wherein the treatment further comprises administering an immune checkpoint inhibitor.

Also, the present disclosure relates to a use of an antibody or functional part, derivative and/or analogue thereof that comprises a variable domain that binds an extracellular part of EGFR and an immune checkpoint inhibitor in the manufacture of one or more medicaments for treating a cancer in a subject. In certain aspects, the antibody or functional part, derivative and/or analogue thereof and the immune checkpoint inhibitor are used to manufacture separate medicaments. In certain aspects, the treatment of cancer comprises administration of the antibody or functional part, derivative and/or analogue thereof and the immune checkpoint inhibitor.

Also the present disclosure relates to a method of treating cancer in a subject, the method comprising administering an effective amount of an antibody or functional part, derivative and/or analogue thereof that comprises a variable domain that binds an extracellular part of EGFR and an effective amount of an immune checkpoint inhibitor to the subject.

In certain aspects, said immune checkpoint inhibitor comprises a PD-L1, PD-L2 or PD-1 inhibitor. In certain aspects, the immune checkpoint inhibitor comprises or is a PD-L1, PD-L2 or PD-1 inhibitor. In certain aspects, the immune checkpoint inhibitor comprises or is an antibody. In certain aspects, the immune checkpoint inhibitor comprises or is an antibody that targets PD-L1, PD-L2 or PD-1. In certain aspects, the immune checkpoint inhibitor comprises or is an antibody that inhibits PD-L1, PD-L2 or PD-1. In certain aspects, the immune checkpoint inhibitor comprises or is an antibody that inhibits PD-L1. In certain aspects, the immune checkpoint inhibitor comprises or is an antibody that inhibits PD-L2. In certain aspects, the immune checkpoint inhibitor comprises or is an antibody that inhibits PD-1.

In certain aspects, the immune checkpoint inhibitor comprises or is nivolumab, pembrolizumab, cemiplimab, penpulimab, retifanlimab, sintilimab, tislelizumab, toripalimab, dostarlimab, atezolizumab, avelumab or durvalumab.

In certain aspects, the immune checkpoint inhibitor comprises or is pembrolizumab. In certain aspects, the immune checkpoint inhibitor comprises or is nivolumab.

In certain aspects, the cancer comprises or is an adenocarcinoma, a squamous cell carcinoma, or a head and neck cancer, including squamous cell carcinoma of the head and neck (SCCHN).

In certain aspects, the cancer comprises or is a head and neck cancer, including squamous cell carcinoma of the head and neck (SCCHN).

In certain aspects, the antibody or functional part, derivative and/or analogue thereof, comprises or is a multispecific antibody. In certain aspects, the antibody or functional part, derivative and/or analogue thereof, comprises or is a bispecific antibody.

In certain aspects, the antibody or functional part, derivative and/or analogue thereof of the present disclosure is a multispecific antibody that at least binds EGFR. In certain aspects, the antibody or functional part, derivative and/or analogue thereof of the present disclosure is a bispecific antibody that at least binds EGFR. In certain aspects, the antibody binds EGFR monovalently. In certain aspects, the antibody comprises a second variable domain that does not bind EGFR. In certain aspects, the antibody or functional part, derivative and/or analogue thereof, comprises a variable domain that binds LGR5. In certain aspects, the antibody or functional part, derivative and/or analogue thereof is ADCC enhanced. Also, in certain aspects, the antibody or functional part, derivative and/or analogue thereof is afucosylated. In certain aspects, the antibody that binds at least EGFR comprises or is petosemtamab.

In certain aspects, the cancer expresses PD-L1, EGFR and/or LGR5. In certain aspects, the cancer expresses PD-L1 and EGFR.

In certain aspects, the treatment is first line treatment. In certain aspects, the subject receiving the treatment has not received an earlier or a prior anti-cancer treatment for said cancer.

In certain aspects, the antibody that binds at least EGFR is petosemtamab and is administered in a dose of 1500 mg. In certain aspects, petosemtamab is administered in a dose of 1500 mg once every two weeks. In certain aspects, the immune checkpoint inhibitor is pembrolizumab and is administered in a dose of 600 mg. In certain aspects, the immune checkpoint inhibitor is pembrolizumab and is administered in a dose of 600 mg once every six weeks.

In certain aspects, the subject of the present disclosure is a mammalian subject, such as a human subject.

The disclosure further comprises a (pharmaceutical) combination, or a kit-of-parts, that comprises the antibody or functional part, derivative and/or analogue thereof that binds at least EGFR of the present disclosure combined with the immune checkpoint inhibitor of the present disclosure. Said combination is in certain aspects not physically linked and comprises a container containing said antibody or functional part, derivative and/or analogue thereof that binds at least EGFR, and a container containing said immune checkpoint inhibitor as mentioned herein. Said combination is in certain aspects accompanied by instructions for use. The instructions for use include clinically relevant information, such as instructions for intravenous administration, the dose to be administered and the time interval of administration.

In certain aspects, the antibody or functional part, derivative and/or analogue thereof, in particular petosemtamab, and the immune checkpoint inhibitor, in particular pembrolizumab, will be administered according to the instructions for use following approval of relevant authorities.

### BRIEF DESCRIPTION OF THE DRAWINGS

FIG. 1. Human LGR5 sequence; Sequence ID NO: 1.

FIG. 2. Human EGFR sequence; Sequence ID NO: 2.

FIG. 3. (a) Amino acid sequences of heavy chain variable regions (Sequence ID Nos: 3-15) that together with a common light chain variable region such as the variable region of the human kappa light chain IgVκ1 39*01/IGJκ1*01 form a variable domain that binds LGR5 and EGFR. The CDR and framework regions are indicated in FIG. 3b, following Kabat numbering.

FIG. 4. a) Amino acid sequence of a common light chain amino acid sequence. b) Common light chain variable region (IGKV1-39/jk1). c) Light chain constant region. d) V-region IGKV1-39A; e) CDR1, CDR2 and CDR3 of a common light chain according to IMGT numbering.

FIG. 5. IgG heavy chains for the generation of bispecific molecules. a) CH1 region. b) Hinge region. c) CH2 region. d) CH3 domain containing variations L351K and T366K (KK). e) CH3 domain containing variations L351D and L368E (DE). Residue positions are according to EU numbering.

### DETAILED DESCRIPTION OF THE DISCLOSURE

The present disclosure relates to a combination therapy, wherein an antibody or functional part, derivative and/or analogue thereof that comprises an antigen binding site that binds an extracellular part of EGFR is used with an immune checkpoint inhibitor in a method of treatment of cancer in a subject in need thereof.

The present disclosure relates to an antibody or functional part, derivative and/or analogue thereof that comprises an antigen binding site that binds an extracellular part of EGFR for use in a method of treatment of a cancer in a subject in need thereof, which method further comprises administration of an immune checkpoint inhibitor for treatment of said cancer.

Also, the present disclosure relates to an antibody or functional part, derivative and/or analogue thereof that comprises a variable domain that binds an extracellular part of EGFR for use in a method of treatment of cancer in a subject, wherein the treatment further comprises the use of an immune checkpoint inhibitor.

Also, the present disclosure relates to a method of treating cancer in a subject, the method comprising administering an effective amount of an antibody or functional part, derivative and/or analogue thereof that comprises a variable domain that binds an extracellular part of EGFR and an effective amount of an immune checkpoint inhibitor to the subject.

Also, the present disclosure relates to a use of an antibody or functional part, derivative and/or analogue thereof that comprises a variable domain that binds an extracellular part of EGFR and an immune checkpoint inhibitor in the manufacture of one or more medicaments for treating a cancer in a subject. In certain aspects, the antibody or functional part, derivative and/or analogue thereof and the immune checkpoint inhibitor are used to manufacture separate medicaments, such as two separate medicaments, one for said antibody or functional part, derivative and/or analogue thereof and one for said immune checkpoint inhibitor. Said medicament comprising said antibody, functional part, derivative and/or analogue thereof that comprises a variable domain that binds an extracellular part of EGFR may be contained in a holder that is separate, meaning not physically linked, from a holder that contains said immune checkpoint inhibitor as a medicament. In certain aspects, the treatment of cancer comprises administration of the antibody or functional part, derivative and/or analogue thereof that binds at least EGFR and the immune checkpoint inhibitor.

In certain aspects, the present disclosure provides the use of an antibody or functional part, derivative and/or analogue of the present disclosure that comprises a variable domain that can bind an extracellular part of EGFR and an immune checkpoint inhibitor in the manufacture of a medicament for the treatment of a cancer.

In certain aspects, the present disclosure provides the use of an antibody or functional part, derivative and/or analogue of the present disclosure that comprises a variable domain that can bind an extracellular part of EGFR in the manufacture of a medicament for increasing the effect of an immune checkpoint inhibitor for the treatment of cancer.

In certain aspects, the present disclosure provides the use of an immune checkpoint inhibitor in the manufacture of a medicament for increasing the effect of an antibody or functional part, derivative and/or analogue of the present disclosure that comprises a variable domain that can bind an extracellular part of EGFR for the treatment of cancer.

Also, the present disclosure provides a kit of parts comprising an antibody or functional part, derivative and/or analogue thereof that comprises a variable domain that can bind an extracellular part of EGFR, an immune checkpoint inhibitor and instructions for use of said antibody or functional part, derivative and/or analogue thereof and for use of said immune checkpoint inhibitor.

Also, the present disclosure provides a combination of an antibody or functional part, derivative and/or analogue thereof that comprises a variable domain that can bind an extracellular part of EGFR and an immune checkpoint inhibitor as mentioned herein for use in the treatment of cancer in a subject in need thereof.

Also, the present disclosure provides a combination of an immune checkpoint inhibitor, as mentioned herein, instructions for use of said immune checkpoint inhibitor in the treatment of cancer in a subject, as well as instructions for use in the treatment of said cancer in said subject of an antibody or functional part, derivative and/or analogue thereof that comprises a variable domain that can bind an extracellular part of EGFR, as mentioned herein.

Also, the present disclosure provides a combination of an antibody or functional part, derivative and/or analogue thereof that comprises a variable domain that can bind an extracellular part of EGFR, as mentioned herein, instructions for use of said antibody or functional part, derivative and/or analogue thereof in the treatment of cancer in a subject, as well as instructions for use in the treatment of said cancer in a subject of an immune checkpoint inhibitor as mentioned herein.

In certain aspects, the present disclosure provides a pharmaceutical composition comprising an antibody or functional part, derivative and/or analogue thereof that comprises a variable domain that can bind an extracellular part of EGFR of the present disclosure and instructions for use thereof with an immune checkpoint inhibitor in the treatment of said cancer.

In certain aspects, the present disclosure provides a pharmaceutical composition for the treatment of a cancer, comprising an antibody or functional part, derivative and/or analogue thereof that comprises a variable domain that can bind an extracellular part of EGFR of the present disclosure and a pharmaceutical composition for the treatment of said cancer, comprising an immune checkpoint inhibitor of the present disclosure.

In certain aspects, the present disclosure provides a pharmaceutical composition for use in the treatment of cancer comprising an antibody or functional part, derivative and/or analogue thereof that comprises a variable domain that can bind an extracellular part of EGFR of the present disclosure, wherein the pharmaceutical composition is administered in combination with an immune checkpoint inhibitor of the present disclosure.

In certain aspects, the present disclosure relates to a pharmaceutical composition for the treatment of a cancer comprising an antibody or functional part, derivative and/or analogue thereof that comprises a variable domain that can bind an extracellular part of EGFR of the present disclosure, wherein a subject to be treated is further administered an immune checkpoint inhibitor prior to, simultaneously with, or after administration of said bispecific antibody.

In certain aspects, the present disclosure relates to a pharmaceutical composition for the treatment of a cancer in a subject comprising an immune checkpoint inhibitor, wherein said subject to be treated is further administered an antibody or functional part, derivative and/or analogue thereof that comprises a variable domain that can bind an extracellular part of EGFR of the present disclosure prior to, simultaneously with, or after administration of said immune checkpoint inhibitor.

The present disclosure thus relates to a combination of medicaments for the treatment of cancer in a subject which comprises administration to said subject of multiple, different medicaments for treating said cancer, which treatment comprises simultaneous, sequential or separate administration of said medicaments. In certain aspects, said medicament comprises an antibody or functional part, derivative and/or analogue thereof that comprises a variable domain that can bind an extracellular part of EGFR of the present disclosure, and said other, different medicament comprises an immune checkpoint inhibitor.

In certain aspects, the antibody or functional part, derivative and/or analogue thereof that comprises a variable domain that can bind an extracellular part of EGFR of the present disclosure may be administered simultaneously, sequentially or separately with the immune checkpoint inhibitor of the present disclosure. Said combination of the antibody or functional part, derivative and/or analogue thereof that comprises a variable domain that can bind an extracellular part of EGFR of the present disclosure and immune checkpoint inhibitor thus encompasses simultaneous, sequential or separate administration.

Hence, in certain aspects, the present disclosure provides an antibody or functional part, derivative and/or analogue thereof that comprises a variable domain that can bind an extracellular part of EGFR of the present disclosure for use in a method of treatment of a cancer, wherein the treatment further comprises administering an immune checkpoint inhibitor, wherein optionally said antibody or functional part, derivative and/or analogue thereof is administered simultaneously, sequentially or separately with said immune checkpoint inhibitor.

Hence, in certain aspects, the present disclosure provides a method of treatment of a subject having a cancer, comprising administering to the subject an effective amount of an immune checkpoint inhibitor and an antibody or functional part, derivative and/or analogue thereof that comprises a variable domain that can bind an extracellular part of EGFR of the present disclosure, wherein optionally said antibody or functional part, derivative and/or analogue thereof is administered simultaneously, sequentially or separately with said immune checkpoint inhibitor.

Hence, in certain aspects, the present disclosure provides the use of an antibody or functional part, derivative and/or analogue thereof that comprises a variable domain that can bind an extracellular part of EGFR of the present disclosure and an immune checkpoint inhibitor in the manufacture of a medicament for the treatment of a cancer, wherein optionally said antibody or functional part, derivative and/or analogue thereof is administered simultaneously, sequentially or separately with said immune checkpoint inhibitor. In certain aspects, said antibody or functional part, derivative and/or analogue thereof is administered prior to, simultaneously with, or after administration of said immune checkpoint inhibitor.

In certain aspects, the antibody or functional part, derivative and/or analogue thereof that comprises a variable domain that can bind an extracellular part of EGFR of the present disclosure is for use in the manufacture of a medicament for the treatment of a cancer and the immune checkpoint inhibitor are for use in the manufacture of a medicament for the treatment of said cancer, wherein optionally said antibody or functional part, derivative and/or analogue thereof is administered simultaneously, sequentially or separately with said immune checkpoint inhibitor. Optionally, said antibody or functional part, derivative and/or analogue thereof is administered prior to, simultaneously with, or after administration of said immune checkpoint inhibitor.

In order that the present description may be more readily understood, certain terms are defined here. Additional definitions may be set forth throughout the detailed description where deemed required. Unless separately defined herein, all technical and scientific terms used herein have the same meaning as commonly understood by one of ordinary skill in the art, and conventional methods of immunology, protein chemistry, biochemistry, recombinant DNA techniques and pharmacology are employed.

As used herein, the singular forms “a”, “an” and “the” include plural referents. Use of the term “comprising” “having” “including” as well as other forms, such as “comprise”, “comprises”, “comprised”, “has”, “have”, “had”, “include”, “includes”, and “included”, is not limiting.

The term “antibody” as used herein means a proteinaceous molecule belonging to the immunoglobulin class of proteins, containing one or more domains that bind an epitope on an antigen, where such domains are or derived from or share sequence homology with the variable region of an antibody. Antibodies are typically made of basic structural units, each with two heavy chains and two light chains. An antibody according to the present invention is not limited to any particular format or method of producing it.

A “bispecific antibody” is an antibody as described herein wherein one domain of the antibody binds to one antigen whereas a further domain of the antibody binds to a further antigen, wherein said one and further antigens are not identical, or where one domain binds one epitope on an antigen, whereas a further domain binds to a further epitope on the antigen. The term “bispecific antibody” also encompasses antibodies wherein one heavy chain variable region/light chain variable region (VH/VL) combination binds an antigen or epitope on an antigen and a further VH/VL combination that binds a further antigen or epitope on the antigen. The term further includes antibodies wherein VH is capable of specifically recognizing one antigen and the VL, paired with the VH in an immunoglobulin variable region, is capable of specifically recognizing a further antigen. The resulting VH/VL pair will bind either antigen 1 or antigen 2. Such so called “two-in-one antibodies”, described in for instance WO 2008/027236, WO 2010/108127 and Schaefer et al (Cancer Cell 20, 472-486, October 2011). A bispecific antibody according to the present invention is not limited to any particular bispecific format or method of producing it.

The term ‘common light chain’ as used herein refers to the two light chains (or the VL part thereof) in the bispecific antibody. The two light chains (or the VL part thereof) may be identical or have some amino acid sequence differences while the binding specificity of the full-length antibody is not affected. The terms ‘common light chain’, ‘common VL’, ‘single light chain’, ‘single VL’, with or without the addition of the term ‘rearranged’ are all used herein interchangeably. “Common” also refers to functional equivalents of the light chain of which the amino acid sequence is not identical. Many variants of said light chain exist wherein mutations (deletions, substitutions, insertions and/or additions) are present that do not influence the formation of functional binding regions. In certain aspects, the light chain of the present invention can also be a light chain as specified herein, having from 0 to 10 amino acid insertions, deletions, substitutions, additions or a combination thereof. In certain aspects, said 0 to 10 amino acid insertions, deletions, substitutions, additions or a combination thereof are not within the CDR regions. In certain aspects, the light chain of the present invention can also be a light chain as specified herein, having from 0 to 5 amino acid insertions, deletions, substitutions, additions or a combination thereof. It is for instance within the scope of the definition of common light chains as used herein, to prepare or find light chains that are not identical but still functionally equivalent, e.g., by introducing and testing conservative amino acid changes, changes of amino acids in regions that do not or only partly contribute to binding specificity when paired with the heavy chain, and the like.

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, said additional component(s) not altering the unique characteristic of the invention.

A “derivative of an antibody” is a protein that but for the CDR regions deviates from the amino acid sequence of a natural antibody in at most 20 amino acids. A derivative of an antibody as disclosed herein is an antibody that deviates from said amino acid sequence in at most 20 amino acids. The functional part, derivative and/or analogue maintains the binding specificity of the (bispecific) antibody. An “analogue of an antibody” is a protein that may be different in structure, format or origin but maintains the binding specificity of the antibody it is an analogue of.

“Percent (%) identity” as referring to nucleic acid or amino acid sequences herein is defined as the percentage of residues in a candidate sequence that are identical with the residues in a selected sequence, after aligning the sequences for optimal comparison purposes. The percent sequence identity comparing nucleic acid sequences is determined using the AlignX application of the Vector NTI Advance® 11.5.2 software using the default settings, which employ a modified ClustalW algorithm (Thompson, J. D., Higgins, D. G., and Gibson T. J., (1994) Nuc. Acid Res. 22(22): 4673-4680), the swgapdnamt score matrix, a gap opening penalty of 15 and a gap extension penalty of 6.66. Amino acid sequences are aligned with the AlignX application of the Vector NTI Advance® 11.5.2 software using default settings, which employ a modified ClustalW algorithm (Thompson, J. D., Higgins, D. G., and Gibson T. J., (1994) Nuc. Acid Res. 22(22): 4673-4680), the blosum62mt2 score matrix, a gap opening penalty of 10 and a gap extension penalty of 0.1.

As an antibody typically recognizes an epitope of an antigen, and such an epitope may be present in other compounds as well, antibodies according to the present invention that “specifically recognize” an antigen, for example, PD-L1, EGFR or LGR5, may recognize other compounds as well, if such other compounds contain the same kind of epitope. Hence, the terms “specifically recognizes” with respect to an antigen and antibody interaction does not exclude binding of the antibodies to other compounds that contain the same kind of epitope.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein (so-called linear and conformational epitopes). Epitopes formed from contiguous, linear amino acids are typically retained on exposure to denaturing solvents, whereas epitopes formed by tertiary folding, conformation are typically lost on treatment with denaturing solvents. An epitope may typically include 3, 4, 5, 6, 7, 8, 9, 10, 11, 12, 13, 14 or 15 amino acids in a unique spatial conformation.

As used herein, the terms “subject” and “patient” are used interchangeably and refer to a mammal such as a human, mouse, rat, hamster, guinea pig, rabbit, cat, dog, monkey, cow, horse, pig and the like (e.g., a patient, such as a human patient, having cancer).

The terms “treat,” “treating,” and “treatment,” as used herein, refer to any type of intervention or process performed on, or administering an active agent or combination of active agents to the subject with the objective of reversing, alleviating, ameliorating, inhibiting, or slowing down or preventing the progression, development, severity or recurrence of a symptom, complication, condition or biochemical indicia associated with a disease.

As used herein, “effective treatment” or “positive therapeutic response” refers to a treatment producing a beneficial effect, e.g., amelioration of at least one symptom of a disease or disorder, e.g., cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, decrease in tumor size, decrease the presence of circulating tumor cells, reduce or prevent metastases of a tumor, slow or arrest tumor growth and/or prevent or delay tumor recurrence or relapse.

The term “effective amount” or “therapeutically effective amount” refers to an amount of an agent or combination of agents that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In terms of tumor development, an effective amount is an amount sufficient to delay tumor development. In terms of tumor recurrence, an effective amount is an amount sufficient to prevent or delay tumor recurrence. An effective amount can be administered in one or more administrations. The effective amount of the agent or composition may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer. In one aspect, an “effective amount” is the amount of an antibody as disclosed herein as the therapeutic agent to affect a decrease in a cancer (for example a decrease in the number of cancer cells); slowing of progression of a cancer or prevent regrowth or recurrence of the cancer. The antibody or functional part, derivative and/or analogue thereof that binds EGFR or binds EGFR and LGR5 of the present disclosure, is also referred herein to as a “therapeutic agent”. In certain aspects, the effective amount of petosemtamab herein is a flat dose of 1500 mg administered on a biweekly basis to a subject having a cancer of the present disclosure. Also, the immune checkpoint inhibitor is herein referred to as a “therapeutic agent”. In certain aspects, the effective amount of the immune checkpoint inhibitor herein is a flat dose of 400 mg pembrolizumab administered once every six weeks to a subject having a cancer of the present disclosure. In certain aspects, the effective amount of the immune checkpoint inhibitor herein is a flat dose of 200 mg pembrolizumab administered once every three weeks to a subject having a cancer of the present disclosure.

As used herein, the term ‘cancer’ applies equally to the term ‘tumor’, such that treatment of a tumor also applies to treatment of cancer.

The term “flat dose” herein refers to a dosing regimen wherein a subject is administered with a fixed amount of a therapeutic substance over multiple administrations, independent of body weight of the subject. Flat dosing is typically abbreviated with qnw, wherein n is an integer indicating the interval and w is week. For instance, a q2w flat dose administration regimen of 1500 mg antibody means a fixed amount of 1500 mg antibody is administered every two weeks. Herein, in certain aspects, the therapeutic substance is an antibody binding EGFR or EGFR and LGR5 that is administered with a q2w dosing regimen of 1500 mg. In certain aspects, administration to said subject comprises at least three q2w flat dosages of 1500 mg. In certain aspects, said administration comprises at least four dosages or more and may last until the patient shows sufficient clinical or radiological progression. In certain aspects, the therapeutic substance is pembrolizumab and is administered with a q6w dosing regimen of 400 mg as the effective amount. In certain aspects, administration to said subject comprises at least three q6w flat dosages of 400 mg. In certain aspects, said administration comprises at least four dosages or more and may last until the subject shows sufficient clinical or radiological progression.

The term “H-score”, sometimes referred to as “histo” score in the art, refers to a reproducible and standardized scoring methodology which can be used to semi-quantitatively calculate expression of a gene of interest in a tumor sample following a protocol based on methods of immunohistochemistry (IHC) or in situ hybridization techniques (ISH), all well-known with the skilled person and follow the ASCO Apr. 10, 2015 posting how to calculate H-scores. See also Hirsch F R, Varella-Garcia M, Bunn P A Jr, et al: Epidermal growth factor receptor in non-small-cell lung carcinomas: Correlation between gene copy number and protein expression and impact on prognosis. J Clin Oncol 21:3798-3807, 2003; and John T, Liu G, Tsao M-S: Overview of molecular testing in non-small-cell lung cancer: Mutational analysis, gene copy number, protein expression and other biomarkers of EGFR for the prediction of response to tyrosine kinase inhibitors. Oncogene 28:S14-S23, 2009. The relevant teachings of these references are herein incorporated by reference.

In the context of H scoring for EGFR, the term “determined using IHC” refers to a method that uses or comprises IHC as the basis for subsequently determining the H score, as opposed to methods alternative to IHC.

Also provided in the present disclosure is an antibody or functional part, derivative and/or analogue thereof that comprises a variable domain that binds an extracellular part of EGFR and an immune checkpoint inhibitor for use in the treatment of a cancer in a subject, wherein said cancer expresses EGFR which is characterized by an IHC score of 3+ and wherein said variable domain comprises the amino acids as disclosed further herein.

Also provided in the present disclosure is an antibody or functional part, derivative and/or analogue thereof that comprises a variable domain that binds an extracellular part of EGFR and an immune checkpoint inhibitor for use in the treatment of a cancer in a subject, wherein said cancer expresses EGFR which is characterized by an H score for EGFR of more than 200 and wherein said variable domain comprises the amino acids as disclosed further herein.

Also provided in the present disclosure is a method of treating a subject having an EGFR expressing cancer, wherein said subject has not received any prior anti-cancer treatment, the method comprising providing the subject with an effective amount of an antibody or functional part, derivative and/or analogue thereof that comprises a variable domain that binds an extracellular part of EGFR and an immune checkpoint inhibitor.

Also provided in the present disclosure is an antibody or functional part, derivative and/or analogue thereof that comprises a variable domain that binds an extracellular part of EGFR and an immune checkpoint inhibitor for use in the treatment of head and neck cancer in a subject, wherein said cancer expresses EGFR which is characterized by an IHC score of 3+.

### EGFR, LGR5, PD-L1/2 and PD-1 as Targets of the Present Disclosure

Epidermal growth factor (EGF) receptor (EGFR, ErbB1, or HER1) is a member of a family of four receptor tyrosine kinases (RTKs), named Her- or cErbB-1, -2, -3 and -4. EGFR is known under various synonyms, the most common of which is EGFR. EGFR has an extracellular domain (ECD) that is composed of four sub-domains, two of which are involved in ligand binding and two of which are involved in homo-dimerisation and hetero-dimerisation. EGFR integrates extracellular signals from a variety of ligands to yield diverse intracellular responses. A major signal transduction pathway activated by EGFR is composed of the Ras-mitogen-activated protein kinase (MAPK) mitogenic signaling cascade. Activation of this pathway is initiated by the recruitment of Grb2 to tyrosine phosphorylated EGFR. This leads to activation of Ras through the Grb2-bound Ras-guanine nucleotide exchange factor Son of Sevenless (SOS). In addition, the PI3-kinase-Akt signal transduction pathway is also activated by EGFR, although this activation is much stronger in case there is co-expression of ErbB-3 (HER3). The EGFR is implicated in several human epithelial malignancies, notably cancers of the breast, bladder, non-small cell lung cancer lung, colon, ovarian head and neck and brain. Activating mutations in the gene have been found, as well as over-expression of the receptor and of its ligands, giving rise to autocrine activation loops. This RTK has therefore been extensively used as target for cancer therapy. Both small-molecule inhibitors targeting the RTK and monoclonal antibodies (mAbs) directed to the extracellular ligand-binding domains have been developed and have shown hitherto several clinical successes, albeit mostly for a select group of patients. The database accession number for the human EGFR protein and the gene encoding it is GenBank NM_005228.3. This accession number is primarily given to provide a further method of identification of EGFR protein as a target, the actual sequence of the EGFR protein bound by an antibody may vary, for instance because of a mutation in the encoding gene such as those occurring in some cancers or the like.

Where reference herein is made to EGFR, the reference refers to human EGFR unless otherwise stated. The variable domain antigen-binding site that binds EGFR, binds EGFR and a variety of variants thereof such as those expressed on some EGFR positive tumors.

The term “LGR” refers to the family of proteins known as Leucine-rich repeat-containing G-protein coupled receptors. Several members of the family are known to be involved in the WNT signaling pathway, of note LGR4; LGR5 and LGR6.

LGR5 is Leucine-Rich Repeat Containing G Protein-Coupled Receptor 5. Alternative names for the gene or protein are Leucine-Rich Repeat Containing G Protein-Coupled Receptor 5; Leucine-Rich Repeat-Containing G Protein-Coupled Receptor 5; G-Protein Coupled Receptor HG38; G-Protein Coupled Receptor 49; G-Protein Coupled Receptor 67; GPR67; GPR49; Orphan G Protein-Coupled Receptor HG38; G Protein-Coupled Receptor 49; GPR49; HG38 and FEX. A protein or antibody of the invention that binds LGR5, binds human LGR5. The LGR5 binding protein or antibody of the invention may, due to sequence and tertiary structure similarity between human and other mammalian orthologs, also bind such an ortholog but not necessarily so. Database accession numbers for the human LGR5 protein and the gene encoding it are (NC_000012.12; NT_029419.13; NC_018923.2; NP_001264155.1; NP_001264156.1; NP_003658.1). The accession numbers are primarily given to provide a further method of identification of LGR5 as a target, the actual sequence of the LGR5 protein bound may vary, for instance because of a mutation in the encoding gene such as those occurring in some cancers or the like. Where reference herein is made to LGR5, the reference refers to human LGR5 unless otherwise stated. The LGR5 antigen binding site binds LGR5 and a variety of variants thereof, such as those expressed by some LGR5 positive tumor cells.

Programmed death-ligand 1 (PD-L1, CD274 or B7 homolog 1 (B7-H1); HGNC: 17635; NCBI Entrez Gene: 29126; UniProtKB/Swiss-Prot: Q9NZQ7) is a protein that in humans is encoded by the CD274 gene. This gene encodes an immune inhibitory receptor ligand that is expressed by hematopoietic and non-hematopoietic cells, such as T cells and B cells and various types of tumor cells. The encoded protein is a type I transmembrane protein that has immunoglobulin V-like and C-like domains. Interaction of this ligand with its receptor inhibits T-cell activation and cytokine production. During infection or inflammation of normal tissue, this interaction is important for preventing autoimmunity by maintaining homeostasis of the immune response. In tumor microenvironments, this interaction provides an immune escape for tumor cells through cytotoxic T-cell inactivation.

PD-L1 is a type 1 transmembrane protein that plays a role in suppressing an immune response during particular events such as pregnancy, tissue allografts, autoimmune disease and other disease states such as hepatitis. PD-L1 is expressed in various types of cancers, especially in NSCLC (Boland et al., 2013. Tumor B7-H1 and B7-H3 expression in Squamous cell carcinoma of the lung. Clinical lung cancer Vol. 14, No. 2, 157-63; Velcheti et al., 2014. Programmed death ligand-1 expression in non-small cell lung cancer. Laboratory investigation. 94, 107-116), melanoma, renal cell carcinoma, gastric cancer, hepatocellular as well as various leukemias and multiple myeloma (Bernstein et al., 2014. Radiation-Induced modulation of costimulatory and coinhibitory T-cell signaling molecules on human prostate carcinoma cells promotes productive antitumor immune interactions. Cancer biotherapy and radiopharmaceuticals. Vol. 29. No. 4, 153-161; Thompson et al., 2005. Costimulatory molecule B7-H1 in primary and metastatic clear cell renal cell carcinoma. Cancer 104:2084-91). PD-L1 is present in the cytoplasm and plasma membrane of cancer cells, but not all cancers or all cells within a tumor express PD-L1 (Dong et al., 2002. Tumor-associated B7-H1 promotes T-cell apoptosis: A potential mechanism of immune invasion. Nature medicine. Vol 8 NO 8 793-800). Multiple tumor microenvironment cells contribute to immune suppression by upregulating PD-L1 expression. This effect is called “adaptive immune resistance”, because the tumor protects itself by inducing PD-L1 in response to IFN-γ produced by activated T cells (Sharma et al., 2017. Primary, adaptive, and acquired resistance to cancer immunotherapy. Cell. Vol. 168. 707-723). PD-L1 can also be regulated by oncogenes, this mechanism is known as inherent immune resistance (Akbay et al., 2013. Activation of the PD-1 pathway contributes to immune excape in EGFR-driven lung tumors. Cancer discovery. 1355-1363). Within the tumor microenvironment, PD-L1 is also expressed on myeloid cells and activated T cells (Tumeh et al., 2014. PD-1 blockade induces responses by inhibiting adaptive immune resistance. Nature. 515(7528): 568-571). The expression of PD-L1 is induced by multiple proinflammatory molecules, including types I and II IFN-γ, TNF-α, LPS, GM-CSF and VEGF, as well as the cytokines IL-10 and IL-4, with IFN-γ being the most potent inducer (Sznol and Chen, 2013. Antagonist antibodies to PD-1 and B7-H1 (PD-L1) in the treatment of advanced human cancer. Clin Cancer Res; 19(5); 1021-34).

Programmed Cell Death 1 protein (PD-1) is a cell surface receptor that belongs to the CD28 family of receptors and is expressed on T cells and pro-B cells. PD-1 is presently known to bind two ligands, PD-L1 and PD-L2. PD-1, functioning as an immune checkpoint, plays an important role in down regulating the immune system by inhibiting the activation of T-cells, which in turn reduces autoimmunity and promotes self-tolerance. The inhibitory effect of PD-1 is thought to be accomplished through a dual mechanism of promoting apoptosis (programmed cell death) in antigen specific T-cells in lymph nodes while simultaneously reducing apoptosis in regulatory T cells (suppressor T cells). PD-1 is also known under a number of different aliases such as PDCD1; Programmed Cell Death 1; Systemic Lupus Erythematosus Susceptibility 2; Protein PD-1; HPD-1; PD1; Programmed Cell Death 1 Protein; CD279 Antigen; CD279; HPD-L; HSLE1; SLEB2; and PD-1. External Ids for PD-1 are HGNC: 8760; Entrez Gene: 5133; Ensembl: ENSG00000188389; OMIM: 600244; and UniProtKB: Q15116. New classes of drugs that block the activity of PD-1, the PD-1 inhibitors, activate the immune system to attack tumors and are therefore used with success to treat some types of cancer. Where reference herein is made to PD-1, the reference refers to human PD-1 unless otherwise stated. The PD-1 antigen binding site binds PD-1 and a variety of variants thereof, such as those expressed by some PD-1 positive tumor cells.

The binding of PD-L1 to PD-1 or B7.1 (CD80) transmits an inhibitory signal which reduces the proliferation of the PD-1 expressing T cells. PD-1 is thought to be able to control the accumulation of foreign antigen specific T cells through apoptosis. PD-L1 is expressed by a variety of cancer cells and the expression thereof is thought to be at least in part responsible for a dampening of an immune response against the cancer cell. PD-L1 is a member of the B7-family of protein and is known under a variety of other names such as CD274 Molecule; CD274 Antigen; B7 Homolog 1; PDCD1 Ligand 1; PDCD1LG1; PDCD1L1; B7H1; PDL1; Programmed Cell Death 1 Ligand 1; Programmed Death Ligand 1; B7-H1; and B7-H. External Ids for CD274 are HGNC: 17635; Entrez Gene: 29126; Ensembl: ENSG00000120217; OMIM: 605402; UniProtKB: Q9NZQ7. Where reference herein is made to PD-L1, the reference refers to human PD-L1 unless otherwise stated. The PD-L1 antigen binding site binds PD-L1 and a variety of variants thereof, such as those expressed by some PD-L1 positive tumor cells.

PD-L2 is a second ligand for PD-1. Engagement of PD-1 by PD-L2 inhibits T cell receptor (TCR)-mediated proliferation and cytokine production by CD4+ T cells. At low antigen concentrations, PD-L2/PD-1 binding inhibits B7-CD28 signals. At high antigen concentrations, PD-L2/PD-1 binding reduces cytokine production. PD-L expression is up-regulated on antigen-presenting cells by interferon gamma treatment. It is expressed in some normal tissues and a variety of tumors. PD-L1 and PD-L2 are thought to have overlapping functions and regulate T cell responses. The protein is known under a number of other names such as Programmed Cell Death 1 Ligand 2; B7 Dendritic Cell Molecule; Programmed Death Ligand 2; Butyrophilin B7-DC; PDCD1 Ligand 2; PD-1 Ligand 2; PDCD1L2; B7-DC; CD273; B7DC; PDL2; PD-1-Ligand 2; CD273 Antigen; BA574F11.2; and Btdc. External Ids for PD-L2 are HGNC: 18731; Entrez Gene: 80380; Ensembl: ENSG00000197646; OMIM: 605723; and UniProtKB: Q9BQ51. Where reference herein is made to PD-L2, the reference refers to human PD-L2 unless otherwise stated. The PD-L2 antigen binding site binds PD-L2 and a variety of variants thereof, such as those expressed by some PD-L2 positive tumor cells

### Immune Checkpoint Inhibitors

In certain aspects, said immune checkpoint inhibitor comprises a PD-L1, PD-L2 or PD-1 inhibitor. In certain aspects, the immune checkpoint inhibitor comprises or is an antibody. In certain aspects, the immune checkpoint inhibitor comprises or is an antibody that targets PD-L1, PD-L2 or PD-1. In certain aspects, the immune checkpoint inhibitor comprises or is an antibody that inhibits PD-L1, PD-L2 or PD-1.

In certain aspects, the PD-1, PD-L2 or PD-L1 inhibitor is an antibody or other binding molecule. In certain aspects, the immune checkpoint inhibitor comprises or is a PD-L1 inhibitor. In certain aspects, the immune checkpoint inhibitor comprises or is a PD-1 inhibitor. In certain aspects, the immune checkpoint inhibitor comprises or is an anti-PD-L1 antibody. In certain aspects, the immune checkpoint inhibitor comprises or is an anti-PD-1 antibody. In certain aspects, the immune checkpoint inhibitor is an antibody that binds or can bind PD-L1 or PD-1.

In certain aspects, the immune checkpoint inhibitor comprises or is nivolumab, pembrolizumab, cemiplimab, penpulimab, retifanlimab, sintilimab, tislelizumab, toripalimab, dostarlimab, atezolizumab, avelumab or durvalumab.

Immune checkpoint inhibitors, such as PD-1 inhibitors, are known in the art. They include antibodies that bind and block PD-1. Pembrolizumab, for instance, is a humanized antibody used in cancer immunotherapy that treats melanoma, lung cancer, head and neck cancer, Hodgkin's lymphoma, stomach cancer, and cervical cancer. It is given by slow injection into a vein. Pembrolizumab is a therapeutic antibody that binds to and blocks PD-1 located on lymphocytes. This receptor is a so-called “immune checkpoint”, and thus is generally responsible for preventing the immune system from attacking the body's own tissues. Normally, the PD-1 receptor on activated T-cells binds to the PD-L1 or PD-L2 ligands present on normal cells in the body, deactivating any potential cell-mediated immune response against these cells. Many cancers make proteins such as PD-L1 that also bind to the PD-1 receptor, thus shutting down the ability of the body to kill the cancer. Pembrolizumab works by inhibiting lymphocytes PD-1 receptors, blocking the ligands that would deactivate it and prevent an immune response. This allows the immune system to target and destroy cancer cells, but also blocks a key mechanism preventing the immune system from attacking the body itself.

Pembrolizumab was approved for medical use in the United States in 2014. In 2017, the US Food and Drug Administration (FDA) approved it for any unresectable or metastatic solid tumor with certain genetic anomalies (mismatch repair deficiency or microsatellite instability).

In certain aspects, the immune checkpoint inhibitor is nivolumab, pembrolizumab, cemiplimab, penpulimab, retifanlimab, sintilimab, tislelizumab, toripalimab or dostarlimab. In certain aspects, the immune checkpoint inhibitor is pembrolizumab. In certain aspects, the immune checkpoint inhibitor is nivolumab. In certain aspects, the immune checkpoint inhibitor is a PD-L1 inhibitor. In certain aspects, the PD-L1 inhibitor is atezolizumab, avelumab or durvalumab. The amino acid sequences of all said immune checkpoint inhibitors are well known in the art.

Durvalumab (sold under the brand name (tradename Imfinzi™) is an FDA-approved immune checkpoint inhibitor for treating cancer, like bladder and lung cancer. It is a human immunoglobulin G1 kappa (IgG1κ) monoclonal antibody that binds PD-L1 and blocks its interaction with PD-1 (CD279). Durvalumab is an immune checkpoint inhibitor or also referred to sometimes as an immune checkpoint inhibitor drug.

Pembrolizumab (sold under the brand name Keytruda™), is a humanized antibody used in cancer immunotherapy to treat a variety of cancers, including melanoma, lung cancer and Hodgkin lymphoma and functions as an immune checkpoint inhibitor. It is an IgG4 isotype antibody and targets the programmed cell death protein 1 (PD-1) receptor of lymphocytes. Pembrolizumab was approved for medical use in the United States in 2014. In 2017, the US Food and Drug Administration (FDA) approved it for any unresectable or metastatic solid tumor with certain genetic anomalies. It is on the World Health Organization's List of Essential Medicines.

Nivolumab (sold under the brand name Opdivo™), is an immune checkpoint inhibitor used to treat a number of cancers, including melanoma, lung cancer, malignant pleural mesothelioma, renal cell carcinoma, Hodgkin lymphoma, head and neck cancer, urothelial carcinoma, colon cancer, esophageal squamous cell carcinoma, liver cancer, gastric cancer, and esophageal or gastroesophageal junction (GEJ). Nivolumab is a human IgG4 monoclonal antibody that blocks PD-1. Nivolumab was approved for medical use in the United States in 2014. It is on the World Health Organization's List of Essential Medicines. Nivolumab is the second FDA-approved systemic therapy for mesothelioma and is the first FDA-approved immunotherapy for the first-line treatment of gastric cancer.

Atezolizumab (sold under the brand name Tecentriq™), is a monoclonal antibody medication used to treat urothelial carcinoma, non-small cell lung cancer (NSCLC), triple-negative breast cancer (TNBC), small cell lung cancer (SCLC), and hepatocellular carcinoma (HCC). It is a humanized, monoclonal antibody of the IgG1 isotype and targets programmed cell death-ligand 1 (PD-L1). Atezolizumab is the first PD-L1 inhibitor approved by the U.S. Food and Drug Administration.

Retifanlimab is (previously known as MGA012) is a humanized anti-PD-1 monoclonal antibody being developed for use as monotherapy as well as in combination with other cancer therapeutics. Retifanlimab is undergoing clinical trials (NCT04472429 and NCT04205812) as a monotherapy for patients with microsatellite instability-high endometrial cancer, Merkel cell carcinoma and squamous cell carcinoma of the anal canal (SCAC); and in combination with platinum-based chemotherapy for patients with non-small cell lung cancer and SCAC. Retifanlimab has been granted orphan drug designation by the FDA for the treatment of anal cancer and may be administered intravenously every four weeks at 500 mg (500 mg q4w).

Cemiplimab (sold under the brand name Libtayo®) is a monoclonal antibody medication for the treatment of squamous cell skin cancer. Cemiplimab belongs to a class of drugs that binds to the programmed death receptor-1 (PD-1), blocking the PD-1/PD-L1 pathway. In September 2018, it was approved by the FDA for treating people with metastatic cutaneous squamous cell carcinoma (CSCC) or locally advanced CSCC who are not candidates for curative surgery or curative radiation. It was approved for medical use in the European Union in June 2019.

Dostarlimab (Jemperli) is a PD-1-blocking antibody indicated for the treatment of adult patients with mismatch repair deficient (dMMR) recurrent or advanced endometrial cancer, as determined by an FDA-approved test, that has progressed on or following prior treatment with a platinum-containing regimen. Dosage and administration include dose one through four of 500 mg every three weeks (q3w), followed by subsequent dosing beginning three weeks after dose four (dose five onwards) at 1,000 mg every six weeks. Dosage form and strength are an injection of 500 mg/10 mL (50 mg/mL) solution in a single-dose vial.

Avelumab (or Bavencio®) is a PD-L1 blocking antibody indicated for Merkel cell carcinoma, urothelial carcinoma and renal cell carcinoma. Its dosage and indications include a premedication for the first four infusions and subsequently as needed. Avelumab is used for Merkel cell carcinoma, urothelial carcinoma and herein, as 800 mg every two weeks (q2w); 800 mg every 2 weeks. Optionally, Avelumab is used at 800 mg every two weeks in combination with axitinib 5 mg orally twice daily Avelumab is administered as an intravenous infusion over 60 minutes. The dosage form and strength is an injection of 200 mg/10 mL (20 mg/mL) solution in a single-dose vial.

Penpulimab is a humanized anti-PD-1 monoclonal antibody developed by Akeso Biopharma, in collaboration with Chia Tai Tianqing for the treatment of various cancers, including Hodgkin's lymphoma, nasopharyngeal cancer, non-small cell lung cancer (NSCLC) and solid tumors. Penpulimab is an immunoglobulin G1 monoclonal antibody engineered to completely eliminate Fcγ receptor binding and Fc-mediated effector functions that can compromise anti-tumor activity. In August 2021, penpulimab received its first approval in China for the treatment of adult patients with relapsed or refractory classic Hodgkin's lymphoma who have undergone at least second-line chemotherapy. The recommended dosage of penpulimab is 200 mg administered intravenously once every two weeks (200 mg q2w) until disease progression or intolerable toxicity occurs.

Sintilimab (Tyvyt®) is an anti-PD-1, fully human IgG4 monoclonal antibody that binds to PD-1 on the surface of T-cells, blocks the PD-1/PD-Ligand 1 (PD-L1) pathway, and reactivates T-cells to kill cancer cells. Sintilimab is developed by Innovent Biologics and Eli Lilly and Company and has been approved to treat relapsed or refractory classical Hodgkin lymphoma in patients who have undergone two or more lines of systemic chemotherapy by the National Medical Products Administration of China. Its instructions for use include a 200 mg, intravenous injection once every three weeks (q3w).

Tislelizumab is a humanized IgG4 anti-PD-1 monoclonal antibody developed both as a monotherapy and in combination with other therapies. The BLA submission was based on data from the Phase III RATIONALE 302 trial, which demonstrated a 30% reduction in the risk of death (HR=0.70, 95% CI: 0.57-0.85, p=0.0001) and extended median overall survival by 2.3 months compared to chemotherapy in people with unresectable recurrent locally advanced or metastatic esophageal squamous cell carcinoma who had received prior systemic therapy. Pharmacokinetics, safety and antitumor activity obtained from both phase IA and IB determined the tislelizumab recommended dose at 200 mg intravenous injection every three weeks (200 mg q3w) and as such was the dose and schedule recommended to be taken into subsequent clinical trials.

In certain aspects, the immune checkpoint inhibitor is a full length antibody or a fragment of an antibody, for example a Fab fragment or a single-chain variable fragment (scFv). In certain aspects, the immune checkpoint inhibitor according to the use or method of the present disclosure is a full length antibody. In certain aspects, the immune checkpoint inhibitor is, or comprises, an antibody selected from Nivolumab, Pembrolizumab, Cemiplimab, Dostarlimab, Atezolizumab, Avelumab, and Durvalumab or a functional fragment or variant thereof. In certain aspects, the immune checkpoint inhibitor is, or comprises, Pembrolizumab or a functional fragment or variant thereof.

In certain aspects, the immune checkpoint inhibitor comprises or consists of an amino acid sequence that is or has substantial sequence identity to the amino acid sequence of the antigen binding site of one of Nivolumab, Pembrolizumab, Cemiplimab, Dostarlimab, Atezolizumab, Avelumab, and Durvalumab. In certain aspects, the immune checkpoint inhibitor comprises or consists of an amino acid sequence that is or has substantial sequence identity to the amino acid sequence of the antigen binding site of Pembrolizumab.

Toripalimab (or Tuoyi™) is a selective, recombinant, humanized monoclonal antibody against PD-1 developed by Shanghai Junshi Bioscience Co., Ltd. Toripalimab is able to bind to PD-1 and block the interaction with its ligands. Toripalimab received a conditional approval in China for the treatment of melanoma in December, 2018. It has also received approvals to treat nasopharyngeal carcinoma and urothelial carcinoma in 2021. Additionally, several orphan drug designations were granted to toripalimab by the US Food and Drug Administration. The recommended phase II dose was determined to be 3 mg/kg Q2W in the first-in-human phase I trial NCT02836795. In said study, patients in the dose escalation cohorts received intravenous infusions at 1 mg/kg, 3 mg/kg, and 10 mg/kg Q2W; 28 days after the first dose, subjects continued to receive toripalimab at the intended dose level Q2W.

In certain aspects, the immune checkpoint inhibitor is, or comprises, at least one, more preferably at least two, even more preferably three heavy chain CDRs that are identical or substantially identical to the CDRs of one of Nivolumab, Pembrolizumab, Cemiplimab, Dostarlimab, Atezolizumab, Avelumab, and Durvalumab, in particular Pembrolizumab. In certain embodiments, the PD-1 or PD-L1 inhibitor comprises, at least one, more preferably at least two, even more preferably said three CDRs that are identical to the CDRs found in Pembrolizumab. In certain aspects, the immune checkpoint inhibitor is, or comprises, a heavy chain CDR1 that is identical to a CDR1 from one of Nivolumab, Pembrolizumab, Cemiplimab, Dostarlimab, Atezolizumab, Avelumab, and Durvalumab, a heavy chain CDR2 that is identical to a CDR2 from one of Nivolumab, Pembrolizumab, Cemiplimab, Dostarlimab, Atezolizumab, Avelumab, and Durvalumab, or a heavy chain CDR3 that is identical to a CDR3 from one of Nivolumab, Pembrolizumab, Cemiplimab, Dostarlimab, Atezolizumab, Avelumab, and Durvalumab. In certain aspects, the immune checkpoint inhibitor is, or comprises, a heavy chain CDR1 and a heavy chain CDR2 that are identical to a CDR1 and a CDR2 from one of Nivolumab, Pembrolizumab, Cemiplimab, Dostarlimab, Atezolizumab, Avelumab, and Durvalumab, a heavy chain CDR1 and a heavy chain CDR3 that are identical to a CDR1 and a CDR3 from one of Nivolumab, Pembrolizumab, Cemiplimab, Dostarlimab, Atezolizumab, Avelumab, and Durvalumab, or a heavy chain CDR2 and a heavy chain CDR3 that are identical to a CDR2 and a CDR3 from one of Nivolumab, Pembrolizumab, Cemiplimab, Dostarlimab, Atezolizumab, Avelumab, and Durvalumab. In certain aspects, the immune checkpoint inhibitor is, or comprises, a heavy chain CDR1, a heavy chain CDR2 and a heavy chain CDR3 that are identical to a CDR1, a CDR2 and a CDR3 from one of Nivolumab, Pembrolizumab, Cemiplimab, Dostarlimab, Atezolizumab, Avelumab, and Durvalumab. In certain aspects, the immune checkpoint inhibitor comprises heavy chain CDR1, CDR2 and CDR3 that are identical to CDR1, CDR2 and CDR3 from Pembrolizumab.

Methods for determining or annotating CDR sequences of the indicated antibodies are known by the skilled person using for instance an annotation system like IMGT, Chothia, Kabat or other suitable annotation systems.

In certain aspects, the immune checkpoint inhibitor is, or comprises, at least one, more preferably at least two, even more preferably three light chain CDRs that are identical or substantially identical to the CDRs of one of Nivolumab, Pembrolizumab, Cemiplimab, Dostarlimab, Atezolizumab, Avelumab, and Durvalumab, in particular Pembrolizumab. In certain embodiments, the PD-1 or PD-L1 inhibitor comprises, at least one, more preferably at least two, even more preferably said three CDRs that are identical to the CDRs found in Pembrolizumab. In certain aspects, the immune checkpoint inhibitor is, or comprises, a light chain CDR1 that is identical to a CDR1 from one of Nivolumab, Pembrolizumab, Cemiplimab, Dostarlimab, Atezolizumab, Avelumab, and Durvalumab, a light chain CDR2 that is identical to a CDR2 from one of Nivolumab, Pembrolizumab, Cemiplimab, Dostarlimab, Atezolizumab, Avelumab, and Durvalumab, or a light chain CDR3 that is identical to a CDR3 from one of Nivolumab, Pembrolizumab, Cemiplimab, Dostarlimab, Atezolizumab, Avelumab, and Durvalumab. In certain aspects, the immune checkpoint inhibitor is, or comprises, a light chain CDR1 and a light chain CDR2 that are identical to a CDR1 and a CDR2 from one of Nivolumab, Pembrolizumab, Cemiplimab, Dostarlimab, Atezolizumab, Avelumab, and Durvalumab, a light chain CDR1 and a light chain CDR3 that are identical to a CDR1 and a CDR3 from one of Nivolumab, Pembrolizumab, Cemiplimab, Dostarlimab, Atezolizumab, Avelumab, and Durvalumab, or a CDR2 and a CDR3 that are identical to a CDR2 and a CDR3 from one of Nivolumab, Pembrolizumab, Cemiplimab, Dostarlimab, Atezolizumab, Avelumab, and Durvalumab. In certain aspects, the immune checkpoint inhibitor is, or comprises, a light chain CDR1, a light chain CDR2 and a light chain CDR3 that are identical to a CDR1, a CDR2 and a CDR3 from one of Nivolumab, Pembrolizumab, Cemiplimab, Dostarlimab, Atezolizumab, Avelumab, and Durvalumab. In certain aspects, the immune checkpoint inhibitor comprises light chain CDR1, light chain CDR2 and light chain CDR3 that are identical to CDR1, CDR2 and CDR3 from Pembrolizumab.

In certain aspects, the immune checkpoint inhibitor is administered pursuant to the present recommendation set by FDA approval. Said recommendations are typically comprised by instructions for use of in the treatment of cancer in a subject. Therefore, in certain aspects, the present disclosure relates to instructions for use of said immune checkpoint inhibitor.

### Cancers of the Present Disclosure

The words cancer and tumor are used herein and typically both refer to cancer, unless otherwise specifically stated.

Cancers that are known collectively as head and neck cancers usually originate in the squamous cells that line the moist, mucosal surfaces inside the head and neck, such as inside the mouth, the nose, and the throat. These squamous cell cancers are often referred to as squamous cell carcinomas of the head and neck and said cancers are treated in certain aspects of the present disclosure. Although rare, head and neck cancers can also occur in the salivary glands. In certain aspects, the head and neck cancer may occur in the oral cavity. This includes the lips, the front two-thirds of the tongue, the gums, the lining inside the cheeks and lips, the floor of the mouth under the tongue, the hard palate, and the small area of the gum behind the wisdom teeth.

Thus, in certain aspects, the head and neck cancer is squamous cell carcinoma and includes laryngeal cancer, hypopharyngeal cancer, nasal cavity cancer, paranasal sinus cancer, oral cancer, oropharyngeal cancer or salivary gland cancer. In certain aspects, the present disclosure relates to treatment of a cancer comprising a squamous cell head and neck cancer, such as located in the oropharynx, hypopharynx, the larynx, the oral cavity or the tongue.

In certain aspects, the cancer comprises or is an adenocarcinoma, a squamous cell carcinoma, or a head and neck cancer, including squamous cell carcinoma of the head and neck (SCCHN).

In certain aspects, the cancer is a squamous cell carcinoma of the head and neck. In certain aspects, the cancer is a squamous cell carcinoma of the head and neck that expresses EGFR and PD-L1. In certain aspects, the cancer is a squamous cell carcinoma that expresses EGFR and PD-L1.

In certain aspects, the cancer is a cancer of the pharynx, including oropharynx and hypopharynx, oral cavity, larynx, paranasal sinuses, nasal cavity or salivary glands.

In certain aspects, the cancer is a cancer of the oropharynx, oral cavity, hypopharynx or larynx.

In certain aspects, the primary location of the cancer is in the oropharynx, oral cavity, hypopharynx or larynx. In certain aspects, the head and neck cancer is a squamous cell carcinoma of unknown primary (also referred to in the art as a cancer of unknown primary or CUP).

In certain aspects, the cancer is locally advanced, unresectable or metastatic cancer, such as head and neck squamous cell carcinoma.

### Expression of Targets of the Present Disclosure by Cancers

In certain aspects, the cancer expresses EGFR, LGR5 and/or PD-L1. In certain aspects, the cancer expresses EGFR. In certain aspects, the cancer expresses LGR5.

In certain aspects, the cancer expresses PD-L1. In certain aspects, the cancer expresses EGFR and PD-L1. In certain aspects, the cancer expresses EGFR, LGR5 and PD-L1.

As used herein, a cancer expresses EGFR if the cancer comprises cells that express EGFR. A cell which expresses EGFR comprises detectable levels of RNA that codes for EGFR. In certain aspects, EGFR expression is determined by ISH. As used herein, a cancer expresses LGR5 if the cancer comprises cells that express LGR5. A cell which expresses LGR5 comprises detectable levels of RNA that codes for LGR5.

In certain aspects, EGFR protein expression is detected by IHC. In certain aspects, EGFR expression is determined by IHC using a commercially available EGFR detection kit, such as the EGFR pharmDx™ kit for a Dako autostainer (Agilent), using the manufacturer's recommendations or the commercially available IHC EGFR detection kit based on EGFR clone 113 which binds the EGFR extracellular domain (Leica, https://shop.leicabiosystems.com/us/ihc-ish/ihc-primary-antibodies/pid-epidermal-growth-factor-receptor). Alternatively, EGFR expression is determined using the Novocastra™ Liquid Mouse Monoclonal Antibody Epidermal Growth Factor Receptor which is based on clone EGFR.113 (Product Code: NCL-L-EGFR, Epidermal Growth Factor Receptor—IHC Primary Antibodies by leicabiosystems.com).

Briefly, the commercially available EGFR pharmDx™ IHC kit system contains reagents required to complete an IHC staining procedure for routinely-fixed, paraffin-embedded specimens. Following incubation with the primary, non-Her2, Her3 and Her4 cross-reactive, monoclonal antibody (clone 2-18C9) to human EGFR protein, this kit employs a ready-to-use visualization reagent based on dextran technology. This reagent consists of both secondary goat anti-mouse antibody molecules and horseradish peroxidase molecules linked to a common dextran polymer backbone. The enzymatic conversion of the subsequently added chromogen results in formation of a visible reaction product at the antigen site. Results are routinely assessed using a light microscope. Control slides containing two formalin-fixed, paraffin-embedded human cell lines with staining intensity scores of 2+ and 0 are provided for quality control of the kit reagent performance.

Staining intensity is established as follows: 3+(strong staining): visible at low levels of magnification, ×5 objective lens which could be confirmed at higher levels as required; 2+(moderate staining): visible at intermediate levels of magnification, ×10 or ×20 objective lenses; 1+(weak staining): only reliably confirmable at high magnification, ×40 objective lens; 0 (no staining): no staining visible at high magnification.

In certain aspects, EGFR expression is determined using immunohistochemistry (IHC) and the cancer is IHC positive for EGFR. In certain aspects, the cancer is characterized by an EGFR IHC score of 2+ or 3+. In certain aspects, the cancer is characterized by an H score for EGFR of more than 50, more than 80 or more than 200, but not more than 300.

In certain aspects, EGFR expression is determined using immunohistochemistry (IHC) followed by assigning an H-score for EGFR using a range of 0-300. In certain aspects, the cancer of the present disclosure is a cancer characterized by an H-score for EGFR of more than 200 on a scale of 0-300. In certain aspects, the EGFR H score is thus >200 up to and including 300. In certain aspects, the cancer of the present disclosure is characterized by an H-score for EGFR of more than 50 on a scale of 0-300. In certain aspects, the cancer of the present disclosure is head and neck cancer characterized by an H-score for EGFR of more than 50 on a scale of 0-300. In certain aspects, the cancer of the present disclosure is characterized by an H-score for EGFR of more than 80 on a scale of 0-300. In certain aspects, the cancer of the present disclosure is head and neck cancer characterized by an H-score for EGFR of more than 80 on a scale of 0-300.

In another aspect, the cancer is a head and neck cancer characterized by an EGFR IHC score of 2+ or 3+.

Herein, determining the H-score to assign the EGFR expression status involves a first step of establishing intensity of membrane staining (resulting in a scoring of 0, 1+, 2+, or 3+) which is determined for each cell in a predefined field as described herein. Subsequently, the percentage of cells at each staining intensity level is calculated, and finally, an H-score is assigned using the following formula: [1×(% cells having 1+ staining)+2×(% cells having 2+ staining)+3×(% cells having 3+ staining)] resulting in an H-score for EGFR between 0-300. As a result, the H-score gives more relative weight to higher intensity or amount of staining in a given tumor sample.

Optionally, the treatment with the antibody or functional part, derivative and/or analogue thereof comprises (or in certain aspects is preceded by) a step of diagnosing the subject for EGFR status. In certain aspects, subjects having an IHC score of 3+, or said cancer is characterized by an H-score for EGFR of more than 200, on a scale of 0-300, are selected for treatment. In certain aspects, the treatment of a subject is preceded by a step of diagnosing said subject of having an H-score for EGFR of more than 200 on a scale of 0-300.

In certain aspects, the cancer expresses LGR5. As used herein, a cancer expresses LGR5 if the cancer comprises cells that express LGR5. A cell which expresses LGR5 comprises detectable levels of RNA that codes for LGR5.

Expression can often also be detected by incubating the cell with an antibody that binds to LGR5. However, some cells do not express the protein high enough for such an antibody test. In such cases mRNA or other forms of nucleic acid sequence detection is preferred. In certain aspects, LGR5 is detected via mRNA expression. In certain aspects, LGR5 detection is by RNA sequencing. In certain aspects, LGR5 detection is by Tissue MicroArray (TMA) staining. In certain aspects, LGR5 expression is determined using In-Situ Hybridization (ISH). Thus preferably, the cancer is ISH positive for LGR5. ISH positive preferably means that expression is characterized by an H-score of 1 or more.

Techniques for detection and scoring on the basis of TMA, ISH and IHC are each well known in the art to the person of ordinary skill and typically commercially available as a standard kit. Quantifying mRNA levels using ISH and expressing such on the basis of an H-score, such as for LGR5, can be performed using commercially available kits, such as the RNAscope® kit from Advanced Cell Diagnostics (Hayward, CA, USA) on a staining platform such as the BondRx platform (Leica, Wetzlar, Germany). Typically, the ISH H-score for LGR5 quantification ranges from 0-400. Alternatively, LGR5 expression is determined by RNA sequencing (RNAseq).

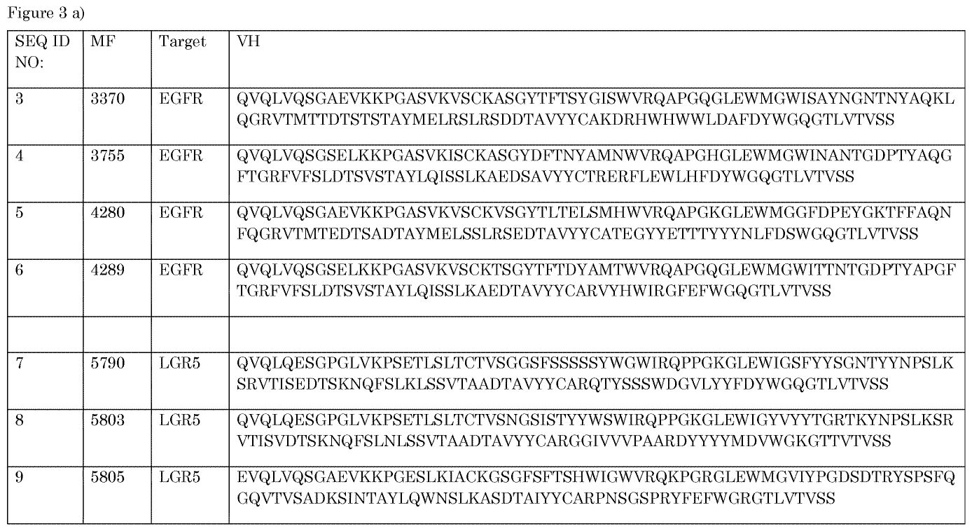

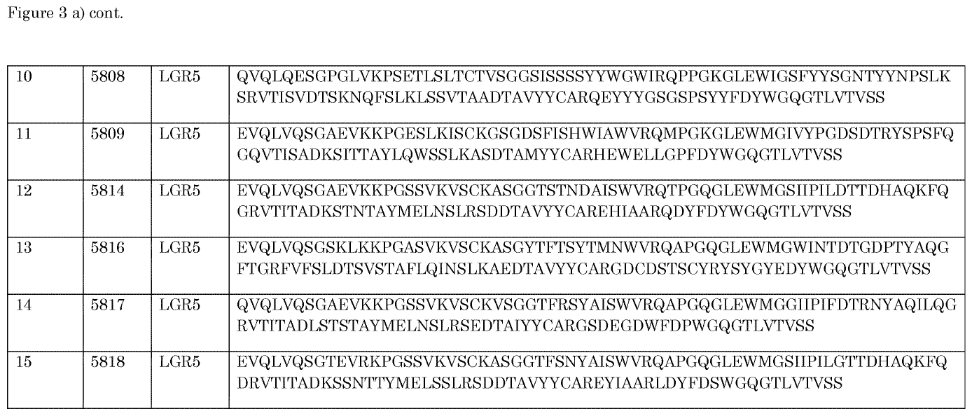

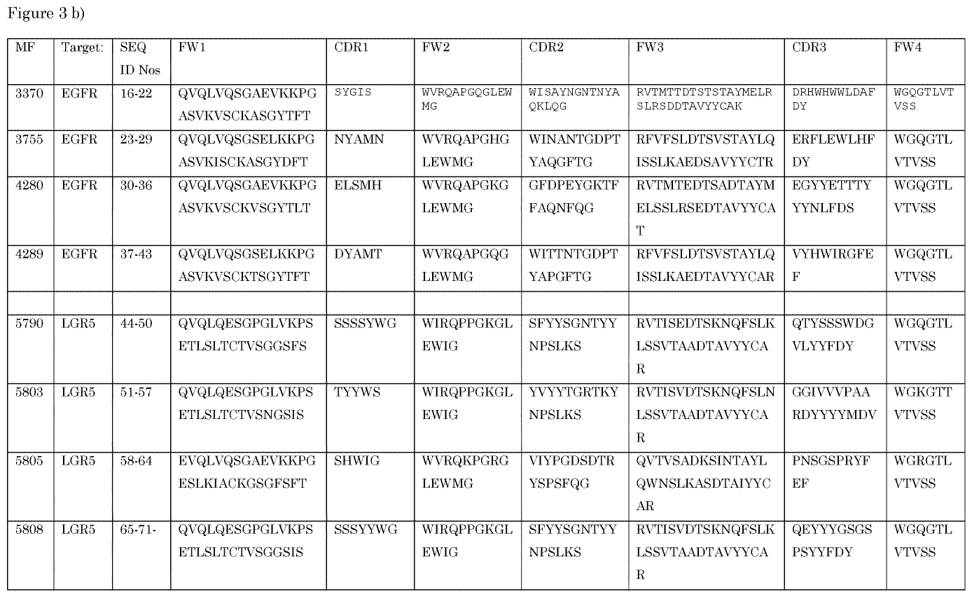

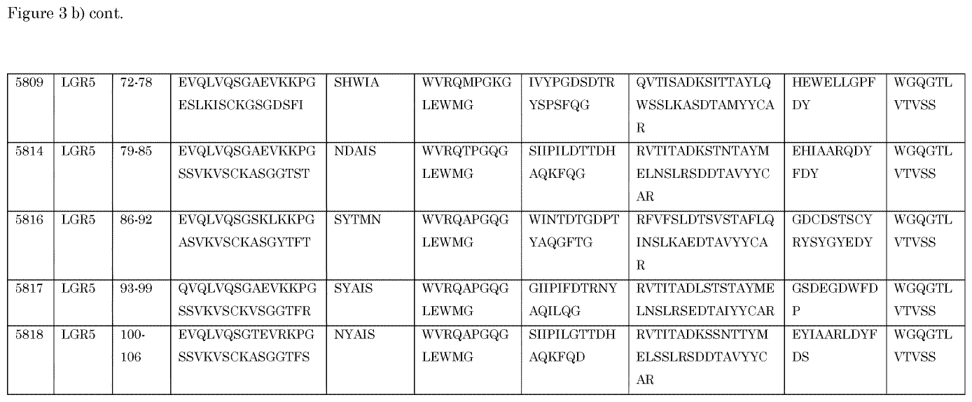

In certain aspects, the cancer expresses LGR5 in sufficient levels for an antibody to bind the LGR5 protein, such as an antibody comprising a VH chain of the variable domain that binds LGR5 that comprises the amino acid sequence of the VH chain of MF5816 as depicted in FIG. 3, or alternative variable domains that bind LGR5 set out herein. In certain aspects, the cancer expresses EGFR in sufficient levels for an antibody to bind the EGFR protein, such as an antibody comprising a VH chain of the variable domain that binds EGFR that comprises the amino acid sequence of the VH chain of MF3755 as depicted in FIG. 3, or alternative variable domains that bind EGFR set out herein.

In certain aspects, the cancer expresses PD-L1. In certain aspects, PD-L1 protein expression is detected by IHC. In certain aspects, Combined Positive Score (CPS) scoring is performed on a sample obtained from said subject. In certain aspects, the CPS is determined by an FDA approved test. In certain aspects, the CPS is determined using IHC, in particular using clone 22C3 (Agilent). In certain aspects, the CPS is determined using pharmDx PD-L1 IHC kit using clone 22C3. In certain aspects, the Combined Positive Score is determined according to Example 4. In certain aspects, PD-L1 expression is determined by IHC using a commercially available PD-L1 detection kit, such as the PD-L1 IHC 22C3 pharmDx™ assay (Agilent Technologies, Carpinteria, CA, USA) for a Dako autostainer using the manufacturer's recommendations.

In certain aspects, the cancer has a CPS for PD-L1 expression of 1 or more, but not more than 100. CPS herein is the number of PD-L1 staining cells (tumor cells, lymphocytes, macrophages) divided by the total number of viable tumor cells, multiplied by 100. Although the result of the calculation can exceed 100, the maximum score is defined as CPS 100. In certain aspects, CPS is determined using IHC. In certain aspects, CPS is determined by IHC using clone 22C3. In certain aspects, said cancer has a CPS score of between ≥1 and <20. In certain aspects, said cancer has a CPS score of between ≥20 and 100. In certain aspects, said subject is positive for p16 status as determined on a sample obtained from the subject or cancer in particular in case of oropharyngeal cancer, as is described herein below. In certain aspects, said subject is negative for p16 status as determined on a sample obtained from the subject or cancer in particular in case of a head and neck cancer other than oropharyngeal cancer.

In certain aspects, CPS is determined by IHC using clone 22C3. In certain aspects, PD-L1 protein expression is detected by IHC using PD-L1 IHC 22C3 which is a qualitative immunohistochemical assay using monoclonal mouse anti-PD-L1, clone 22C3 intended for use in the detection of PD-L1 protein in formalin-fixed, paraffin-embedded (FFPE) cancer tissues using EnVision FLEX visualization system on Autostainer Link 48.

In short, the PD-L1 IHC 22C3 pharmDx kit includes reagents required for the immunohistochemical staining (except wash buffer), control slides representing different expression levels of PD-L1 protein, and detailed instructions. The kit has been tailored especially for use on Autostainer Link 48 instruments. PD-L1 IHC 22C3 pharmDx contains optimized reagents and protocol required to complete an IHC staining procedure of FFPE specimens using Autostainer Link 48 and PT link pre-treatment module. Following incubation with the primary monoclonal antibody to PD-L1 or the negative control reagent, specimens are incubated with a linker antibody specific to the host species of the primary antibody, and then are incubated with a ready-to-use visualization reagent consisting of secondary antibody molecules and horseradish peroxidase molecules coupled to a dextran polymer backbone. The enzymatic conversion of the subsequently added chromogen results in precipitation of a visible reaction product at the site of the antigen. The color of the chromogenic reaction is modified by a chromogen enhancement reagent. The specimen may then be counterstained and coverslipped. Results are interpreted using a light microscope. Control Slides containing two FFPE human cell lines are provided to validate staining runs.

In certain aspects, CPS is determined by IHC using clone 28-8. In certain aspects, PD-L1 protein expression is detected by IHC using PD-L1 IHC 28-8 which is a qualitative immunohistochemical assay using monoclonal rabbit anti-PD-L1, clone 28-8 intended for use in the detection of PD-L1 protein in formalin-fixed, paraffin-embedded (FFPE) squamous cell carcinoma of the head and neck (SCCHN) tissues using EnVision FLEX visualization system on Autostainer Link 48.

In certain aspects, a sample obtained from a subject having a cancer of the present disclosure is tested for their PD-L1 CPS score and tested for p16 status. Relevant CPS scoring herein is between 21 and 100, or between ≥1 and <20 or a CPS score of between ≥20 and 100. Relevant p16 status outcomes are p16 positive or negative.

Thus, in certain aspects, a subject having a cancer which is to be treated following a method or use of the present disclosure, has a CPS score of between ≥1 and 100, and is positive for p16 status.

Alternatively, in certain aspects, a subject having a cancer which is to be treated following a method or use of the present disclosure, has a CPS score of between ≥1 and 100, and is negative for p16 status.

Alternatively, in certain aspects, a subject having a cancer which is to be treated following a method or use of the present disclosure, has a CPS score of between ≥1 and <20, and is positive for p16 status.

Alternatively, in certain aspects, a subject having a cancer which is to be treated following a method or use of the present disclosure, has a CPS score of ≥1 and <20 and is negative for p16 status.

Alternatively, in certain aspects, a subject having a cancer which is to be treated following a method or use of the present disclosure, has a CPS score of between ≥20 and 100, and is positive for p16 status.

Alternatively, in certain aspects, a subject having a cancer which is to be treated following a method or use of the present disclosure, has a CPS score of between ≥20 and 100, and is negative for p16 status.

Optionally, the treatment with the antibody or functional part, derivative and/or analogue thereof and said immune checkpoint inhibitor comprises a step of diagnosing the subject for PD-L1 status. In certain aspects, the treatment is preceded by a step of said diagnosing for PD-L1 status. In certain aspects, subjects having head and neck cancer with a CPS score of at least 1, and not more than 100, are selected for treatment. In certain aspects, the treatment of a subject is preceded by a step of diagnosing said subject for having a CPS score for PD-L1 of between 1 and 100. In certain aspects, said subject is diagnosed for PD-L1 status by having a CPS score of between ≥1 and <20 or a CPS score of between ≥20 and 100. Furthermore, treatment with the antibody or functional part, derivative and/or analogue thereof and said immune checkpoint inhibitor is optionally preceded by a step of diagnosing the subject for p16 status. Said subject may be determined or diagnosed to be p16 positive or negative.

In certain aspects, the present disclosure provides a method of diagnosing a subject having a head and neck cancer and selecting said subject for treatment with an antibody or functional part, derivative and/or analogue thereof as disclosed herein and an immune checkpoint inhibitor as disclosed herein, comprising the step of determining the CPS score for PD-L1 (of a sample obtained from the subject or cancer), selecting said subject for said treatment if said CPS score is 1 or more, optionally not more than 100, and treating said subject for said cancer. In certain aspects, said subject is selected for treatment in case a CPS score of between 21 and <20 is established. In certain aspects, said subject is selected for treatment in case a CPS score of between ≥20 and 100 is established. Furthermore, diagnosing and selecting a subject optionally comprises a step of determining p16 status of a sample obtained from the subject or cancer and selecting said subject for treatment if said p16 status is positive, in particular in case of oropharyngeal cancer. Furthermore, diagnosing and selecting a subject optionally comprises a step of determining p16 status of a sample obtained from the subject or cancer and selecting said subject for treatment if said p16 status is negative, in particular in case of a head and neck cancer other than oropharyngeal cancer.

Also provided is a method of selecting a subject having a head and neck cancer for treatment with an antibody or functional part, derivative and/or analogue thereof that comprises a variable domain that binds an extracellular part of EGFR as disclosed herein and an immune checkpoint inhibitor as disclosed herein, the method comprising: a) determining the Combined Positive Score (CPS) for PD-L1 expression in a sample obtained from the subject; and b) selecting the subject for said treatment if the sample has a CPS for PD-L1 expression of 1 or more, but optionally not more than 100. In certain aspects, said subject has a CPS score of between ≥1 and <20 and is selected for treatment or has a CPS score of between ≥20 and 100 is selected for treatment. Furthermore, said method may optionally comprise a further step of determining p16 status of a sample obtained from the subject or cancer and selecting said subject for treatment if said p16 status is positive, in particular in case of oropharyngeal cancer. Furthermore, said method may optionally comprises a further step of determining p16 status of a sample obtained from the subject or cancer and selecting said subject for treatment if said p16 status is negative, in particular in case of a head and neck cancer other than oropharyngeal cancer.

Also provided is a method of establishing whether a subject having a head and neck cancer is likely to respond to treatment with a head and neck cancer for treatment with an antibody or functional part, derivative and/or analogue thereof as disclosed herein that comprises a variable domain that binds an extracellular part of EGFR and an immune checkpoint inhibitor as disclosed herein, the method comprising: a) determining the Combined Positive Score (CPS) for PD-L1 expression in a sample obtained from the subject; and b) selecting a sample exhibiting CPS expression of PD-L1 of 1 or more, but optionally not more than 100, thereby establishing that the subject from which the sample is derived is likely to respond to said treatment. In certain aspects, said subject has a CPS score of between ≥1 and <20, thereby establishing that said subject is to likely respond to said treatment. In certain aspects, said subject has a CPS score of between ≥20 and 100, thereby establishing that said subject is to likely respond to said treatment. Furthermore, said method may optionally comprises a further step of determining p16 status of a sample obtained from the subject or cancer. In certain aspects, said subject has a p16 positive status, in particular in case of oropharyngeal cancer, thereby establishing that said subject is to likely respond to said treatment. In certain aspects, said subject has a p16 negative status, in particular in case of a head and neck cancer other than oropharyngeal cancer, thereby establishing that said subject is to likely respond to said treatment.

Also provided is a method of classifying subjects having a head and neck cancer on the basis of the Combined Positive Score for PD-L1 expression prior to treatment with an antibody or functional part, derivative and/or analogue thereof as disclosed herein that comprises a variable domain that binds an extracellular part of EGFR and an immune checkpoint inhibitor as disclosed herein, the method comprising: a) determining the Combined Positive Score for PD-L1 expression in a sample obtained from the subject; and b) classifying the subject from which the sample was obtained as eligible for said treatment if the sample exhibits CPS expression for PD-L1 of 1 or more, but optionally not more than 100. In certain aspects, in step b) said subject is classified as eligible for said treatment if the sample exhibits CPS expression for PD-L1 of between ≥1 and <20. In certain aspects, in step b) said subject is classified as eligible for said treatment if the sample exhibits CPS expression for PD-L1 of between ≥20 and 100. Furthermore, said method may optionally comprises a further step of determining p16 status of a sample obtained from the subject or cancer. In certain aspects, said subject has a p16 positive status, in particular in case of oropharyngeal cancer, thereby classifying said subject as eligible for said treatment. In certain aspects, said subject has a p16 negative status, in particular in case of a head and neck cancer other than oropharyngeal cancer, thereby classifying said subject as eligible for said treatment.

In certain aspects, the Combined Positive Score is determined by an FDA approved test. In certain aspects, the Combined Positive Score is determined using IHC, in particular using clone 22C3.

In certain aspects, CPS is histologically confirmed CPS having a value of 1 or more, optionally not more than 100, wherein CPS is defined as the number of PD-L1 positive tumor cells, lymphocytes, and macrophages divided by the total number of tumor cells, multiplied by 100.

In certain aspects, subjects of the present disclosure have a PD-L1 CPS score of ≥1 and <20. In certain aspects, subjects of the present disclosure have a PD-L1 CPS score of CPS≥20 but not more than for instance a CPS score of 100.

p16 (INK4a, Cyclin-dependent kinase inhibitor 2A (CDKN2A)) is typically used as a marker for HPV status. In certain aspects, p16 status is determined by immunohistochemistry (IHC). In certain aspects, testing for p16 IHC status is based on p16/HPV testing for Head & Neck Carcinomas following CAP Guideline 2018. IHC testing for p16 status may be performed using any commercially available test according to the manufacturer's instructions. Examples include commercially available test kits, such as an IHC test based on clone E6H4 (CINtec® Histology, Roche Diagnostics) or anti-p16 primary antibody 6H12 (e.g. Catalog No: PA0016, Leica Biosystems).

In certain aspects, testing for p16 status follows CAP Guideline 2018 (Lewis et al., Human Papillomavirus Testing in Head and Neck Carcinomas; Guideline From the College of American Pathologists, Arch Pathol Lab Med, Vol 142, May 2018). In certain aspects, Statement 8 of said Guideline is followed which states that a pathologist should report p16 IHC positivity as a surrogate for high risk HPV in tissue specimens when there is at least 70% nuclear and cytoplasmic expression with at least moderate to strong intensity. In certain aspects, p16 testing is performed on a sample obtained from a subject suffering from oropharyngeal cancer or on a oropharyngeal cancer/tumor sample. In certain aspects, testing for p16 status is according to Example 6.

### Previous Treatments

In certain aspects, the subject has not received prior anti-cancer treatment for treatment of said cancer.

In certain aspects, the subject has not received previous treatment with an anti PD-L1, anti PD-1 or anti-EGFR therapy. The treatment of the present disclosure is particularly effective in a group of subjects that have not yet received any previous anti-cancer treatment. Also, exposure of a treatment-naïve cancer to the treatment of the present disclosure is particularly effective. A therapy that involves subjects or cancers that have not received previous anti-cancer treatment is also referred to as first-line treatment. Hence, in certain aspects, the antibody or functional part, derivative and/or analogue thereof and immune checkpoint inhibitor of the present disclosure, are used as first-line treatment.

In certain aspects, the subject has not received prior anti-cancer treatment for treatment of said cancer at least six months prior to receiving said antibody or functional part, derivative and/or analogue thereof and said immune checkpoint inhibitor.

In certain aspects, said previous anti-cancer treatment comprises chemotherapy, immune therapy, an anti-EGFR agent, an antibody targeting EGFR, cetuximab, a PD-1 inhibitor or a PD-L1 inhibitor.

In certain aspects, the subject has previously not been treated with an anti-EGFR agent. In certain aspects, the subject has not been treated with an antibody targeting EGFR. In certain aspects, the subject has not been treated with cetuximab. Such a subject is also referred to as a cetuximab-naïve or anti-EGFR-naïve subject. Worded differently, the cancer of said subject has previously not been treated with any anti-cancer agent, such as an anti-EGFR agent. In certain aspects, the cancer of said subject has not been treated with an antibody targeting EGFR. In certain aspects, the cancer of said subject has not been treated with cetuximab. Such a subject is also referred to as a cetuximab-naïve or anti-EGFR-naïve subject.

In certain aspects, the subject of the present disclosure has not received prior treatment with anticancer immune therapy. In certain aspects, said immune therapy comprises an anti-PD-L1 or anti-PD-1 treatment, including pembrolizumab, nivolumab, atezolizumab, retifanlimab, cemiplimab or other anti-PD1, anti-PD-L1 antibodies approved or in development. In certain aspects, the anticancer immune therapy comprises an immune checkpoint inhibitor, including prior pembrolizumab treatment.

In certain aspects, the subject of the present disclosure has not received prior anti-cancer treatment for treatment of said cancer at least six months prior to receiving said antibody or functional part, derivative and/or analogue thereof and said immune checkpoint inhibitor. In certain aspects, said subject has received multimodal treatment, which comprises surgery, radiotherapy and/or platinum-containing chemotherapy, such as cisplatin, more than six months prior to receiving said antibody or functional part, derivative and/or analogue thereof and said immune checkpoint inhibitor. In certain aspects, said subject underwent said multimodal treatment to treat curable local disease.

The Antibody or Functional Part, Derivative and/or Analogue Thereof of the Present Disclosure that Binds EGFR and Optionally LGR5.

In certain aspects, the antibody or functional part, derivative and/or analogue thereof, comprises a variable domain that binds an extracellular part of EGFR. Such variable domains are described further herein. In certain aspects, the antibody or functional part, derivative and/or analogue thereof, further comprises a variable domain that binds an extracellular part of LGR5. Such variable domains are described further herein. In certain aspects, the antibody or functional part, derivative and/or analogue thereof, comprises a variable domain that binds an extracellular part of EGFR and a variable domain that binds an extracellular part of LGR5.

In certain aspects, the antibody or functional part, derivative and/or analogue thereof, comprises or is a multispecific antibody. In certain aspects, the antibody or functional part, derivative and/or analogue thereof, comprises or is a bispecific antibody.

In certain aspects, the antibody or functional part, derivative and/or analogue thereof as disclosed herein is a multispecific antibody. In certain aspects, said antibody is a bispecific antibody. Said multi- or bispecific antibody or a functional part, derivative and/or analogue thereof, in certain aspects comprises a variable domain that binds an extracellular part of the epidermal growth factor (EGF) receptor and a variable domain, which in certain aspects, does not bind EGFR. In certain aspects, the antibody or functional part, derivative and/or analogue thereof binds EGFR monovalently. Also, in certain aspects, said multispecific or bispecific antibody or functional part, derivative and/or analogue thereof, comprises a variable domain that binds LGR5.

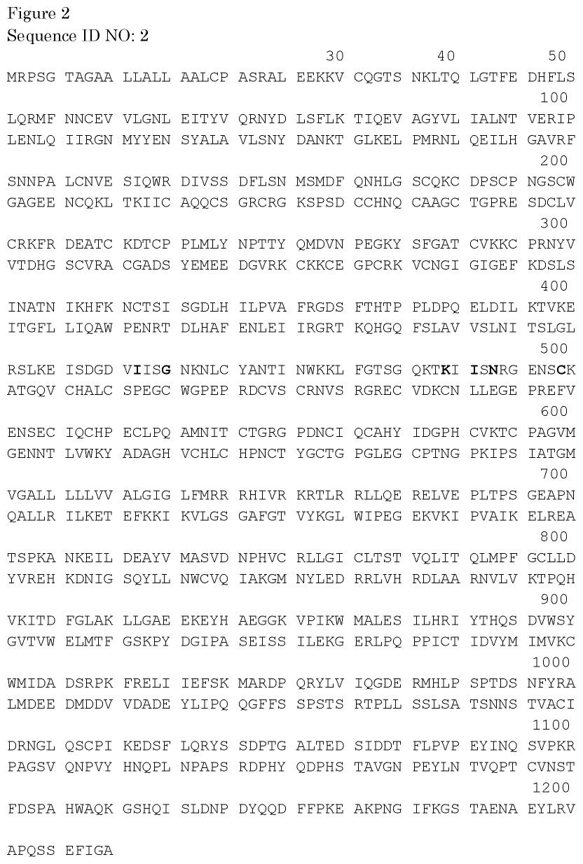

In certain aspects, the EGFR is a human EGFR. The EGFR that is bound by said antibody or functional part, derivative and/or analogue thereof of the present disclosure, includes wildtype EGFR as well as EGFR having an oncogenic driver mutation. In certain aspects, said oncogenic driver mutation is an activating EGFR mutation. In certain aspects, such a mutation does not conformationally change the epitope that is bound by the antibody of the present disclosure. In certain aspects, the EGFR mutations of the present disclosure include mutations such as exon 18 mutations, including G719A, G719C, 2E709_T710D, E709A, G719S; exon 19 deletion mutations, including deletion of LREA or VAIKEL; exon 19 point mutations G735S, P753L, L747S, D761Y; in-frame exon 20 insertion mutations of 1-7 amino acids, exon 20 point mutations, including V765A, T783A, V774A, S784P, V769M, T790M; exon 21 mutations, including L858R, T854A, A871E, L861A, L861C, L861S, V843I or P848L. The antibody of the present disclosure binds an epitope that is not located in close proximity of said mutations. In particular, the EGFR mutation is S492R, which results in loss of binding of cetuximab to EGFR. The antibody of the present disclosure binds an epitope that is different from the epitope that is recognized by Cetuximab. Without being bound by any theory, it is believed that amino acid residues 1462; G465; K489; 1491; N493; and C499 as depicted FIG. 2 are involved in binding an epitope by an antibody of the present disclosure. In certain aspects, involvement in binding is determined by observing a reduced binding of the variable domain to an EGFR with one or more of the amino acid residue substitutions selected from I462A; G465A; K489A; I491A; N493A; and C499A. In certain aspects, epitope binding for EGFR as well as LGR5 is established using shotgun mutagenesis analysis.

In one aspect, the variable domain that binds an epitope on an extracellular part of human EGFR is a variable domain that binds an epitope that is located within amino acid residues 420-480 of the sequence depicted in FIG. 2. In certain aspects, the binding of the variable domain to EGFR is reduced by one or more of the following amino acid residue substitutions I462A; G465A; K489A; I491A; N493A; and C499A in EGFR. In certain aspects, binding of the antibody to human EGFR interferes with the binding of EGF to the receptor. In certain aspects, the epitope on EGFR is a conformational epitope. In one aspect, the epitope is located within amino acid residues 420-480 of the sequence depicted in FIG. 2, or within 430-480 of the sequence depicted in FIG. 2. In certain aspects, said epitope is located within 438-469 of the sequence depicted in FIG. 2.

Without being bound by theory it is believed that the contact residues of the epitope, i.e. where the variable domain contacts the human EGFR are likely 1462; K489; 1491; and N493. The amino acid residues G465 and C499 are likely indirectly involved in the binding of the antibody to EGFR.

In certain aspects, the variable domain binds LGR5. In certain aspects, the LGR5 is a human LGR5. The multispecific or bispecific antibody or a functional part, derivative and/or analogue thereof as described herein comprises a variable domain that binds an extracellular part of a human epidermal growth factor (EGF) receptor and in certain aspects, a variable domain that binds a human LGR5.

In certain aspects, the antibody or a functional part, derivative and/or analogue thereof as described herein comprises a variable domain that binds an extracellular part of the epidermal growth factor (EGF) receptor and interferes with the binding of EGF to the receptor and a variable domain that binds LGR5 wherein interaction of the antibody with LGR5 on an LGR5-expressing cell does not block the binding of an Rspondin (RSPO) to LGR5. Methods for determining whether an antibody blocks or does not block the binding of an Rspondin to LGR5 are described in WO2017069528, which is hereby incorporated by reference.

Where herein accession numbers or alternative names of proteins/genes are given, they are primarily given to provide a further method of identification of the mentioned protein as a target, the actual sequence of the target protein bound by an antibody of the invention may vary, for instance because of a mutation and/or alternative splicing in the encoding gene such as those occurring in some cancers or the like. The target protein is bound by the antibody as long as the epitope is present in the protein and the epitope is accessible to the antibody.

In certain aspects, an antibody or a functional part, derivative and/or analogue thereof as described herein interferes with the binding of a ligand for EGFR to EGFR. The term “interferes with binding” as used herein means that binding of the antibody or a functional part, derivative and/or analogue thereof to the EGFR competes with the ligand for binding to EGF receptor. The antibody or a functional part, derivative and/or analogue thereof may diminish ligand binding, displace ligand when this is already bound to the EGF receptor or it may, for instance through steric hindrance, at least partially prevent that ligand can bind to the EGF receptor.

In certain aspects, an EGFR antibody as disclosed herein inhibits respectively EGFR ligand-induced signaling, measured as ligand-induced growth of BxPC3 cells (ATCC CRL-1687) or BxPC3-luc2 cells (Perkin Elmer 125058) or ligand-induced cell death of A431 cells (ATCC CRL-1555). EGFR can bind a number of ligands and stimulate growth of the mentioned BxPC3 cells or BxPC3-luc2 cells. In the presence of an EGFR ligand the growth of BxPC3 or BxPC3-luc2 cells is stimulated. EGFR ligand-induced growth of BxPC3 cells can be measured by comparing the growth of the cells in the absence and presence of the ligand. The preferred EGFR ligand for measuring EGFR ligand-induced growth of BxPC3 or BxPC3-luc2 cells is EGF. In certain aspects, the ligand-induced growth is measured using saturating amounts of ligand. In certain aspects, EGF is used in an amount of 100 ng/ml of culture medium. In certain aspects, said EGF is the EGF R&D systems, cat. nr. 396-HB and 236-EG (see also WO2017/069628; which is incorporated by reference herein).

In certain aspects, an EGFR antibody as disclosed herein inhibits EGFR ligand induced growth of BxPC3 cells (ATCC CRL-1687) or BxPC3-luc2 cells (Perkin Elmer 125058). EGFR can bind a number of ligands and stimulate growth of the mentioned BxPC3 cells or BxPC3-luc2 cells. In the presence of a ligand the growth of BxPC3 or BxPC3-luc2 cells is stimulated. EGFR ligand-induced growth of BxPC3 cells can be measured by comparing the growth of the cells in the absence and presence of the ligand. In certain aspects, the EGFR ligand for measuring EGFR ligand-induced growth of BxPC3 or BxPC3-luc2 cells is EGF. In certain aspects, the ligand-induced growth is measured using saturating amounts of ligand. In certain aspects, EGF is used in an amount of 100 ng/ml of culture medium. In certain aspects, EGF is the EGF of R&D systems, cat. nr. 396-HB and 236-EG (see also WO2017/069628; which is incorporated by reference herein).

For the avoidance of doubt the reference to the growth of a cell as used herein refers to a change in the number of cells. Inhibition of growth refers to a reduction in the number of cells that would otherwise have been obtained. Increase in growth refers to an increase in the number of cells that would otherwise have been obtained. The growth of a cell typically refers to the proliferation of the cell.

Whether an antibody as described herein inhibits signaling or inhibits growth in a multispecific format is in certain aspects determined by the method as described herein above using a monospecific monovalent or monospecific bivalent version of the antibody. In certain aspects, such an antibody has binding sites for the receptor of which signaling is to be determined. A monospecific monovalent antibody can have a variable domain with an irrelevant binding specificity such as tetanus toxoid specificity. In certain aspects, said antibody is a bivalent monospecific antibody wherein the antigen binding variable domains consist of variable domains that bind the EGF-receptor family member.

In its Biclonics® antibody program, Merus has developed multispecific antibodies that target EGFR and LGR5 (Leucine-rich repeat containing G protein-coupled receptor). The efficacy of such multispecific antibodies has been assessed in vitro and in vivo using patient-derived CRC organoids and mice PDX models, respectively (see, e.g., WO2017/069628; which is incorporated by reference herein). Multispecific antibodies that target EGFR and LGR5 were shown to inhibit tumor growth. The potency of such inhibitory antibodies was shown to be correlated with the levels of LGR5 RNA expression by cells from the cancer. In certain aspects, said multispecific antibodies that target EGFR and LGR5 are as described in WO2017/069628.

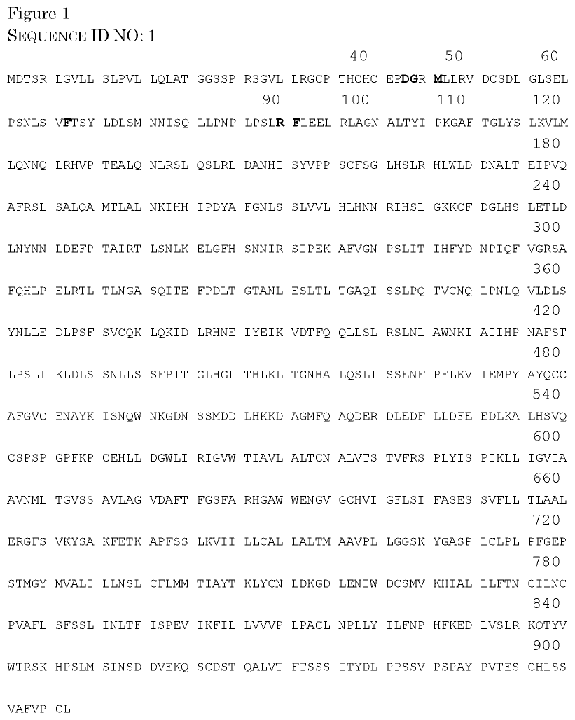

An antibody or a functional part, derivative and/or analogue thereof as described herein comprises a variable domain that binds an extracellular part of LGR5. In certain aspects, the variable domain that binds an extracellular part of LGR5 binds an epitope that is located within amino acid residues 21-118 of the sequence of FIG. 1 of which amino acid residues D43; G44, M46, F67, R90, and F91 are involved in binding of the antibody to the epitope.

In certain aspects, the LGR5 variable domain is a variable domain wherein one or more of the amino acid residue substitutions in LGR5 of D43A; G44A, M46A, F67A, R90A, and F91A reduces the binding of the variable domain to LGR5.

In certain aspects, the epitope on an extracellular part of LGR5 is located within amino acid residues 21-118 of the sequence of FIG. 1. In certain aspects, it is an epitope wherein the binding of the LGR5 variable domain to LGR5 is reduced by one or more of the following amino acid residue substitutions D43A; G44A, M46A, F67A, R90A, and F91A in LGR5.

The disclosure further provides an antibody with a variable domain that binds an extracellular part of EGFR and a variable domain that binds an extracellular part of LGR5 wherein the LGR5 variable domain binds an epitope on LGR5 that is located within amino acid residues 21-118 of the sequence of Figure. 1 In certain aspects, the epitope on LGR5 is a conformational epitope. In certain aspects, the epitope is located within amino acid residues 40-95 of the sequence of FIG. 1. In certain aspects, the binding of the antibody to LGR5 is reduced with one or more of the following amino acid residue substitutions D43A; G44A, M46A, F67A, R90A, and F91A.

Without being bound by theory it is believed that M46, F67, R90, and F91 of LGR5 as depicted in FIG. 1, are contact residues for a variable domain as indicated herein above, i.e. the antigen-binding site of a variable domain that binds the LGR5 epitope. That amino acid residue substitution D43A and G44A reduces the binding of an antibody can be due to the fact that these are also contact residues, however, it is also possible that these amino acid residue substitutions induce a (slight) modification of the conformation of the part of LGR5 that has one or more of the other contact residues (i.e. at positions 46, 67, 90 or 91) and that conformation change is such that antibody binding is reduced. The epitope is characterized by the mentioned amino acid substitutions. Whether an antibody binds the same epitope can be determined in various ways. In an exemplary method, CHO cells express LGR5 on the cell membrane, or an alanine substitution mutant, such as a mutant comprising one or more of the substitutions M46A, F67A, R90A, or F91A. A test antibody is contacted with the CHO cells and binding of the antibody to the cells compared. A test antibody binds the epitope if it binds to LGR5 and to a lesser extent to an LGR5 with a M46A, F67A, R90A, or F91A substitution. Comparing binding with a panel of mutants each comprising one alanine residue substitution is preferred. Such binding studies are well known in the art. Often the panel comprises single alanine substitution mutants covering essentially all amino acid residues. For LGR5 the panel only needs to cover the extracellular part of the protein and a part that warrants association with the cell membrane of course, when cells are used. Expression of a particular mutant can be compromised but this is easily detected by one or more LGR5 antibodies that bind to different region(s). If expression is also reduced for these control antibodies the level or folding of the protein on the membrane is compromised for this particular mutant. Binding characteristics of the test antibody to the panel readily identifies whether the test antibodies exhibit reduced binding to mutants with a M46A, F67A, R90A, or F91A substitution and thus whether the test antibody is an antibody of the invention. Reduced binding to mutants with a M46A, F67A, R90A, or F91A substitution also identifies the epitope to be located within amino acid residues 21-118 of the sequence of FIG. 1. In certain aspects, the panel includes a D43A substitution mutant; a G44A substitution mutant of both. The antibody with the VH sequence of the VH of MF5816 exhibits reduced binding to these substitution mutants.

Without being bound by any theory it is believed that amino acid residues 1462; G465; K489; 1491; N493; and C499 as depicted FIG. 2 are involved in binding an epitope by an antibody comprising a variable domain as indicated herein above. In certain aspects, involvement in binding is determined by observing a reduced binding of the variable domain to an EGFR with one or more of the amino acid residue substitutions selected from I462A; G465A; K489A; I491A; N493A; and C499A. In an exemplary method, CHO cells express EGFR on the cell membrane, or an alanine substitution mutant, such as a mutant comprising one or more of the substitutions selected from I462A; G465A; K489A; I491A; N493A; and C499A. A test antibody is contacted with the CHO cells and binding of the antibody to the cells compared. A test antibody binds the epitope if it binds to EGFR and to a lesser extent to an EGFR with a I462A; G465A; K489A; 1491A; N493A; and C499A substitution. Comparing binding with a panel of mutants each comprising one alanine residue substitution is preferred. Such binding studies are well known in the art. Often the panel comprises single alanine substitution mutants covering essentially all amino acid residues. For EGFR the panel only needs to cover the extracellular part of the protein and a part that warrants association with the cell membrane of course, when cells are used. Expression of a particular mutant can be compromised but this is easily detected by one or more EGFR antibodies that bind to different region(s). If expression is also reduced for these control antibodies the level or folding of the protein on the membrane is compromised for this particular mutant. Binding characteristics of the test antibody to the panel readily identifies whether the test antibodies exhibit reduced binding to mutants with a I462A; G465A; K489A; I491A; N493A; and C499A substitution.

In one aspect, the variable domain that binds an epitope on an extracellular part of human EGFR is a variable domain that binds an epitope that is located within amino acid residues 420-480 of the sequence depicted in FIG. 2. In certain aspects, the binding of the variable domain to EGFR is reduced by one or more of the following amino acid residue substitutions I462A; G465A; K489A; I491A; N493A; and C499A in EGFR. In certain aspects, the binding of the antibody to human EGFR interferes with the binding of EGF to the receptor. In certain aspects, the epitope on EGFR is a conformational epitope. In one aspect the epitope is located within amino acid residues 420-480 of the sequence depicted in FIG. 2, such as within 430-480 of the sequence depicted in FIG. 2. In certain aspects, said epitope is located within 438-469 of the sequence depicted in FIG. 2.

Without being bound by theory it is believed that the contact residues of the epitope, i.e. where the variable domain contacts the human EGFR are 1462; K489; 1491; and N493. The amino acid residues G465 and C499 are likely indirectly involved in the binding of the antibody to EGFR.

In certain aspects, the variable domain that binds human EGFR, is a variable domain with a heavy chain variable region that comprises at least the CDR3 sequence of the VH of MF3755 as depicted in FIG. 3 or a CDR3 sequence that differs in at most three, or in at most two, or in no more than one amino acid from a CDR3 sequence of the VH of MF3755 as depicted in FIG. 3.

In certain aspects, the variable domain that binds human EGFR, is a variable domain with a heavy chain variable region that comprises at least the CDR1, CDR2 and CDR3 sequences of the VH of MF3755 as depicted in FIG. 3; or the CDR1, CDR2 and CDR3 sequences of the VH of MF3755 as depicted in FIG. 3 with at most three, or at most two, or at most one amino acid substitutions.

In certain aspects, the variable domain that binds human EGFR, is a variable domain with a heavy chain variable region that comprises the sequence of the VH chain of MF3755 as depicted in FIG. 3; or the amino acid sequence of the VH chain of MF3755 depicted in FIG. 3 having at most 15 (or in certain aspects 1, 2, 3, 4, 5, 6, 7, 8, 9 or 10, or in certain aspects 1, 2, 3, 4 or 5) amino acid insertions, deletions, substitutions or a combination thereof with respect to the VH chain of MF3755.

In certain aspects, the disclosure provides an antibody comprising a variable domain that binds an extracellular part of EGFR and a variable domain that binds an extracellular part of LGR5,
wherein a heavy chain variable region of said variable domain comprises at least the CDR3 sequence of an EGFR specific heavy chain variable region selected from the group consisting of MF3370; MF3755; MF4280 or MF4289 as depicted in FIG. 3 or wherein a heavy chain variable region of said variable domain comprises a heavy chain CDR3 sequence that differs in at most three, or in at most two, or in no more than one amino acid from a CDR3 sequence of a VH selected from the group consisting of MF3370; MF3755; MF4280 or MF4289 as depicted in FIG. 3. In certain aspects, said variable domain comprises a heavy chain variable region comprising at least the CDR3 sequence of MF3370; MF3755; MF4280 or MF4289 as depicted in FIG. 3.

In certain aspects, said variable domain comprises a heavy chain variable region comprising at least the CDR1, CDR2 and CDR3 sequences of an EGFR specific heavy chain variable region selected from the group consisting of MF3370; MF3755; MF4280 or MF4289 as depicted in FIG. 3, or heavy chain variable region comprising at least CDR1, CDR2 and CDR3 sequences that differ in at most three, or in at most two, or in at most one amino acid from the CDR1, CDR2 and CDR3 sequences of an EGFR specific heavy chain variable region selected from the group consisting of MF3370; MF3755; MF4280 or MF4289 as depicted in FIG. 3. In certain aspects, said variable domain comprises a heavy chain variable region comprising at least the CDR1, CDR2 and CDR3 sequences of MF3370; MF3755; MF4280 or MF4289 as depicted in FIG. 3. In certain aspects, the heavy chain variable region is MF3755. In certain aspects, the heavy chain variable region is MF4280.

In certain aspects, the antibody comprising a variable domain that binds an extracellular part of EGFR and a variable domain that binds an extracellular part of LGR5, wherein the EGFR binding variable domain has a CDR3, a CDR1, CDR2, and CDR3 and/or a VH sequence as indicated herein above, and the variable domain that binds LGR5 comprises at least the CDR3 sequence of an LGR5 specific heavy chain variable region selected from the group consisting of MF5790; MF5803; MF5805; MF5808; MF5809; MF5814; MF5816; MF5817; or MF5818 as depicted in FIG. 3 or a heavy chain CDR3 sequence that differs in at most three, or in at most two, or in no more than one amino acid from a CDR3 sequence of a VH selected from the group consisting of MF5790; MF5803; MF5805; MF5808; MF5809; MF5814; MF5816; MF5817; or MF5818 as depicted in FIG. 3. In certain aspects, said variable domain comprises a heavy chain variable region comprising at least the CDR3 sequence of MF5790; MF5803; MF 5805; MF5808; MF5809; MF5814; MF5816; MF5817; or MF5818 as depicted in FIG. 3.

In certain aspects, the LGR5 variable domain comprises a heavy chain variable region comprising at least the CDR1, CDR2 and CDR3 sequences of an LGR5 specific heavy chain variable region selected from the group consisting of MF5790; MF5803; MF5805; MF5808; MF5809; MF5814; MF5816; MF5817; or MF5818 as depicted in FIG. 3, or heavy chain CDR1, CDR2 and CDR3 sequences that differ in at most three, or in at most two, or in at most one amino acid from the CDR1, CDR2 and CDR3 sequences of LGR5 specific heavy chain variable region selected from the group consisting of MF5790; MF5803; MF5805; MF5808; MF5809; MF5814; MF5816; MF5817; or MF5818 as depicted in FIG. 3. In certain aspects, said variable domain comprises a heavy chain variable region comprising at least the CDR1, CDR2 and CDR3 sequences of MF5790; MF5803; MF5805; MF5808; MF5809; MF5814; MF5816; MF5817; or MF5818 as depicted in FIG. 3. In certain aspects, the heavy chain variable regions are MF5790; MF5803; MF5814; MF5816; MF5817; or MF5818. In certain aspects, the heavy chain variable regions are MF5790; MF5814; MF5816; and MF5818. In certain aspects, the heavy chain variable region is MF5814, MF5818 or MF5816. In certain aspects, the heavy chain variable region is MF5816. In certain aspects, the heavy chain variable region is MF5818.

It has been shown that the antibodies comprising one or more variable domains with a heavy chain variable region MF3755 or one or more CDRs thereof have a better effectivity when used to inhibit growth of an EGFR ligand responsive cancer or cell. In the context of bispecific or multispecific antibodies, an arm of the antibody comprising a variable domain with a heavy chain variable region MF3755 or one or more CDRs thereof combines well with an arm comprising a variable domain with a heavy chain variable region MF5818 or one or more CDRs thereof.

VH chains of variable domains that bind EGFR or LGR5 can have one or more amino acid substitutions with respect to the sequence depicted in FIG. 3. In certain aspects, a VH chain has an amino acid sequence of an EGFR or LGR5 VH of FIG. 3, having at most 15, or 1, 2, 3, 4, 5, 6, 7, 8, 9 or 10 and, in certain aspects, having 1, 2, 3, 4 or 5 amino acid insertions, deletions, substitutions or a combination thereof with respect to the VH chain sequence of FIG. 3.

CDR sequences can have one or more amino acid residue substitutions with respect to a CDR sequence in the figures. Such one or more substitutions are for instance made for optimization purposes, such as to improve binding strength or the stability of the antibody. Optimization is for instance performed by mutagenesis procedures where after the stability and/or binding affinity of the resulting antibodies are preferably tested and an improved EGFR specific CDR sequence or LGR5 specific CDR sequence is preferably selected. A skilled person is well capable of generating antibody variants comprising at least one altered CDR sequence according to the invention. For instance, conservative amino acid substitution may be applied. Examples of conservative amino acid substitution include the substitution of one hydrophobic residue such as isoleucine, valine, leucine or methionine for another hydrophobic residue, and the substitution of one polar residue for another polar residue, such as the substitution of arginine for lysine, glutamic acid for aspartic acid, or glutamine for asparagine.

In certain aspects, the mentioned at most 15 (or in certain aspects, 1, 2, 3, 4, 5, 6, 7, 8, 9 or 10 or in certain aspects 1, 2, 3, 4 or 5) amino acid substitutions in a VH or VL as specified herein are conservative amino acid substitutions. In certain aspects, the amino acid insertions, deletions and substitutions in a VH or VL as specified herein are not present in the CDR3 region. In certain aspects, the mentioned amino acid insertions, deletions and substitutions are also not present in the CDR1 and CDR2 regions. In certain aspects, the mentioned amino acid insertions, deletions and substitutions are also not present in the FR4 region.

In certain aspects, the mentioned at most 15 (or in certain aspects, 1, 2, 3, 4, 5, 6, 7, 8, 9 or 10, or in certain aspects 1, 2, 3, 4 or 5) amino acid substitutions are conservative amino acid substitutions. In certain aspects, the insertions, deletions, substitutions or a combination thereof are not in the CDR3 region of the VH chain, in certain aspects, not in the CDR1, CDR2 or CDR3 region of the VH chain and in certain aspects, not in the FR4 region.

In certain aspects, the treatment of the present disclosure makes use of an antibody comprising a variable domain that binds an extracellular part of EGFR and in certain aspects a variable domain that binds an extracellular part of LGR5 which comprises
the amino acid sequence of VH chain MF3755 as depicted in FIG. 3; or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH; and
wherein the VH chain of the variable domain that binds LGR5 comprises
the amino acid sequence of VH chain MF5790 as depicted in FIG. 3; or
the amino acid sequence of VH chain MF5790 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH.

In certain aspects, the treatment of the present disclosure makes use of an antibody comprising a variable domain that binds an extracellular part of EGFR and a variable domain that, in certain aspects, binds an extracellular part of LGR5 comprises
the amino acid sequence of VH chain MF3755 as depicted in FIG. 3; or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH; and
wherein the VH chain of the variable domain that binds LGR5 comprises
the amino acid sequence of VH chain MF5803 as depicted in FIG. 3; or
the amino acid sequence of VH chain MF5803 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH.

In certain aspects, the treatment of the present disclosure makes use of an antibody comprising a variable domain that binds an extracellular part of EGFR and a variable domain that, in certain aspects, binds an extracellular part of LGR5 comprises
the amino acid sequence of VH chain MF3755 as depicted in FIG. 3; or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH; and
wherein the VH chain of the variable domain that binds LGR5 comprises
the amino acid sequence of VH chain MF5814 as depicted in FIG. 3; or
the amino acid sequence of VH chain MF5814 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH.

In certain aspects, the treatment of the present disclosure makes use of an antibody comprising a variable domain that binds an extracellular part of EGFR and a variable domain that, in certain aspects, binds an extracellular part of LGR5 comprises
the amino acid sequence of VH chain MF3755 as depicted in FIG. 3; or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH; and
wherein the VH chain of the variable domain that binds LGR5 comprises
the amino acid sequence of VH chain MF5816 as depicted in FIG. 3; or
the amino acid sequence of VH chain MF5816 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH.

In certain aspects, the treatment of the present disclosure makes use of an antibody comprising a variable domain that binds an extracellular part of EGFR and a variable domain that, in certain aspects, binds an extracellular part of LGR5 comprises
the amino acid sequence of VH chain MF3755 as depicted in FIG. 3; or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH; and
wherein the VH chain of the variable domain that binds LGR5 comprises
the amino acid sequence of VH chain MF5817 as depicted in FIG. 3; or
the amino acid sequence of VH chain MF5817 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH.

In certain aspects, the treatment of the present disclosure makes use of an antibody comprising a variable domain that binds an extracellular part of EGFR and a variable domain that, in certain aspects, binds an extracellular part of LGR5 comprises
the amino acid sequence of VH chain MF3755 as depicted in FIG. 3 or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH; and
wherein the VH chain of the variable domain that binds LGR5 comprises
the amino acid sequence of VH chain MF5818 as depicted in FIG. 3; or
the amino acid sequence of VH chain MF5818 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect said VH.

In certain aspects, the variable domain that binds an extracellular part of EGFR comprises a heavy chain variable region comprising the CDR1, CDR2 and CDR3 sequences of a variable region selected from the group consisting of MF3370; MF3755; MF4280 or MF4289 as depicted in FIG. 3 and wherein the variable domain that binds an extracellular part of LGR5 comprises a heavy chain variable region comprising the CDR1, CDR2 and CDR3 sequences of a variable region selected from the group consisting of MF5790; MF5803; MF5805; MF5808; MF5809; MF5814; MF5816; MF5817; or MF5818 as depicted in FIG. 3.

In certain aspects, the variable domain that binds an extracellular part of EGFR comprises a heavy chain variable region comprising the CDR1, CDR2 and CDR3 sequences of the variable region of MF3755 as depicted in FIG. 3 and wherein the variable domain that binds an extracellular part of LGR5 comprises a heavy chain variable region comprising the CDR1, CDR2 and CDR3 sequences of the variable region of MF5816 as depicted in FIG. 3.

In certain aspects, a VH chain of the variable domain that binds EGFR comprises the amino acid sequence of VH chain MF3370; MF3755; MF4280 or MF4289 as depicted in FIG. 3; or the amino acid sequence of VH chain MF3370; MF3755; MF4280 or MF4289 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH; and wherein a VH chain of the variable domain that binds LGR5 comprises the amino acid sequence of VH chain MF5790; MF5803; MF5805; MF5808; MF5809; MF5814; MF5816; MF5817; or MF5818 as depicted in FIG. 3; or the amino acid sequence of VH chain MF5790; MF5803; MF5805; MF5808; MF5809; MF5814; MF5816; MF5817; or MF5818 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH.

In certain aspects, a VH chain of the variable domain that binds EGFR comprises the amino acid sequence of VH chain MF3755 as depicted in FIG. 3; or the amino acid sequence of VH chain MF3755 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH; and wherein a VH chain of the variable domain that binds LGR5 comprises the amino acid sequence of VH chain MF5816 as depicted in FIG. 3; or the amino acid sequence of VH chain MF5816 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH.

In certain aspects, a VH chain of the variable domain that binds EGFR comprises the amino acid sequence of VH chain MF3370; MF3755; MF4280 or MF4289 as depicted in FIG. 3; and wherein a VH chain of the variable domain that binds LGR5 comprises the amino acid sequence of VH chain MF5790; MF5803; MF5805; MF5808; MF5809; MF5814; MF5816; MF5817; or MF5818 as depicted in FIG. 3.

In certain aspects, a VH chain of the variable domain that binds EGFR comprises the amino acid sequence of VH chain MF3755 as depicted in FIG. 3; and wherein a VH chain of the variable domain that binds LGR5 comprises the amino acid sequence of VH chain MF5816 as depicted in FIG. 3.

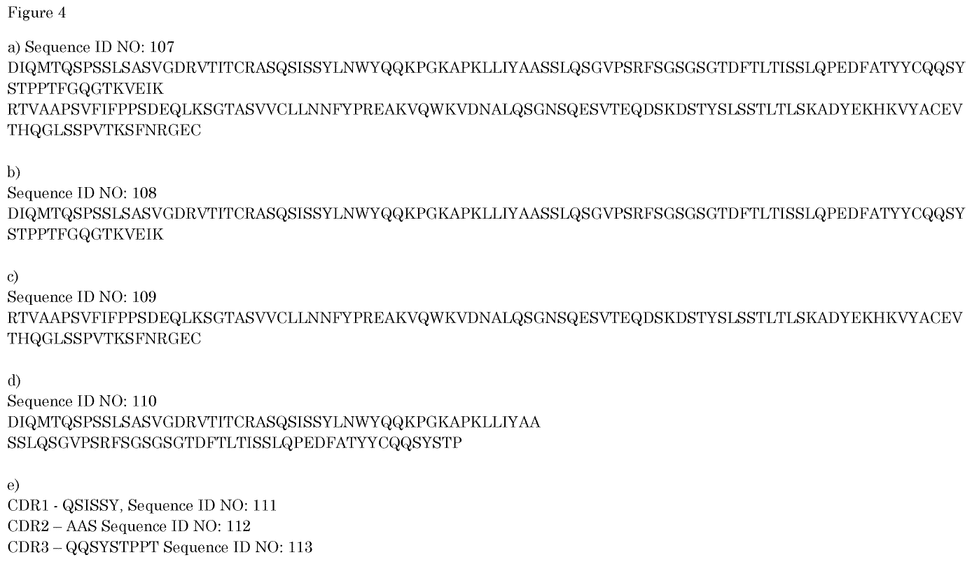

In certain aspects, both said variable domains that bind EGFR and that bind LGR5 comprise the CDR1, CDR2 and CDR3 regions of the light chain variable region as depicted in FIG. 4b.

In certain aspects, both said variable domains that bind EGFR and that bind LGR5 comprise the light chain variable region as depicted in FIG. 4b, which variable light chain region comprises from 0 to 10 amino acid insertions, deletions, substitutions, additions or a combination thereof, wherein the amino acid insertions, deletions and substitutions are not present in the CDR1, CDR2 and CDR3 light chain variable regions.

Additional variants of the disclosed amino acid sequences which retain EGFR or LGR5 binding can be obtained, for example, from phage display libraries which contain the rearranged human IGKV1-39/IGKJ1 VL region (De Kruif et al. Biotechnol Bioeng. 2010 (106)741-50), and a collection of VH regions incorporating amino acid substitutions into the amino acid sequence of an EGFR or LGR5 VH region disclosed herein, as previously described (e.g., WO2017/069628). Phages encoding Fab regions which bind EGFR or LGR5 may be selected and analyzed by flow cytometry, and sequenced to identify variants with amino acid substitutions, insertions, deletions or additions which retain antigen binding.

The light chain variable regions of the VH/VL EGFR and LGR5 variable domains of the EGFR/LGR5 antibody may be the same or different. In certain aspects, the VL region of the VH/VL EGFR variable domain of the EGFR/LGR5 antibody is similar to the VL region of the VH/VL LGR5 variable domain. In certain aspects, VL regions in the VH/VL variable domains that bind EGFR and LGR5 are identical.

In certain aspects, the light chain variable region of one or both VH/VL variable domains of the EGFR/LGR5 antibody comprises a common light chain variable region. In certain aspects, the common light chain variable region of one or both VH/VL variable domains comprises a germline IgVκ1-39 variable region V-segment. In certain aspects, the light chain variable region of one or both VH/VL variable domains comprises the kappa light chain V-segment IgVκ1-39*01. IgVκ1-39 is short for Immunoglobulin Variable Kappa 1-39 Gene. The gene is also known as Immunoglobulin Kappa Variable 1-39; IGKV139; IGKV1-39. External Ids for the gene are HGNC: 5740; Entrez Gene: 28930; Ensembl: ENSG00000242371. The amino acid sequence for a suitable V-region is provided in FIG. 4. The V-region can be combined with one of five J-regions. In certain aspects, the J-regions are jk1 and jk5, and the joined sequences are indicated as IGKV1-39/jk1 and IGKV1-39/jk5; alternative names are IgVκ1-39*01/IGJκ1*01 or IgVκ1-39*01/IGJκ5*01 (nomenclature according to the IMGT database worldwide web at imgt.org). In certain aspects, the light chain variable region of one or both VH/VL variable domains comprises the kappa light chain IgVκ1-39*01/IGJκ1*01 or IgVκ1-39*01/IGJκ1*05 (described in FIG. 4).

In certain aspects, the light chain variable region of FIG. 4d) comprises LCDR1, LCDR2 and LCDR sequences. Such sequences can be determined or annotated by the skilled person using for instance an annotation system like IMGT, Chothia, Kabat or other suitable annotation systems. In certain aspects, said light chain variable region is comprises by one or both VH/VL variable domains and present in an EGFR binding antibody of the present disclosure and comprise LCDR1, LCDR2 and LCDR sequences. Such sequences can be determined or annotated by the skilled person using for instance an annotation system like IMGT, Chothia, Kabat or other suitable annotation systems.

In certain aspects, the light chain variable region of one or both VH/VL variable domains of the EGFR/LGR5 bispecific antibody comprises an LCDR1 comprising the amino acid sequence QSISSY (described in FIG. 4), an LCDR2 comprising the amino acid sequence AAS (described in FIG. 4), and an LCDR3 comprising the amino acid sequence QQSYSTP (described in FIG. 4). In certain aspects, these CDRs are according to IMGT. In certain aspects, the light chain variable region of one or both VH/VL variable domains of the EGFR/LGR5 antibody comprises an LCDR1 comprising the amino acid sequence QSISSY, an LCDR2 comprising the amino acid sequence AASSLQS, and an LCDR3 comprising the amino acid sequence QQSYSTP.

In certain aspects, the light chain variable region of one or both VH/VL variable domains of the EGFR/LGR5 bispecific antibody comprises an LCDR1 comprising the amino acid sequence QSISSY (described in FIG. 4), an LCDR2 comprising the amino acid sequence AAS (described in FIG. 4), and an LCDR3 comprising the amino acid sequence QQSYSTPPT, i.e. the CDRs according to IMGT (as described in FIG. 4). In certain aspects, the light chain variable region of one or both VH/VL variable domains of the EGFR/LGR5 antibody comprises an LCDR1 comprising the amino acid sequence QSISSY, an LCDR2 comprising the amino acid sequence AASSLQS, and an LCDR3 comprising the amino acid sequence QQSYSTPPT.

In certain aspects, one or both VH/VL variable domains of the EGFR/LGR5 antibody comprise a light chain variable region comprising an amino acid sequence that is at least 90%, in certain aspects at least 95%, in certain aspects at least 97%, in certain aspects at least 98%, in certain aspects at least 99% identical or in certain aspects 100% identical to the amino acid sequence of set forth in FIG. 4. In certain aspects, one or both VH/VL variable domains of the EGFR/LGR5 antibody comprise a light chain variable region comprising an amino acid sequence that is at least 90%, in certain aspects at least 95%, in certain aspects at least 97%, in certain aspects at least 98%, in certain aspects at least 99% identical or in certain aspects 100% identical to the amino acid sequence of set forth in FIG. 4.

For example, the variable light chain of one or both VH/VL variable domains of the EGFR/LGR5 antibody can have from 0 to 10, or in certain aspects from 0 to 5 amino acid insertions, deletions, substitutions, additions or a combination thereof with respect to a sequence in FIG. 4. In certain aspects, the light chain variable region of one or both VH/VL variable domains of the EGFR/LGR5 antibody comprises from 0 to 9, from 0 to 8, from 0 to 7, from 0 to 6, from 0 to 5, from 0 to 4, in certain aspects from 0 to 3, in certain aspects from 0 to 2, in certain aspects from 0 to 1 and in certain aspects 0 amino acid insertions, deletions, substitutions, additions with respect to the indicated amino acid sequence, or a combination thereof.

Also, the light chain variable region of one or both VH/VL variable domains of the EGFR/LGR5 antibody may comprise the amino acid sequence of a sequence as depicted in FIG. 4. In certain aspects, both VH/VL variable domains of the EGFR/LGR5 antibody comprise identical VL regions. In certain aspects, the VL of both VH/VL variable domains of the EGFR/LGR5 bispecific antibody comprises the amino acid sequence set forth in FIG. 4.

In certain aspects, the EGFR/LGR5 antibody as described herein is a bispecific antibody having two variable domains, one that binds EGFR and another that binds LGR5 as described herein. EGFR/LGR5 bispecific antibodies for use in the methods disclosed herein can be provided in a number of formats. Many different formats of bispecific antibodies are known in the art, and have been reviewed by Kontermann (Drug Discov Today, 2015 July; 20(7):838-47; MAbs, 2012 March-April; 4(2):182-97) and in Spiess et al., (Alternative molecular formats and therapeutic applications for bispecific antibodies. Mol. Immunol. (2015) http: //dx.doi.org/10.1016/j.molimm.2015.01.003), which are each incorporated herein by reference. For example, bispecific antibody formats that are not classical antibodies with two VH/VL combinations, have at least a variable domain comprising a heavy chain variable region and a light chain variable region. This variable domain may be linked to a single chain Fv-fragment, monobody, a VH and a Fab-fragment that provides the second binding activity.

In certain aspects, the EGFR/LGR5 bispecific antibodies used in the methods provided herein are generally of the human IgG subclass (e.g., for instance IgG1, IgG2, IgG3, IgG4). In certain aspects, the antibodies are of the human IgG1 subclass. Full length IgG antibodies are preferred because of their favorable half-life and for reasons of low immunogenicity. Accordingly, the EGFR/LGR5 bispecific antibody is in certain aspects, a full length IgG molecule. In certain aspects, the EGFR/LGR5 bispecific antibody is a full length IgG1 molecule.

Accordingly, in certain aspects, the EGFR/LGR5 bispecific antibody comprises a fragment crystallizable (Fc). In certain aspects, the Fc of the EGFR/LGR5 bispecific antibody is comprised of a human constant region. A constant region or Fc of the EGFR/LGR5 bispecific antibody may contain one or more, or not more than 10, or not more than 5 amino-acid differences with a constant region of a naturally occurring human antibody. For example, each Fab-arm of the bispecific antibodies may further include an Fc-region comprising modifications promoting the formation of the bispecific antibody, promoting stability and/or other features described herein.

Bispecific antibodies are typically produced by cells that express nucleic acid(s) encoding the antibody. Accordingly, in certain aspects, the bispecific EGFR/LGR5 antibodies disclosed herein are produced by providing a cell comprising one or more nucleic acids that encode the heavy and light chain variable regions and constant regions of the bispecific EGFR/LGR5 antibody. In certain aspects, the cell is an animal cell, such as a mammal cell, or a primate cell and in certain aspects a human cell. A suitable cell is any cell capable of comprising and preferably of producing the EGFR/LGR5 bispecific antibody.

Suitable cells for antibody production are known in the art and include a hybridoma cell, a Chinese hamster ovary (CHO) cell, an NS0 cell or a PER-C6 cell. Various institutions and companies have developed cell lines for the large scale production of antibodies, for instance for clinical use. Non-limiting examples of such cell lines are CHO cells, NS0 cells or PER.C6 cells. In particular, said cell is a human cell. Preferably a cell is transformed by an adenovirus E1 region or a functional equivalent thereof. A preferred example of such a cell line is the PER.C6 cell line or equivalent thereof. In a particular, said cell is a CHO cell or a variant thereof. Preferably the variant makes use of a Glutamine synthetase (GS) vector system for expression of an antibody. In certain aspects, the cell is a CHO cell.

In certain aspects, the cell expresses the different light and heavy chains that make up the EGFR/LGR5 bispecific antibody. In certain aspects, the cell expresses two different heavy chains and at least one light chain. In certain aspects, the cell expresses a “common light chain” as described herein to reduce the number of different antibody species (combinations of different heavy and light chains). For example, the respective VH regions are cloned into expression vectors using methods known in the art for production of bispecific IgG (WO2013/157954; incorporated herein by reference), in conjunction with the rearranged human IGKV1-39/IGKJ1 (huVκ1 39) light chain, previously shown to be able to pair with more than one heavy chain thereby giving rise to antibodies with diverse specificities, which facilitates the generation of bispecific molecules (De Kruif et al. J. Mol. Biol. 2009 (387) 548 58; WO2009/157771).

An antibody producing cell that expresses a common light chain and equal amounts of the two heavy chains typically produces 50% bispecific antibody and 25% of each of the monospecific antibodies (i.e. having identical heavy light chain combinations). Several methods have been published to favor the production of the bispecific antibody over the production of the respective monospecific antibodies. Such is typically achieved by modifying the constant region of the heavy chains such that they favor heterodimerization (i.e. dimerization with the heavy chain of the other heavy/light chain combination) over homodimerization. In certain aspects, the bispecific antibody of the invention comprises two different immunoglobulin heavy chains with compatible heterodimerization domains. Various compatible heterodimerization domains have been described in the art. In certain aspects, the compatible heterodimerization domains are compatible immunoglobulin heavy chain CH3 heterodimerization domains. The art describes various ways in which such hetero-dimerization of heavy chains can be achieved.

A preferred method for producing the EGFR/LGR5 bispecific antibody is disclosed in U.S. Pat. Nos. 9,248,181 and 9,358,286. Specifically, preferred mutations to produce essentially only bispecific full length IgG molecules are the amino acid substitutions L351K and T366K (EU numbering) in the first CH3 domain (the ‘KK-variant’ heavy chain) and the amino acid substitutions L351D and L368E in the second domain (the ‘DE-variant’ heavy chain), or vice versa. As previously described, the DE-variant and KK-variant preferentially pair to form heterodimers (so-called ‘DEKK’ bispecific molecules). Homodimerization of DE-variant heavy chains (DEDE homodimers) or KK-variant heavy chains (KKKK homodimers) hardly occurs due to strong repulsion between the charged residues in the CH3-CH3 interface between identical heavy chains.

Accordingly, in certain aspects, the heavy chain/light chain combination that comprises the variable domain that binds EGFR, comprises a DE variant of the heavy chain. In certain aspects, the heavy chain/light chain combination that comprises the variable domain that binds LGR5 comprises a KK variant of the heavy chain.

A candidate EGFR/LGR5 IgG bispecific antibody can be tested for binding using any suitable assay. For example, binding to membrane-expressed EGFR or LGR5 on CHO cells can be assessed by flow cytometry (according to the FACS procedure as previously described in WO2017/069628). In certain aspects, the binding of a candidate EGFR/LGR5 bispecific antibody to LGR5 on CHO cells is demonstrated by flow cytometry, performed according to standard procedures known in the art. Binding to the CHO cells is compared with CHO cells that have not been transfected with expression cassettes for EGFR and/or LGR5. The binding of the candidate bispecific IgG1 to EGFR is determined using CHO cells transfected with an EGFR expression construct; a LGR5 monospecific antibody and an EGFR monospecific antibody, as well as an irrelevant IgG1 isotype control mAb are included in the assay as controls (e.g., an antibody which binds LGR5 and another antigen such as tetanus toxin (TT)).

The affinities of the LGR5 and EGFR Fabs of a candidate EGFR/LGR5 bispecific antibody for their targets can be measured by surface plasmon resonance (SPR) technology using a BIAcore T100. Briefly, an anti-human IgG mouse monoclonal antibody (Becton and Dickinson, cat. Nr. 555784) is coupled to the surfaces of a CM5 sensor chip using free amine chemistry (NHS/EDC). Then the bsAb is captured onto the sensor surface. Subsequently, the recombinant purified antigens human EGFR (Sino Biological Inc, cat. Nr. 11896-H07H) and human LGR5 protein are run over the sensor surface in a concentration range to measure on- and off-rates. After each cycle, the sensor surface is regenerated by a pulse of HCl and the bsAb is captured again. From the obtained sensorgrams, on- and off-rates and affinity values for binding to human LGR5 and EGFR are determined using the BIAevaluation software, as previously described for CD3 in US 2016/0368988.

An antibody as disclosed herein is typically a bispecific full length antibody, in certain aspects of the human IgG subclass. In certain aspects, said antibody is of the human IgG1 subclass. Such antibodies have good ADCC properties which can, if desired, be enhanced by techniques known in the art, have favorable half-life upon in vivo administration to humans and CH3 engineering technology exists that can provide for modified heavy chains that preferentially form heterodimers over homodimers upon co-expression in clonal cells.

ADCC activity of an antibody can be improved when the antibody itself has a low ADCC activity, by modifying the constant region of the antibody. Another way to improve ADCC activity of an antibody is by enzymatically interfering with the glycosylation pathway resulting in a reduced fucose. Several in vitro methods exist for determining the efficacy of antibodies or effector cells in eliciting ADCC. Among these are chromium-51 [Cr51] release assays, europium [Eu] release assays, and sulfur-35 [S35] release assays. Usually, a labeled target cell line expressing a certain surface-exposed antigen is incubated with antibody specific for that antigen. After washing, effector cells expressing Fc receptor CD16 are co-incubated with the antibody-labeled target cells. Target cell lysis is subsequently measured by release of intracellular label by a scintillation counter or spectrophotometry.

A bispecific antibody as disclosed herein can be ADCC enhanced. In certain aspects, such a bispecific antibody is afucosylated. In certain aspects, a bispecific antibody comprises a reduced amount of fucosylation of the N-linked carbohydrate structure in the Fc region, when compared to the same antibody produced in a normal CHO cell. Low fucose levels are associated with increased CD16 (FcγRIIIa) binding on NK effector cells, resulting in increased ADCC activity. In certain aspects, and in addition to its direct antitumor activity, a bispecific antibody of the present disclosure can eliminate tumor cells following opsonization and subsequent natural killer (NK) cell-mediated ADCC activity and complement-dependent cytotoxic (CDC) activity.

The antibody that comprises a variable domain that binds an extracellular part of EGFR and a variable domain that binds an extracellular part of LGR5 may further comprise one or more additional variable domains that can bind one or more further targets. In certain aspects, further target is a protein, such as a membrane protein comprising an extracellular part. A membrane protein as used herein is a cell membrane protein, such as a protein that is in the outer membrane of a cell, the membrane that separates the cell from the outside world. The membrane protein has an extracellular part. A membrane protein is at least on a cell if it contains a transmembrane region that is in the cell membrane of the cell.

Antibodies with more than two variable domains are known in the art. For instance, it is possible to attach an additional variable domain. In certain aspects, an antibody with three or more variable domains is a multivalent multimer antibody as described in PCT/NL2019/050199 which is incorporated by reference herein.

In certain aspects, the antibody is a bispecific antibody comprising two variable domains, wherein one variable domain binds an extracellular part of EGFR and another variable domain binds an extracellular part of LGR5. In certain aspects, the variable domains are variable domains as described herein.

A functional part of an antibody as described herein comprises at least a variable domain that binds an extracellular part of EGFR and a variable domain that binds an extracellular part of LGR5 as described herein. It thus comprises the antigen binding parts of an antibody as described herein and typically contains the variable domains of the antibody. A variable domain of a functional part can be a single chain Fv-fragment or a so-called single domain antibody fragment. In certain aspects, the antibody parts or derivatives have at least two variable domains of an antibody or equivalents thereof. Non-limiting examples of such variable domains or equivalents thereof are F(ab)-fragments and Single chain Fv fragments. A functional part of a bispecific antibody comprises the antigen binding parts of the bispecific antibody, or a derivative and/or analogue of the binding parts. As mentioned herein above, the binding part of an antibody is encompassed in the variable domain.

In certain aspects, the antibody that comprises one variable domain which binds an extracellular part of EGFR is selected from amivantamab (Janssen Biotech), bafisontamab (EpimAb Biotherapeutics), REGN-7075 (Regeneron Pharmaceuticals Inc.), BCA-101 (Bicara Therapeutics), PM-1080 (Biotheus Inc.), duligotuzumab (Genentech, Inc.), AFM-24 (Affimed GmbH).

### Formulations and Pharmaceutical Compositions

In certain aspects, the treatment of the present disclosure makes use of an antibody or functional part, derivative and/or analogue thereof as disclosed herein (i.e., as a therapeutic agent) and a pharmaceutically acceptable carrier and an immune checkpoint inhibitor as disclosed herein (i.e., as a further therapeutic agent) and a pharmaceutically acceptable carrier. Such pharmaceutical compositions are useful in the treatment of cancer, in particular for the treatment of head and neck cancer. As used herein, the term “pharmaceutically acceptable” means approved by a government regulatory agency or listed in the U.S. Pharmacopeia or another generally recognized pharmacopeia for use in animals, particularly in humans, and includes any and all solvents, salts, dispersion media, coatings, antibacterial and antifungal agents, isotonic and absorption delaying agents, and the like that are physiologically compatible. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions. Liquid compositions for parenteral administration can be formulated for administration by injection or continuous infusion. Routes of administration by injection or infusion include intravesical, intratumoral, intravenous, intraperitoneal, intramuscular, intrathecal and subcutaneous. Depending on the route of administration (e.g., intravenously, subcutaneously, intra-articularly and the like) the active compound may be coated in a material to protect the compound from the action of acids and other natural conditions that may inactivate the compound.

Said pharmaceutical composition comprising said antibody, functional part, derivative and/or analogue thereof that comprises a variable domain that binds an extracellular part of EGFR may be contained in a holder that is separate, meaning not physically linked, from a holder that contains said pharmaceutical composition comprising said immune checkpoint inhibitor.

In certain aspects, said pharmaceutical composition comprising said antibody, functional part, derivative and/or analogue thereof that comprises a variable domain that binds an extracellular part of EGFR and LGR5 may be contained in a holder that is separate, meaning not physically linked, from a holder that contains said pharmaceutical composition comprising said immune checkpoint inhibitor, such as pembrolizumab.

In certain aspects, said pharmaceutical composition comprises petosemtamab and may be contained in a holder that is separate, meaning not physically linked, from a holder that contains said pharmaceutical composition comprising said immune checkpoint inhibitor, such as pembrolizumab.

Pharmaceutical compositions suitable for administration to human patients are typically formulated for parenteral administration, e.g., in a liquid carrier, or suitable for reconstitution into liquid solution or suspension for intravenous administration. The compositions may be formulated in dosage unit form for ease of administration and uniformity of dosage. Also included are solid preparations which are intended for conversion, shortly before use, to liquid preparations for either oral or parenteral administration. Such liquid forms include solutions, suspensions and emulsions.

### Instructions for Use of an EGFR Binding Antibody and an Immune Checkpoint Inhibitor of the Present Disclosure.

In certain aspects, nivolumab is administered at 240 mg every 2 weeks or 480 mg every 4 weeks, or 1 mg/kg followed by ipilimumab 3 mg/kg on the same day every 3 weeks for 4 doses, then 240 mg every 2 weeks or 480 mg every 4 weeks. In certain aspects, nivolumab is administered at 3 mg/kg every 2 weeks with ipilimumab 1 mg/kg every 6 weeks or at 360 mg every 3 weeks with ipilimumab 1 mg/kg every 6 weeks and 2 cycles of platinum-doublet chemotherapy. In certain aspects, Nivolumab is administered at 360 mg every 3 weeks with ipilimumab 1 mg/kg every 6 weeks. In certain aspects, nivolumab is administered at 240 mg every 2 weeks or 480 mg every 4 weeks, or at 3 mg/kg every 2 weeks, or at 3 mg/kg followed by ipilimumab 1 mg/kg on the same day every 3 weeks for 4 doses, then 240 mg every 2 weeks or 480 mg every 4 weeks.

Ipilimumab (sold under the brand name Yervoy™), is a monoclonal antibody and immune checkpoint inhibitor that works to activate the immune system by targeting CTLA-4, a protein receptor that downregulates the immune system. Ipilimumab was approved by the US Food and Drug Administration (FDA) in March 2011, for the treatment of melanoma.

In certain aspects, the immune checkpoint inhibitor is nivolumab and administered in an amount of 3 mg/kg as an intravenous infusion over (e.g. over 60 minutes) every 2 weeks until disease progression or unacceptable toxicity.

In certain aspects, nivolumab is used for head and neck cancer, such as recurrent or metastatic squamous cell carcinoma, and administered at 240 mg every 2 weeks or 480 mg every 4 weeks.

In certain aspects, the dosage forms and strength of nivolumab is an injection of 40 mg/4 mL (i.e. 10 mg/mL), 100 mg/10 mL (i.e. 10 mg/mL), 120 mg/12 mL (i.e. 10 mg/mL), or 240 mg/24 mL (i.e. 10 mg/mL) of a solution in a single-dose vial. In certain aspects, Nivolumab administration is by a 30-minute intravenous infusion.

In certain aspects, the immune checkpoint inhibitor is cemiplimab and is administered in an amount of 350 mg as an intravenous infusion (e.g. over 30 minutes) every 3 weeks, typically until disease progression or unacceptable toxicity. In certain aspects, administration is by intravenous infusion over 30 minutes through an intravenous line containing a sterile, in-line or add-on 0.2-micron to 5-micron filter. In certain aspects, the dosage forms and strength of cemiplimab is an injection of 350 mg/7 mL (i.e. 50 mg/mL) solution in a single-dose vial.

In certain aspects, the immune checkpoint inhibitor is dostarlimab (or Jemperli) and is administered as a first dose through a fourth dose of 500 mg every 3 weeks, followed by subsequent dosing beginning 3 weeks after dose 4 (dose 5 onwards): 1,000 mg every 6 weeks, as an intravenous infusion over (e.g. over 30 minutes) until disease progression or unacceptable toxicity. In certain aspects, the dosage forms and strength of dostarlimab is an injection of 500 mg/10 mL (i.e. 50 mg/mL) solution in a single-dose vial. In certain aspects, administer is by intravenous infusion over 30 minutes through an intravenous line using tubing made of polyvinyl chloride or platinum cured silicon; fittings made of polyvinyl chloride or polycarbonate; and a sterile, non-pyrogenic, low-protein binding, 0.2-micron, in-line or add-on filter.

In certain aspects, the immune checkpoint inhibitor is atezolizumab and is administered in an amount 840 mg every 2 weeks, 1200 mg every 3 weeks, or 1680 mg every 4 weeks as an intravenous infusion (e.g. over 60 minutes), typically until disease progression or unacceptable toxicity. If the first infusion is tolerated, all subsequent infusions are delivered over 30 minutes. In certain aspects, injection is an 840 mg/14 mL (i.e. 60 mg/mL) or 1200 mg/20 mL (i.e. 60 mg/mL) solution in a single-dose vial.

In certain aspects, the immune checkpoint inhibitor is avelumab and is administered in an amount of 10 mg/kg as an intravenous infusion (e.g. over 60 minutes) every 2 weeks, typically until disease progression or unacceptable toxicity. In certain aspects, administration is in an amount of 800 mg every 2 weeks as an intravenous infusion (e.g. over 60 minutes), typically until disease progression or unacceptable toxicity. In certain aspects, injection is of a 200 mg/10 mL (i.e. 20 mg/mL) solution in single-dose vial.

In certain aspects, the immune checkpoint inhibitor is avelumab and used at 800 mg every two weeks (800 mg q2w), optionally in combination with axitinib 5 mg orally twice daily. Avelumab is administered as an intravenous infusion over 60 minutes. The dosage form and strength is an injection of 200 mg/10 mL (20 mg/mL) solution in a single-dose vial.

In certain aspects, the immune checkpoint inhibitor is durvalumab and is administered in an amount of 10 mg/kg administered as an intravenous infusion (e.g. over 60 minutes) every 2 weeks until disease progression or unacceptable toxicity. In certain aspects, injection is of a 500 mg/10 mL (i.e. 50 mg/mL) or 120 mg/2.4 mL (i.e. 50 mg/mL) solution in a single-dose vial.

In certain aspects, the immune checkpoint inhibitor is pembrolizumab and administered in an amount of 200 mg as an intravenous infusion every 3 weeks. In certain aspects, said administration is a flat dose of 200 mg and occurs every 3 weeks until disease progression or unacceptable toxicity.

In certain aspects, the immune checkpoint inhibitor is pembrolizumab and administered in an amount of 400 mg as an intravenous infusion every 6 weeks. In certain aspects, said administration is a flat dose of 400 mg and occurs every 6 weeks until disease progression or unacceptable toxicity.

In certain aspects, the immune checkpoint inhibitor is pembrolizumab and is administered at 2 mg/kg (up to 200 mg) as an intravenous infusion every 3 weeks for pediatrics.

In certain aspects, the dosage form and strength of pembrolizumab injections is of 100 mg/4 mL (or 25 mg/mL) solution in a single-dose vial. In certain aspects, pembrolizumab is used as a single agent and is indicated for the first-line treatment of subjects with metastatic or with unresectable, recurrent HNSCC whose tumors express PD-L1 [Combined Positive Score (CPS)≥1] as determined by an FDA-approved test. In certain aspects, pembrolizumab administration is by a 30-minute intravenous infusion.

In certain aspects, the immune checkpoint inhibitor is penpulimab and its instructions for use include an administration of 200 mg once every two weeks (200 mg q2w). Administration typically is an intravenous injection.

In certain aspects, the immune checkpoint inhibitor is sintilimab and its instructions for use include an administration of 200 mg once every three weeks (q3w). Administration typically is an intravenous injection.

In certain aspects, the immune checkpoint inhibitor is tislelizumab and its instructions for use include an administration of 200 mg once every three weeks (200 mg q3w). Administration typically is an intravenous injection.

In certain aspects, the immune checkpoint inhibitor is retifanlimab and its instructions for use include an administration of 500 mg every four weeks (500 mg q4w). Administration typically is an intravenous injection.

In certain aspects, the immune checkpoint inhibitor is toripalimab for which recommended phase II dose was determined to be 3 mg/kg Q2W. The instructions for use include an administration of 3 mg/kg every two weeks. Administration typically is an intravenous injection.

Also, in certain aspects, the present disclosure comprises instructions for use of said antibody or functional part, derivative and/or analogue thereof that binds EGFR and optionally LGR5, such as petosemtamab. These instructions for use are provided in more detail in the present disclosure and relate to indications for usage (such as for the treatment of head and neck cancer), route of administration (such as intravenous injection), dosage amount (such as an amount of 1500 mg), a dosage irrespective of body weight of the subject (i.e. a flat dose) and the dosage interval (such as once every two weeks).

In certain aspects, said antibody or functional part, derivative and/or analogue thereof comprises petosemtamab (cf. Recommended INN list 83, WHO Drug Information Vol. 34, No. 1, 2020) and instructions for use include administration in an amount of 1500 mg as an intravenous infusion once every two weeks. In certain aspects, said dose is a flat dose of 1500 mg.

In certain aspects, said antibody or functional part, derivative and/or analogue thereof comprises petosemtamab and instructions for use include administration in an amount of 1100 mg as an intravenous infusion once every two weeks. In certain aspects, said dose is a flat dose of 1100 mg.

In certain aspects, petosemtamab and pembrolizumab are sequentially administered. In certain aspects, when pembrolizumab and petosemtamab are administered on the same day, pembrolizumab is administered after petosemtamab.

In certain aspects, petosemtamab is administered in an amount of 1500 mg once every two weeks and pembrolizumab is administered in an amount of 400 mg once every six weeks. In certain aspects, both said dosages are a flat dose.

In certain aspects, petosemtamab is administered in an amount of 1100 mg once every two weeks and pembrolizumab is administered in an amount of 400 mg once every six weeks. In certain aspects, both said dosages are a flat dose.

### Administration, Dosage Regime, Treatment

The therapeutic agent disclosed (e.g. petosemtamab) can be administered according to a suitable dosage, and suitable route (e.g., intravenous, intraperitoneal, intramuscular, intrathecal or subcutaneous). For example, a single bolus may be administered, several divided doses may be administered over time or the dose may be proportionally reduced or increased as indicated by the exigencies of the therapeutic situation. In certain aspects, a subject is administered a single dose of the antibody or functional part, derivative and/or analogue thereof as disclosed herein. In certain aspects, the therapeutic agent will be administered repeatedly, over a course of treatment. For example, in certain aspects, multiple (e.g., 2, 3, 4, 5, 6, 7, 8, 9, 10 or more) doses of the therapeutic agent are administered to a subject in need of treatment. In certain aspects, administrations of the therapeutic agent may be done weekly, biweekly or monthly.

A clinician may utilize preferred dosages as warranted by the condition of the patient being treated. The dose may depend upon a number of factors, including stage of disease, etc. Determining the specific dose that should be administered based upon the presence of one or more of such factors is within the skill of the artisan. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

In certain aspects, the therapeutic agent is administered at a dose of 0.1, 0.3, 1, 2, 3, 4, 5, 6, 7, 8, 9, or 10 mg/kg body weight. Alternatively, the therapeutic agent is administered at a dose of 0.5, 1, 2, 3, 4, 5, 6, 7, 8, 9, or 10 mg/kg body weight. In certain aspects, the therapeutic agent (e.g. petosemtamab) is provided to a subject using a flat dosage of 1500 mg. A flat dosage offers several advantages over body-surface or weight dosing as it reduces preparation time and reduces potential dose calculation mistakes. In certain aspects, the therapeutic agent is provided at a dosage of at least 500 mg. In certain aspects, said dosage is between 1100 to 2000 mg. In certain aspects, said dosage is between 1100 to 1800 mg. As is understood by the skilled person, the dosage can be administered over time. For example, the dosage may be administered by IV, for example with a 1-6 hour infusion, preferably a 2-4 hour infusion. In certain aspects, the therapeutic agent is administered once every 2 weeks. In particular, the flat dosages disclosed herein are suitable for use in adults and/or in subjects weighing at least 35 kg. In certain aspects, the subject is afflicted with head and neck cancer.

In certain aspects, said antibody or functional part, derivative and/or analogue thereof comprises petosemtamab and administrated in an amount of 1100 mg as an intravenous infusion once every two weeks. In certain aspects, said dose is a flat dose of 1100 mg.

In certain aspects, said antibody or functional part, derivative and/or analogue thereof comprises petosemtamab and is administrated in an amount which achieves human receptor target engagement for both EGFR and LGR5 of at least 90%, at least 95%, at least 99% across relevant body weights for a statistically significant number of subjects. Said amount of 90% may be achieved using a flat dose of about 1000 mg Q2W. Said amount of 95% may be achieved using a flat dose of about 1100 to about 1200 mg Q2W.

In certain aspects, a premedication regimen may be used. Such a regimen may be useful to reduce the likelihood or severity of an infusion-related reaction. Generally, a steroid such as dexamethasone and/or an antihistamine such as dexchlorpheniramine, diphenhydramine, or chlorpheniramine is administered (e.g., orally, intravenously) prior to treatment with a therapeutic agent mentioned herein.

The treatment method described herein is typically continued for as long as the clinician overseeing the patient's care deems the treatment method to be effective, i.e., that the patient is responding to treatment. Non-limiting parameters that indicate the treatment method is effective may include one or more of the following: decrease in tumor cells; inhibition of tumor cell proliferation; tumor cell elimination; progression-free survival; appropriate response by a suitable tumor marker (if applicable).

With regard to the frequency of administering the therapeutic agent, one of ordinary skill in the art will be able to determine an appropriate frequency. For example, a clinician can decide to administer the therapeutic agent relatively infrequently (e.g., once every two weeks) and progressively shorten the period between doses as tolerated by the patient. Exemplary lengths of time associated with the course of therapy in accordance with the claimed method include: about one week; two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years; about five years; perpetual (e.g., ongoing maintenance therapy). The foregoing duration may be associated with one or multiple rounds/cycles of treatment.

The efficacy of the treatment methods provided herein can be assessed using any suitable means. In certain aspects, the clinical efficacy of the treatment is analyzed using cancer cell number reduction as an objective response criterion. Patients, e.g., humans, treated according to the methods disclosed herein preferably experience improvement in at least one sign of cancer. In certain aspects, one or more of the following can occur: the number of cancer cells can be reduced; cancer recurrence is prevented or delayed; one or more of the symptoms associated with cancer can be relieved to some extent. In addition, in vitro assays to determine the T cell mediated target cell lysis. In certain aspects, tumor assessment is based on CT-scan and/or MRI scans, see, e.g., the RECIST 1.1 guidelines (Response Evaluation Criteria in Solid Tumours) (Eisenhauer et al., 2009 Eur J Cancer 45:228-247). Such assessments generally take place every 4-8 weeks after treatment.

In certain aspects, the tumor cells are no longer detectable following treatment as described herein. In certain aspects, a subject is in partial or full remission. In certain aspects, a subject has an increased overall survival, median survival rate, and/or progression free survival.

The therapeutic agent (i.e., an antibody or functional part, derivative and/or analogue thereof that comprises a variable domain that binds an extracellular part of EGFR and a variable domain that binds an extracellular part of LGR5) is used herein in conjunction with an immune checkpoint inhibitor as a further therapeutic agent for their particular usefulness against the cancer that is being treated.

In certain aspects, at the start of treatment, at least one, more than one or all of the following inclusion factors IF1-IF16 are applicable to subjects for treatment. In certain aspects, the subject comprises or complies with all of IF1-IF16:
IF 1. Having an age of at least 18.
IF 2. Having histologically or cytologically confirmed solid tumors with evidence of metastatic or locally advanced disease not amenable to standard therapy with curative intent for subjects with locally advanced unresectable or metastatic disease for HNSCC with tumors expressing PD-L1, CPS≥1, as determined by an FDA-approved test in the US, or by an approved equivalent test in other countries.
IF 3. Not having received previous systemic therapy administered in the recurrent or metastatic setting, although previous systemic therapy as part of multimodal treatment for locally advance disease is allowed if ended at least 6 months prior to being administered petosemtamab and/or pembrolizumab.
IF 4. Having a HNSCC primary tumor locations in the oropharynx, oral cavity, hypopharynx or larynx.
IF 5. Not having received previous anti-cancer treatment with an anti-PD-L1, anti-PD-1 or anti-EGFR therapy.
IF 6. Having a baseline tumor sample (e.g. a formalin-fixed paraffin-embedded block, [FFPE]) from a metastatic or primary site. If the subject has such an available tumor sample from sample collection with sufficient material (at least 20 slides with >20% tumor content) and has not received further anticancer treatment since said sample collection, a new tumor biopsy at baseline is not necessary. Archival FFPE slides are not acceptable. Primary tumor material is only acceptable if the patient has not been treated with anti-EGFR.
IF 7. Being amenable for a biopsy.
IF 8. Having a measurable disease as defined by RECIST version 1.1 by radiologic methods.
IF 9. Having an Eastern Cooperative Oncology Group (ECOG) performance status of 0 or 1.
IF 10. Life expectancy ≥12 weeks, typically assessed as per investigator.
IF 11. Left ventricular ejection fraction (LVEF) at least 50% by echocardiogram (ECHO) or multigated acquisition scan (MUGA).
IF 12. Adequate organ function:
   IF 12.1 Having an Absolute Neutrophil Count (ANC) of at least 1.5×10⁹/L.
   IF 12.2 Having a hemoglobin level of at least 9 g/dL.
   IF 12.3 Having a platelets level of at least 100×10⁹/L.
   IF 12.4 Having a corrected total serum calcium within normal ranges.
   IF 12.5 Having a serum magnesium level within normal ranges or corrected with supplements.
   IF 12.6 Having an Alanine aminotransferase (ALT), Aspartate aminotransferase (AST) of equal to or less than 2.5 times the upper limit of normal (ULN) and total bilirubin of equal to or less than 1.5 times ULN, with the proviso that in case subjects have Gilbert's syndrome, total bilirubin is less than or equal to 3.0 times ULN or direct bilirubin is less than or equal to 1.5 times ULN; with the proviso that in case of liver involvement, ALT/AST is equal to or less than 5 times ULN and total bilirubin is equal to or less than 2 times ULN, or in case of hepatocellular carcinoma (HCC) with a Child-Pugh class A score, total bilirubin is less than 3 mg/dL.
   IF 12.7 Having a serum creatinine level of less than 1.5×ULN or having a creatinine clearance of at least 60 mL/min calculated according to the Cockroft and Gault formula or Modification of Diet in Renal Disease (MDRD) formula for patients aged 65 years or more IF 12.8 Having a serum albumin level of at least 3 g/dL.
   IF 12.9 Having am International Normalized Ratio (INR) or Prothrombin Time (PT) level of less than 1.5×ULN, unless patient receives anticoagulant therapy and in therapeutic range of intended used anticoagulant.
   IF 12.10 Having an Activated Partial Thromboplastin Time (APTT) or PTT less than 1.5×ULN, unless patient is receiving anticoagulant therapy and is in therapeutic range of intended used anticoagulant.
   IF 13.1. Having a negative test result for a human immunodeficiency virus infection with date of testing which does not lie more than six months prior to treatment according to the present disclosure.
   IF 13.2 If having a positive test result for a human immunodeficiency virus infection, provided the CD4+ count is at least 300/μL, viral load is undetectable and the subject is being treated with Highly Active Anti-Retroviral Therapy (HAART).
IF 14. Having an active hepatitis B (HBsAg positive) infection but receive antiviral treatment with lamivudine, tenofovir, entecavir, or other antiviral agents, starting at least seven days start of treatment according to the present disclosure.
IF 15. Having an antecedent of hepatitis B (being anti-HBc positive, HBsAg and hepatitis B virus [HBV]-DNA negative).
IF 16. Having a positive test for hepatitis C virus (HCV) ribonucleic acid (RNA) and which subjects wherein said HCV infection resolved spontaneously (i.e. having positive HCV antibodies without detectable HCV-RNA) or subjects who achieved a sustained response after antiviral treatment and show absence of detectable HCV RNA at least six months with the use of IFN-free regimens or at least 12 months with the use of IFN-based regimens, after cessation of antiviral treatment.

In certain aspects, all values for organ function measurements according to IF12 have an upper limit observed with healthy subjects.

In certain aspects, the subject for treatment complies with any one or more factors selected from the group consisting of IF1-IF16. In certain aspects, the subject for treatment complies with factors IF2, IF3, IF4, IF5, IF8, IF9, IF10, IF11, IF12, IF13, IF14, IF15 and IF16. In certain aspects, the subject for treatment complies with factors IF2, IF3, IF4 and IF5. In certain aspects, the subject for treatment complies with factor IF3.

In certain aspects, at the start of treatment, at least one, more than one or all of the following exclusion factors EF1-EF are applicable to subjects for treatment:
EF 1. Having central nervous system metastases that are untreated or symptomatic, or require radiation, surgery, or continued steroid therapy to control symptoms within 14 days from start of treatment according to the present disclosure.
EF 2. Having leptomeningeal involvement.
EF 3. Participation in a further clinical trial or treatment with any investigational drug within four weeks prior to start of treatment according to the present disclosure.
EF 4. Having any systemic anticancer therapy within four weeks or five half-lives, whichever is longer, of the first dose according to the present disclosure. For cytotoxic agents that have major delayed toxicity (e.g. mitomycin C, nitrosoureas), or anticancer immunotherapies, a washout period of six weeks prior to prior to start of treatment according to the present disclosure is required.
EF 5. Having a requirement for immunosuppressive medication (eg, methotrexate, cyclophosphamide).
EF 6. Having major surgery or radiotherapy within three weeks of start of treatment according to the present disclosure. Patients who received prior radiotherapy to at least 25% or more of bone marrow are excluded, irrespective of when it was received.
EF 7. Having persistent Grade >1 clinically significant toxicities related to prior antineoplastic therapies (except for alopecia); with the proviso that stable sensory neuropathy of Grade 2 (or less) National Cancer Institute-Common Terminology Criteria for Adverse Events (NCI-CTCAE) v4.03 or v5.0 is allowed or as current at the time of administration.
EF 8. Having a history of hypersensitivity reaction or any toxicity attributed to human proteins or any of the excipients that warranted permanent cessation of these agents.
EF 9. Exhibiting uncontrolled hypertension (systolic BP>150 mmHg and/or diastolic BP>100 mmHg) with appropriate treatment, or unstable angina.
EF 10. Having a history of congestive heart failure of Class II-IV New York Heart Association (NYHA) criteria, or serious cardiac arrhythmia requiring treatment (except atrial fibrillation, paroxysmal supraventricular tachycardia).
EF 11. Having had a myocardial infarction within six months from start of treatment according to the present disclosure.
EF 12. Having a history of prior malignancies, with the exception of excised cervical intraepithelial neoplasia or nonmelanoma skin cancer, or curatively treated cancer deemed at low risk for recurrence with no evidence of disease for at least three years prior to start of treatment according to the present disclosure.
EF 13. Having dyspnea at rest of any origin, or other diseases requiring continuous oxygen therapy.
EF 14. Having a history of interstitial lung disease (ILD) (eg, pneumonitis or pulmonary fibrosis), or evidence of ILD on baseline chest computerized tomography (CT) scan.
EF 15. Having a serious illness or medical conditions including, but not limited to, uncontrolled active infection, clinically significant pulmonary, metabolic or psychiatric disorders at start of treatment according to the present disclosure.
EF 16. Subjects having an active hepatitis B surface antigen infection (HBsAg positive) without receiving antiviral treatment.
EF 17. Subjects having cirrhotic status of Child-Pugh class B or C; subjects having fibrolamellar HCC, subjects having sarcomatoid HCC, or subjects having mixed cholangiocarcinoma and HCC.
EF 18. Pregnant subjects or being a breastfeeding subject; subjects of childbearing potential must use highly effective contraception methods prior to start of treatment according to the present disclosure, for the duration of said treatment, and for six months after the last dose of petosemtamab administration.
EF 19. Having a diagnosis of immunodeficiency, or is receiving systemic steroid therapy or any form of immunosuppressive therapy within seven days prior to start of treatment according to the present disclosure. Of note, corticosteroid use as premedication for allergic reactions or infusion-related reactions specified herein are allowed.
EF 20. Having an active autoimmune disease that has required systemic immune suppressive treatment in the two years prior to start of treatment according to the present disclosure; with the proviso that receiving replacement therapy (eg, thyroxine, insulin, or physiologic corticosteroid replacement therapy for adrenal or pituitary insufficiency, etc.) is not considered immune suppressive treatment.
EF 21. Having had an allogeneic tissue/solid organ transplant prior to start of treatment according to the present disclosure.
EF 22. Having a primary tumor site of the nasopharynx of any histology.

In certain aspects, the subject for treatment complies with any one or more factors selected from the group consisting of EF1-EF22. In certain aspects, the subject for treatment complies with all of the factors EF1-EF22. In certain aspects, the subject for treatment complies with factors IF13, IF16, IF19, IF20 and IF21.

ECOG Performance Status Scoring Grade Definition is as followed in the art, meaning: 0 Fully active, able to carry on all pre-disease performance without restriction. 1 Restricted in physically strenuous activity but ambulatory and able to carry out work of a light or sedentary nature, e.g, light housework, office work. 2 Ambulatory and capable of all self-care but unable to carry out any work activities. Up and about more than 50% of waking hours. 3 Capable of only limited self-care, confined to bed or chair more than 50% of waking hours. 4 Completely disabled. Cannot carry on any self-care. Totally confined to bed or chair. 5 Dead.

The Child-Pugh scoring, also referred to as the Child-Pugh classification, the Child-Turcotte-Pugh (CTP) calculator or the Child Criteria, herein is applied in accordance with standard clinical practice. The Child-Pugh score is determined by scoring five clinical measures of liver disease and the possibility of eventual liver failure. A score of 1, 2, or 3 is given to each measure, with 3 being the most severe. The five clinical measures are total bilirubin, serum albumin level, prothrombin time (or prolongation or INR as a time for blood to clot), ascites, and hepatic encephalopathy. Class A means 5 to 6 points, least severe liver disease and a one- to five-year survival rate of 95 percent. Class B: 7 to 9 points, moderately severe liver disease and a one- to five-year survival rate of 75 percent. Class C: 10 to 15 points, most severe liver disease and a one- to five-year survival rate of 50 percent. Per clinical measure, the following points are given. Encephalopathy: None=1 p, Grade 1 and 2=2 p, Grade 3 and 4=3 pts. Ascites: none=1 pt, slight=2 pts, moderate=3 pts. Bilirubin: under 2 mg/ml=1 pt, 2 to 3 mg/ml=2 pts, over 3 mg/ml=3 pts. Albumin: greater than 3.5 mg/ml=1 pt, 2.8 to 3.5 mg/ml=2 pts, less than 2.8 mg/ml=3 pts. Prothrombin Time (PT, seconds prolonged): less than 4 sec=1 pt, 4 to 6 sec=2 pts, over 6 sec=3 pts. Alternatively, the International Normalized Ratio (INR) will be used as a substitute for PT, with INR under 1.7=1 pt, INR 1.7 to 2.2=2 pts, INR above 2.2=3 pts.

Administration of the therapeutic agent of the present disclosure may be premedicated, meaning medication is administered to the subject prior to being administered the antibody or immune checkpoint of the present invention. In certain aspects, a dose of 1500 mg petosemtamab is premedicated with an antihistamine, pain reducing medication, fever reducing medication and/or anti-inflammatory medication.

Key permitted medications are as follows. In certain aspects, treatment of the present disclosure comprises premedication with paracetamol/acetaminophen, antihistamines, or corticosteroids. In certain aspects, said premedication is administered in the event of infusion-related reactions, hypersensitivity and/or allergic reactions, according to standard local clinical practice.

In certain aspects, treatment of the present disclosure further comprises all medication necessary for the patient's safety and well-being, and which is not expected to interfere with evaluation of the study drug, may be given at the investigator's discretion. Also permitted is supportive treatment of symptoms and adverse events, or standard treatment of concomitant conditions, including aspirin, transfusion support, granulocyte colony-stimulating factor, antibiotics, inhaled steroids (for asthma), antiemetics, antidiarrheals (e.g., loperamide), and bisphosphonates (according to their product license and routine clinical practice). Also permitted are adjuvant hormone therapy agents for curatively treated cancer deemed at low risk for recurrence with no evidence of disease are allowed. This can include adjuvant Luteinizing Hormone Releasing Hormone (LHRH) agonists for early stage breast cancer in combination with an antiestrogen (for 5 years), and also LHRH agonists+/−antiandrogen for localized prostate cancer. Also permitted is concurrent radiation treatment during said treatment for symptom control without evidence of progression.

Key prohibited medications are as follows. In certain aspects, treatment of the present disclosure does not comprise concomitant medications for risk of immunosuppression. Such medication typically includes chronic oral corticosteroids (>10 mg/day prednisone equivalent, excluding inhaled and topical steroids), Tumor Necrosis Factor (TNF)-alpha inhibitors, anti-T cell antibodies, other immunosuppressive medication. In certain aspects, treatment of the present disclosure does not comprise any investigational drug or other anticancer therapy during said treatment or within four weeks, or, if known, five half-lives, whichever is longer, prior to administration of the first dose of the present treatment. In certain aspects, treatment of the present disclosure does not comprise cytotoxic agents that have major delayed toxicity (e.g. mitomycin C, nitrosoureas, or anticancer immunotherapies) for which a washout period of six weeks is required. In certain aspects, treatment of the present disclosure does not comprise herbal remedies for cancer treatment that have not commenced prior to first administration of a therapeutic agent of the present disclosure. Herbal remedies for cancer treatment that continue during said treatment are permitted.

In certain aspects, treatment of the present disclosure does not comprise major surgery or radiotherapy within three weeks prior to administration of the first dose of said treatment, or prior radiotherapy to at least 25% of total bone marrow of the subject to be treated.

During said treatment, concomitant medication with chronic oral corticosteroids (such as >10 mg/day prednisone equivalent, excluding inhaled and topical steroids), Tumor Necrosis Factor (TNF)-alpha inhibitors, anti-T cell antibodies or other immunosuppressive medication is not allowed. Hence, in certain aspects, there is no concomitant medication with chronic oral corticosteroids (such as >10 mg/day prednisone equivalent, excluding inhaled and topical steroids), Tumor Necrosis Factor (TNF)-alpha inhibitors, anti-T cell antibodies or other immunosuppressive medication during treatment according to the present disclosure.

The compounds and compositions disclosed herein are useful as therapy and in therapeutic treatments and may thus be useful as medicaments and used in a method of preparing a medicament.

### Kit-of-Parts, and Combinations of Therapeutic Agents

In certain aspects, the present disclosure also provides a kit of parts comprising an antibody or functional part, derivative and/or analogue thereof as defined herein, an immune checkpoint inhibitor as defined herein and instructions for use of said antibody or functional part, derivative and/or analogue thereof and for use of said immune checkpoint inhibitor.

In certain aspects, the present disclosure also provides a kit of parts comprising an antibody or functional part, derivative and/or analogue thereof as defined herein, instructions for use of said antibody or functional part, derivative and/or analogue thereof and instructions for use of an immune checkpoint inhibitor as defined herein.

Also, the present disclosure provides a kit of parts comprising an antibody or functional part, derivative and/or analogue thereof that comprises a variable domain that can bind an extracellular part of EGFR and optionally LGR5, an immune checkpoint inhibitor and instructions for use of said antibody or functional part, derivative and/or analogue thereof and for use of said immune checkpoint inhibitor.

Also, the present disclosure provides a combination of an antibody or functional part, derivative and/or analogue thereof that comprises a variable domain that can bind an extracellular part of EGFR and optionally LGR5 and an immune checkpoint inhibitor as mentioned herein for use in the treatment of cancer in a subject in need thereof.

Also, the present disclosure provides a combination of an immune checkpoint inhibitor, as mentioned herein, instructions for use of said immune checkpoint inhibitor in the treatment of cancer in a subject, as well as instructions for use in the treatment of said cancer in a subject of an antibody or functional part, derivative and/or analogue thereof as mentioned herein that comprises a variable domain that can bind an extracellular part of EGFR and optionally LGR5.

Also, the present disclosure provides a combination of an antibody or functional part, derivative and/or analogue thereof that comprises a variable domain that can bind an extracellular part of EGFR and optionally LGR5, as mentioned herein, instructions for use of said antibody or functional part, derivative and/or analogue thereof in the treatment of cancer in a subject, as well as instructions for use in the treatment of said cancer in a subject of an immune checkpoint inhibitor as mentioned herein.

In certain aspects, the present disclosure provides a pharmaceutical composition comprising an antibody or functional part, derivative and/or analogue thereof that comprises a variable domain that can bind an extracellular part of EGFR and optionally LGR5 of the present disclosure and instructions for use thereof with an immune checkpoint inhibitor in the treatment of said cancer.

In certain aspects, the present disclosure provides a pharmaceutical composition for the treatment of a cancer, comprising an antibody or functional part, derivative and/or analogue thereof that comprises a variable domain that can bind an extracellular part of EGFR of the present disclosure and optionally LGR5 and a pharmaceutical composition for the treatment of said cancer, comprising an immune checkpoint inhibitor of the present disclosure.

In certain aspects, the present disclosure provides a pharmaceutical composition for use in the treatment of cancer comprising an antibody or functional part, derivative and/or analogue thereof that comprises a variable domain that can bind an extracellular part of EGFR of the present disclosure and optionally LGR5, wherein the pharmaceutical composition is administered in combination with an immune checkpoint inhibitor of the present disclosure.

In certain aspects, the present disclosure relates to a pharmaceutical composition for the treatment of a cancer comprising an antibody or functional part, derivative and/or analogue thereof that comprises a variable domain that can bind an extracellular part of EGFR of the present disclosure and optionally LGR5, wherein a subject to be treated in the treatment is administered an immune checkpoint inhibitor prior to, simultaneously with, or after administration of said bispecific antibody.

In certain aspects, the present disclosure relates to a pharmaceutical composition for the treatment of a cancer in a subject comprising an immune checkpoint inhibitor, wherein said subject to be treated is administered an antibody or functional part, derivative and/or analogue thereof that comprises a variable domain that can bind an extracellular part of EGFR and optionally LGR5 of the present disclosure prior to, simultaneously with, or after administration of said immune checkpoint inhibitor.

The present disclosure thus relates to a combination of medicaments for the treatment of cancer in a subject which comprises administration to said subject of multiple, different medicaments for treating said cancer, which treatment comprises simultaneous, sequential or separate administration of said medicaments. In certain aspects, said medicament comprises an antibody or functional part, derivative and/or analogue thereof that comprises a variable domain that can bind an extracellular part of EGFR and optionally LGR5 of the present disclosure, and said other, different medicament comprises an immune checkpoint inhibitor.

In certain aspects, a kit-of-parts or a combination comprises instructions for dosing of petosemtamab at 1500 mg. In certain aspects, said kit comprise instructions for use of petosemtamab for dosing at 1500 mg once every two weeks. In certain aspects, said kit comprises instructions for use of petosemtamab and an immune checkpoint inhibitor, such as pembrolizumab, in the treatment of head and neck cancer. In certain aspects, said kit comprise said kit comprises instructions for use of petosemtamab and said immune checkpoint inhibitor in the treatment of head and neck squamous cell cancer. In certain aspects, said kit comprise said kit comprises instructions for use of petosemtamab and pembrolizumab. In certain aspects, said kit comprises instructions for use of petosemtamab and pembrolizumab as mentioned herein, such a 400 mg, q6w for pembrolizumab and 1500 mg, q2w, of petosemtamab. In certain aspects, said kit comprises instructions for flat dosing of petosemtamab and flat dosing of pembrolizumab.

In certain aspects, the present disclosure also provides a combination of an antibody or functional part, derivative and/or analogue thereof as defined herein and an immune checkpoint inhibitor as defined herein for use in the treatment of a cancer in a subject in need thereof.

In certain aspects, the present disclosure also provides an immune checkpoint inhibitor, as defined herein for the treatment of a cancer in a subject in need thereof, wherein the immune checkpoint inhibitor is for simultaneous or sequential administration with an antibody or functional part, derivative and/or analogue thereof as defined herein.

In certain aspects, said antibody or functional part, derivative and/or analogue thereof is administered to a subject having cancer, to which said immune checkpoint inhibitor has been administered or will be administered. In certain aspects, said immune checkpoint inhibitor is administered to a subject having cancer, to which said antibody or functional part, derivative and/or analogue thereof has been administered or will be administered.

In certain aspects, the present disclosure also provides a combination of an antibody or functional part, derivative and/or analogue thereof, as defined herein, instructions for use in the treatment of a cancer in a subject of said antibody or functional part, derivative and/or analogue thereof, and instructions for use in the treatment of a cancer in a subject of an immune checkpoint inhibitor as defined herein.

In certain aspects, the present disclosure also provides a combination of an immune checkpoint inhibitor, as defined herein, instructions for use of said inhibitor in the treatment of a cancer in a subject and instructions for use of an antibody or functional part, derivative and/or analogue thereof as defined herein in the treatment of said cancer.

In certain aspects, the instructions for use comprise the amount of the immune checkpoint inhibitor and the amount of the antibody or functional part, derivative and/or analogue thereof to be used, the dosing interval and cancer to be treated.

All documents and references, including Genbank entries, patents and published patent applications, and websites, described herein are each expressly incorporated herein by reference to the same extent as if were written in this document in full or in part.

For the purpose of clarity and a concise description features are described herein as part of the same or separate parts of the disclosure, however, it will be appreciated that the scope of the invention may include preferred aspects having combinations of all or some of the features described.

The invention is now described by reference to the following examples, which are illustrative only, and are not intended to limit the present invention. While the invention has been described in detail and with reference to specific aspects thereof, it will be apparent to one of skill in the art that various changes and modifications can be made thereto without departing from the spirit and scope thereof.

### List of Clauses

### Clauses

1. An antibody or functional part, derivative and/or analogue thereof that comprises a variable domain that binds an extracellular part of EGFR for use in the treatment of a cancer in a subject, wherein the treatment further comprises administering an immune checkpoint inhibitor.
2. Use of an antibody or functional part, derivative and/or analogue thereof that comprises a variable domain that binds an extracellular part of EGFR and an immune checkpoint inhibitor in the manufacture of one or more medicaments for treating a cancer in a subject.
3. The use of clause 2, wherein the antibody or functional part, derivative and/or analogue thereof and the immune checkpoint inhibitor are used to manufacture separate medicaments or medicament formulations.
4. The use of clause 2 or 3, wherein the treatment of cancer comprises administration of the antibody or functional part, derivative and/or analogue thereof and the immune checkpoint inhibitor.
5. A method of treating cancer in a subject, the method comprising administering an effective amount of an antibody or functional part, derivative and/or analogue thereof that comprises a variable domain that binds an extracellular part of EGFR and an effective amount of an immune checkpoint inhibitor to the subject.
6. The antibody or functional part, derivative and/or analogue thereof, or the use or the method of any one of the preceding clauses, wherein said immune checkpoint inhibitor comprises a PD-L1 inhibitor, PD-L2 or PD-1 inhibitor.
7. The antibody or functional part, derivative and/or analogue thereof, or the use or the method of any one of the preceding clauses, wherein said immune checkpoint inhibitor comprises a PD-L2 inhibitor.
8. The antibody or functional part, derivative and/or analogue thereof, or the use or the method of any one of the preceding clauses, wherein said immune checkpoint inhibitor comprises a PD-1 inhibitor.
9. The antibody or functional part, derivative and/or analogue thereof, or the use or the method of any one of the preceding clauses, wherein said immune checkpoint inhibitor comprises nivolumab, pembrolizumab, cemiplimab, penpulimab, retifanlimab, sintilimab, tislelizumab, toripalimab, dostarlimab, atezolizumab, avelumab or durvalumab, in particular pembrolizumab.
10. The antibody or functional part, derivative and/or analogue thereof, or the use or the method of any one of the preceding clauses, wherein said immune checkpoint inhibitor comprises or is pembrolizumab.
11. The antibody or functional part, derivative and/or analogue thereof, or the use or the method of any one of the preceding clauses, wherein the cancer is an adenocarcinoma or a squamous cell carcinoma or in particular a head and neck cancer.
12. The antibody or functional part, derivative and/or analogue thereof, or the use or the method of any one of the preceding clauses, wherein the cancer is a head and neck cancer.
13. The antibody or functional part, derivative and/or analogue thereof, or the use or the method of any one of the preceding clauses, wherein the cancer is squamous cell carcinoma of the head and neck.
14. The antibody or functional part, derivative and/or analogue thereof, or the use or the method of any one of the preceding clauses, wherein the cancer is a cancer of the pharynx (including nasopharynx, oropharynx and hypopharynx), oral cavity, larynx, paranasal sinuses, nasal cavity or salivary glands.
15. The antibody or functional part, derivative and/or analogue thereof, or the use or the method of any one of the preceding clauses, wherein the cancer is a cancer of the oropharynx, oral cavity, hypopharynx or larynx.
16. The antibody or functional part, derivative and/or analogue thereof, or the use or the method of any one of the preceding clauses, wherein the primary location of the cancer is in the oropharynx, oral cavity, hypopharynx or larynx.
17. The antibody or functional part, derivative and/or analogue thereof, or the use or the method of any one of the preceding clauses, which cancer expresses EGFR, and optionally LGR5.
18. The antibody or functional part, derivative and/or analogue thereof, or the use or the method of any one of the preceding clauses, which cancer expresses EGFR characterized by an IHC score of 2+ or 3+ or characterized by an H score for EGFR of more than 50, such as 80, 100 or 200, but not more than 300.
19. The antibody or functional part, derivative and/or analogue thereof, or the use or the method of any one of the preceding clauses, wherein the cancer expresses PD-L1.
20. The antibody or functional part, derivative and/or analogue thereof, or the use or the method of any one of the preceding clauses, wherein the cancer has a Combined Positive Score (CPS) for PD-L1 expression of 1 or more, but not more than 100.
21. The antibody or functional part, derivative and/or analogue thereof, or the use or the method of any one of the preceding clauses, wherein the cancer has a Combined Positive Score (CPS) for PD-L1 expression of between (and including) 1 or more and up to 20 (not including).
22. The antibody or functional part, derivative and/or analogue thereof, or the use or the method of any one of the preceding clauses, wherein the cancer has a Combined Positive Score (CPS) for PD-L1 expression of between (and including) 20 or more and up to 100 (including).
23. The antibody or functional part, derivative and/or analogue thereof, or the use or the method of any one of clauses 20-22, wherein CPS is determined using IHC.
24. The antibody or functional part, derivative and/or analogue thereof, or the use or the method of any one of clauses 20-23, wherein CPS is determined by IHC using clone 22C3.
25. The antibody or functional part, derivative and/or analogue thereof, or the use or the method of any one of the preceding clauses, wherein the subject or cancer is positive for p16 status.
26. The antibody or functional part, derivative and/or analogue thereof, or the use or the method of clause 25, wherein the cancer is, or the subject suffers from (primary) oropharyngeal cancer.
27. The antibody or functional part, derivative and/or analogue thereof, or the use or the method of any one of clauses 1-24, wherein the subject or cancer is negative for p16 status.
28. The antibody or functional part, derivative and/or analogue thereof, or the use or the method of clause 27, wherein the cancer is or subject suffers from a head and neck cancer other than (primary) oropharyngeal cancer.
29. The antibody or functional part, derivative and/or analogue thereof, or the use or the method of any of clauses 25-28, wherein p16 status is determined using IHC.
30. The antibody or functional part, derivative and/or analogue thereof, or the use or the method of any one of the preceding clauses, wherein the cancer expresses LGR5.
31. The antibody or functional part, derivative and/or analogue thereof, or the use or the method of any one of the preceding clauses, wherein the cancer is ISH positive for LGR5.
32. The antibody or functional part, derivative and/or analogue thereof, or the use or the method of clause 31, wherein LGR5 expression is determined using In-Situ Hybridization (ISH), RNA sequencing, Tissue MicroArray (TMA) staining or by calculating the H-score for LGR5.
33. The antibody or functional part, derivative and/or analogue thereof, or the use or the method of any one of the preceding clauses, wherein said subject has not received prior anti-cancer treatment for treatment of said cancer.
34. The antibody or functional part, derivative and/or analogue thereof, or the use or the method of any one of the preceding clauses, wherein said subject has not received prior anti-cancer treatment for treatment of said cancer at least six months prior to receiving said antibody or functional part, derivative and/or analogue thereof and said immune checkpoint inhibitor.
35. The antibody or functional part, derivative and/or analogue thereof, or the use or the method of clause 34, wherein said subject has received multimodal treatment, which comprises surgery, radiotherapy and/or platinum-containing chemotherapy, such as cisplatin, more than six months prior to receiving said antibody or functional part, derivative and/or analogue thereof and said immune checkpoint inhibitor.
36. The antibody or functional part, derivative and/or analogue thereof, or the use or the method of clause 35, wherein said subject underwent said multimodal treatment to treat curable local disease.
37. The antibody or functional part, derivative and/or analogue thereof, or the use or the method of clause 34, wherein said prior anti-cancer treatment comprises treatment with an anti PD-L1, anti PD-1 or anti-EGFR therapy.
38. The antibody or functional part, derivative and/or analogue thereof, or the use or the method of clause 34, wherein said prior anti-cancer treatment comprises treatment with chemotherapy, immune therapy, an anti-EGFR agent, an antibody targeting EGFR, cetuximab, a PD-1 inhibitor or a PD-L1 inhibitor.
39. The antibody or functional part, derivative and/or analogue thereof, or the use or the method of any one of the preceding clauses, wherein the subject is a mammal.
40. The antibody or functional part, derivative and/or analogue thereof, or the use or the method of any one of the preceding clauses, wherein the subject is a human subject.
41. The antibody or functional part, derivative and/or analogue thereof, or the use or the method of any one of the preceding clauses, wherein said treatment comprises a weekly, biweekly or triweekly administration of the antibody or functional part, derivative and/or analogue thereof, preferably biweekly, more preferably the subject is administered at least 3 or more biweekly dosages of the antibody or functional part, derivative and/or analogue thereof.
42. The antibody or functional part, derivative and/or analogue thereof, or the use or the method of any one of the preceding clauses, wherein said treatment comprises a biweekly administration of petosemtamab as the antibody to the subject.
43. The antibody or functional part, derivative and/or analogue thereof, or the use or the method of any one of the preceding clauses, wherein said treatment comprises a biweekly administration of a dose of 1100 or 1500 mg of petosemtamab to the subject.
44. The antibody or functional part, derivative and/or analogue thereof, or the use or the method of any one of the preceding clauses, wherein said treatment comprises administering a dose of 1100 or 1500 mg of petosemtamab to the subject.
45. The antibody or functional part, derivative and/or analogue thereof, or the use or the method of any one of the preceding clauses, wherein the antibody or functional part, derivative and/or analogue thereof is intravenously administered to the subject.
46. The antibody or functional part, derivative and/or analogue thereof, or the use or the method of any one of the preceding clauses, wherein said treatment comprises administration of a dose of 400 mg of pembrolizumab as immune check point inhibitor once every six weeks to the subject.
47. The antibody or functional part, derivative and/or analogue thereof, or the use or the method of any one of the preceding clauses, wherein administration of petosemtamab and pembrolizumab are sequentially administered.
48. The antibody or functional part, derivative and/or analogue thereof, or the use or the method of any one of the preceding clauses, wherein if pembrolizumab and petosemtamab are administered on the same day, pembrolizumab is administered after petosemtamab.
49. The antibody or functional part, derivative and/or analogue thereof, or the use or the method of any one of the preceding clauses, wherein during said treatment, concomitant medication with chronic oral corticosteroids (such as >10 mg/day prednisone equivalent, excluding inhaled and topical steroids), Tumor Necrosis Factor (TNF)-alpha inhibitors, anti-T cell antibodies or other immunosuppressive medication is not allowed.
50. The antibody or functional part, derivative and/or analogue thereof, or the use or the method of any one of the preceding clauses, wherein the antibody or functional part, derivative and/or analogue thereof is ADCC enhanced.
51. The antibody or functional part, derivative and/or analogue thereof, or the use or the method of any one of the preceding clauses, wherein the antibody or functional part, derivative and/or analogue thereof is afucosylated.
52. The antibody or functional part, derivative and/or analogue thereof, or the use or the method of any one of the preceding clauses, wherein the cancer is recurrent, unresectable, locally advanced and/or metastatic cancer.
53. The antibody or functional part, derivative and/or analogue thereof, or the use or the method of any one of the preceding clauses, the antibody or functional part, derivative and/or analogue thereof is multispecific.
54. The antibody or functional part, derivative and/or analogue thereof, or the use or the method of any one of the preceding clauses, wherein the antibody or functional part, derivative and/or analogue thereof is bispecific.
55. The antibody or functional part, derivative and/or analogue thereof, or the use or the method of any one of the preceding clauses, wherein the antibody comprises a variable domain that does not bind EGFR.
56. The antibody or functional part, derivative and/or analogue thereof, or the use or the method of any one of the preceding clauses, wherein the antibody comprises a variable domain that binds LGR5.
57. The antibody or functional part, derivative and/or analogue thereof, or the use or the method of any one of clauses 1-55, wherein the antibody is a monovalent antibody or wherein the antibody comprises said EGFR binding variable domain as the only variable domain.
58. The antibody or functional part, derivative and/or analogue thereof, or the use or the method of any one of the preceding clauses, wherein the treatment comprises or is preceded by a step of diagnosing or testing the subject for EGFR, LGR5 and/or PD-L1 expression.
59. The antibody or functional part, derivative and/or analogue thereof, or the method of any one of the preceding clauses, wherein the treatment comprises or is preceded by a step of diagnosing or testing the subject for PD-L1 expression.
60. The antibody or functional part, derivative and/or analogue thereof, or the method of clause 58 or 59, wherein diagnosing or testing is by ISH or IHC.
61. The antibody or functional part, derivative and/or analogue thereof, or the method of any one of clauses 58-60, wherein diagnosing or testing for PD-L1 expression is by IHC.
62. The antibody or functional part, derivative and/or analogue thereof, or the method of any one of clauses 58-60, wherein diagnosing or testing for PD-L1 expression is by establishing CPS for PD-L1.
63. The antibody or functional part, derivative and/or analogue thereof, or the method of clause 63, wherein diagnosing or testing for PD-L1 expression is by establishing CPS for PD-L1 for expression as determined by IHC.
64. The antibody or functional part, derivative and/or analogue thereof, or the method of clause 63, wherein CPS for PD-L1 expression is determined by IHC using clone 22C3.
65. The antibody or functional part, derivative and/or analogue thereof, or the use or the method of any one of the preceding clauses, wherein the variable domain that binds an extracellular part of EGFR is a heavy chain variable region that comprises
at least the CDR3 sequence of the VH of MF3370; MF3755; MF4280 or MF4289 as depicted in FIG. 3 or a CDR3 sequence that differs in at most three, preferably in at most two, preferably in no more than one amino acid from a CDR3 sequence of the VH of MF3370; MF3755; MF4280 or MF4289 as depicted in FIG. 3;
at least the CDR1, CDR2 and CDR3 sequences of the VH of MF3370; MF3755; MF4280 or MF4289 as depicted in FIG. 3; or the CDR1, CDR2 and CDR3 sequences of the VH of MF3370; MF3755; MF4280 or MF4289 as depicted in FIG. 3 with at most three, preferably at most two, preferably at most one amino acid substitutions; or
the sequence of the VH chain of MF3370; MF3755; MF4280 or MF4289 as depicted in FIG. 3; or the amino acid sequence of the VH chain of MF3370; MF3755; MF4280 or MF4289 depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain of MF3370; MF3755; MF4280 or MF4289.
66. The antibody or functional part, derivative and/or analogue thereof, or the use or the method of any one of the preceding clauses, wherein the variable domain that binds EGFR binds an epitope that is located within amino acid residues 420-480 of the human EGFR sequence depicted in FIG. 2.
67. The antibody or functional part, derivative and/or analogue thereof, or the use or the method of any one of the preceding clauses, wherein binding of the variable domain to EGFR is reduced by one or more of the following amino acid residue substitutions I462A; G465A; K489A; I491A; N493A; and C499A in EGFR as compared to an EGFR protein not comprising said substitutions.
68. The antibody or functional part, derivative and/or analogue thereof, or the use or the method of any one of the preceding clauses, wherein the variable domain that binds LGR5 is a heavy chain variable region that comprises
   at least the CDR3 sequence of the VH of MF5790; MF5803; MF5805; MF5808; MF5809; MF5814; MF5816; MF5817; or MF5818 as depicted in FIG. 3 or a CDR3 sequence that differs in at most three, preferably in at most two, preferably in no more than one amino acid from a CDR3 sequence of the VH of MF5790; MF5803; MF5805; MF5808; MF5809; MF5814; MF5816; MF5817; or MF5818 as depicted in FIG. 3;
   at least the CDR1, CDR2 and CDR3 sequences of the VH of MF5790; MF5803; MF5805; MF5808; MF5809; MF5814; MF5816; MF5817; or MF5818 as depicted in FIG. 3; or the CDR1, CDR2 and CDR3 sequences of the VH of MF5790; MF5803; MF5805; MF5808; MF5809; MF5814; MF5816; MF5817; or MF5818 as depicted in FIG. 3 with at most three, preferably at most two, preferably at most one amino acid substitutions; or
   the sequence of the VH chain of MF5790; MF5803; MF5805; MF5808; MF5809; MF5814; MF5816; MF5817; or MF5818 as depicted in FIG. 3; or the amino acid sequence of the VH chain of MF5790; MF5803; MF5805; MF5808; MF5809; MF5814; MF5816; MF5817; or MF5818 depicted in FIG. 3 having at most 15, preferably 1, 2, 3, 4, 5, 6, 7, 8, 9 or 10 and preferably having 1, 2, 3, 4 or 5 amino acid insertions, deletions, substitutions or a combination thereof with respect to the VH chain of MF5790; MF5803; MF5805; MF5808; MF5809; MF5814; MF5816; MF5817; or MF5818.
69. The antibody or functional part, derivative and/or analogue thereof, or the use or the method of any one of the preceding clauses, wherein the variable domain that binds an extracellular part of EGFR comprises a heavy chain variable region comprising the CDR1, CDR2 and CDR3 sequences of a variable region selected from the group consisting of MF3370; MF3755; MF4280 or MF4289 as depicted in FIG. 3 and wherein the variable domain that binds an extracellular part of LGR5 comprises a heavy chain variable region comprising the CDR1, CDR2 and CDR3 sequences of a variable region selected from the group consisting of MF5790; MF5803; MF5805; MF5808; MF5809; MF5814; MF5816; MF5817; or MF5818 as depicted in FIG. 3.
70. The antibody or functional part, derivative and/or analogue thereof, or the use or the method of any one of the preceding clauses, wherein the variable domain that binds an extracellular part of EGFR comprises a heavy chain variable region comprising the CDR1, CDR2 and CDR3 sequences of the variable region of MF3755 as depicted in FIG. 3 and wherein the variable domain that binds an extracellular part of LGR5 comprises a heavy chain variable region comprising the CDR1, CDR2 and CDR3 sequences of the variable region of MF5816 as depicted in FIG. 3.
71. The antibody or functional part, derivative and/or analogue thereof, or the use or the method of any one of the preceding clauses, wherein a VH chain of the variable domain that binds EGFR comprises the amino acid sequence of VH chain MF3370; MF3755; MF4280 or MF4289 as depicted in FIG. 3; or the amino acid sequence of VH chain MF3370; MF3755; MF4280 or MF4289 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH; and wherein a VH chain of the variable domain that binds LGR5 comprises the amino acid sequence of VH chain MF5790; MF5803; MF5805; MF5808; MF5809; MF5814; MF5816; MF5817; or MF5818 as depicted in FIG. 3; or the amino acid sequence of VH chain MF5790; MF5803; MF5805; MF5808; MF5809; MF5814; MF5816; MF5817; or MF5818 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH.
72. The antibody or functional part, derivative and/or analogue thereof, or the use or the method of any one of the preceding clauses, wherein a VH chain of the variable domain that binds EGFR comprises the amino acid sequence of VH chain MF3755 as depicted in FIG. 3; or the amino acid sequence of VH chain MF3755 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH; and wherein a VH chain of the variable domain that binds LGR5 comprises the amino acid sequence of VH chain MF5816 as depicted in FIG. 3; or the amino acid sequence of VH chain MF5816 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH.
73. The antibody or functional part, derivative and/or analogue thereof, or the use or the method of any one of the preceding clauses, wherein a VH chain of the variable domain that binds EGFR comprises the amino acid sequence of VH chain MF3370; MF3755; MF4280 or MF4289 as depicted in FIG. 3; and wherein a VH chain of the variable domain that binds LGR5 comprises the amino acid sequence of VH chain MF5790; MF5803; MF5805; MF5808; MF5809; MF5814; MF5816; MF5817; or MF5818 as depicted in FIG. 3.
74. The antibody or functional part, derivative and/or analogue thereof, or the use or the method of any one of the preceding clauses, wherein a VH chain of the variable domain that binds EGFR comprises the amino acid sequence of VH chain MF3755 as depicted in FIG. 3; and wherein a VH chain of the variable domain that binds LGR5 comprises the amino acid sequence of VH chain MF5816 as depicted in FIG. 3.
75. The antibody or functional part, derivative and/or analogue thereof, or the use or the method of any one of the preceding clauses, wherein both said variable domains that bind EGFR and that bind LGR5 comprise the CDR1, CDR2 and CDR3 regions of the light chain variable region as depicted in FIG. 4b.
76. The antibody or functional part, derivative and/or analogue thereof, or the use or the method of any one of the preceding clauses, wherein both said variable domains that bind EGFR and that bind LGR5 comprise the light chain variable region as depicted in FIG. 4b, which variable light chain region comprises from 0 to 10 amino acid insertions, deletions, substitutions, additions or a combination thereof, wherein the amino acid insertions, deletions and substitutions are not present in the CDR1, CDR2 and CDR3 light chain variable regions
77 The antibody or functional part, derivative and/or analogue thereof, or the use or the method of any one of the preceding clauses, wherein the variable domain that binds LGR5 binds an epitope that is located within amino acid residues 21-118 of the human LGR5 sequence depicted in FIG. 1.
78. The antibody or functional part, derivative and/or analogue thereof, or the use or the method of any one of the preceding clauses, wherein the antibody that binds an extracellular part of EGFR is petosemtamab.
79. A kit of parts comprising an antibody or functional part, derivative and/or analogue thereof as defined in any one of the preceding clauses, an immune checkpoint inhibitor as defined in any one of the preceding clauses and instructions for use of said antibody or functional part, derivative and/or analogue thereof and for use of said immune checkpoint inhibitor.
80. The kit according to clause 79, wherein the instructions for use of the antibody or functional part, derivative and/or analogue thereof comprise instructions for dosing at 1500 mg.
81. The kit according to clause 79 or 80, wherein the instructions for use of the antibody or functional part, derivative and/or analogue thereof comprise instructions for dosing at 1500 mg of once every two weeks.
82. The kit of parts according to any one of clauses 79-81, wherein the kit comprises instructions for use of the antibody or functional part, derivative and/or analogue thereof and the immune checkpoint inhibitor in the treatment of head and neck cancer.
83. The kit of parts according to any one of clauses 79-82, wherein the kit comprises instructions for use of the antibody or functional part, derivative and/or analogue thereof and the immune checkpoint inhibitor in the treatment of head and neck squamous cell cancer.
84. A combination of an antibody or functional part, derivative and/or analogue thereof as defined in any one of clauses 1-78 and an immune checkpoint inhibitor as defined in any one of clauses 1-78 for use in the treatment of cancer in a subject in need thereof.
85. An immune checkpoint inhibitor, as defined in any one of clauses 1-78, for the treatment of cancer in a subject in need thereof, wherein the immune checkpoint inhibitor is for simultaneous or sequential administration with an antibody or functional part, derivative and/or analogue thereof as defined in any one of clauses 1-78.
86. An antibody or functional part, derivative and/or analogue thereof as defined in any one of clauses 1-78, for use in the treatment of cancer in a subject to which an immune checkpoint inhibitor, as defined in any one of clauses 1-78, has been or will be administered.
87. An immune checkpoint inhibitor, as defined in any one of clauses 1-78, for use in the treatment of cancer in a subject to which an antibody or functional part, derivative and/or analogue thereof as defined in any one of clauses 1-78 has been or will be administered.
88. A combination of an antibody or functional part, derivative and/or analogue thereof, as defined in any one of clauses 1-78, instructions for use in the treatment of cancer in a subject of said antibody or functional part, derivative and/or analogue thereof, and instructions for use in the treatment of cancer in a subject of an immune checkpoint inhibitor as defined in any one of clauses 1-78.
89. A combination of an immune checkpoint inhibitor, as defined in any one of clauses 1-78, instructions for use of said inhibitor in the treatment of cancer in a subject and instructions for use in the treatment of cancer in a subject of an antibody or functional part, derivative and/or analogue thereof as defined in any one of clauses 1-78.
90. A combination of clause 88 or 89, wherein the instructions for use comprise the amount of the immune checkpoint inhibitor and the amount of the antibody or functional part, derivative and/or analogue thereof to be used, the dosing interval and cancer to be treated.
91. A method of selecting a subject having a head and neck cancer for treatment with an antibody or functional part, derivative and/or analogue thereof that comprises a variable domain that binds an extracellular part of EGFR according to any one of clauses 1-78 and an immune checkpoint inhibitor according to any one of clauses 1-78, the method comprising: a) determining the Combined Positive Score (CPS) for PD-L1 expression in a sample obtained from the subject; and b) selecting the subject for said treatment if the sample has a CPS for PD-L1 expression of 1 or more, but optionally not more than 100.
92. A method of selecting a subject having a head and neck cancer for treatment with an antibody or functional part, derivative and/or analogue thereof that comprises a variable domain that binds an extracellular part of EGFR according to any one of clauses 1-78 and an immune checkpoint inhibitor according to any one of clauses 1-78, the method comprising: a) determining the Combined Positive Score (CPS) for PD-L1 expression in a sample obtained from the subject; and b) selecting the subject for said treatment if the sample has a CPS for PD-L1 expression of between ≥1 and <20.
93. A method of selecting a subject having a head and neck cancer for treatment with an antibody or functional part, derivative and/or analogue thereof that comprises a variable domain that binds an extracellular part of EGFR according to any one of clauses 1-78 and an immune checkpoint inhibitor according to any one of clauses 1-78, the method comprising: a) determining the Combined Positive Score (CPS) for PD-L1 expression in a sample obtained from the subject; and b) selecting the subject for said treatment if the sample has a CPS for PD-L1 expression of between ≥20 and 100.
94. The method of any one of clauses 91-93, wherein said method further includes determining p16 status on a sample from said subject.
95. The method of clause 94, wherein said sample from said subject is positive for p16 status.
96. The method of clause 94, wherein said sample from said subject is negative for p16 status.
97. A method of establishing whether a subject having a head and neck cancer is likely to respond to treatment with a head and neck cancer for treatment with an antibody or functional part, derivative and/or analogue thereof according to any one of clauses 1-88 that comprises a variable domain that binds an extracellular part of EGFR and an immune checkpoint inhibitor according to any one of clauses 1-78, the method comprising: a) determining the Combined Positive Score (CPS) for PD-L1 expression in a sample obtained from the subject; and b) selecting a sample exhibiting CPS expression of PD-L1 of 1 or more, but optionally not more than 100, thereby establishing that the subject from which the sample is derived is likely to respond to said treatment.
98. A method of establishing whether a subject having a head and neck cancer is likely to respond to treatment with a head and neck cancer for treatment with an antibody or functional part, derivative and/or analogue thereof according to any one of clauses 1-78 that comprises a variable domain that binds an extracellular part of EGFR and an immune checkpoint inhibitor according to any one of clauses 1-78, the method comprising: a) determining the Combined Positive Score (CPS) for PD-L1 expression in a sample obtained from the subject; and b) selecting a sample exhibiting CPS expression of PD-L1 of between ≥1 and <20, thereby establishing that the subject from which the sample is derived is likely to respond to said treatment.
99. A method of establishing whether a subject having a head and neck cancer is likely to respond to treatment with a head and neck cancer for treatment with an antibody or functional part, derivative and/or analogue thereof according to any one of clauses 1-78 that comprises a variable domain that binds an extracellular part of EGFR and an immune checkpoint inhibitor according to any one of clauses 1-78, the method comprising: a) determining the Combined Positive Score (CPS) for PD-L1 expression in a sample obtained from the subject; and b) selecting a sample exhibiting CPS expression of PD-L1 of between ≥20 and 100, thereby establishing that the subject from which the sample is derived is likely to respond to said treatment.
100. The method of any one of clauses 97-99, wherein said method further includes determining p16 status on a sample from said subject.
101. The method of clause 100, wherein said sample from said subject is positive for p16 status.
102. The method of clause 100, wherein said sample from said subject is negative for p16 status.
103. A method of classifying subjects having a head and neck cancer on the basis of the Combined Positive Score for PD-L1 expression prior to treatment with an antibody or functional part, derivative and/or analogue thereof according to any one of clauses 1-78 that comprises a variable domain that binds an extracellular part of EGFR and an immune checkpoint inhibitor according to any one of clauses 1-78, the method comprising: a) determining the Combined Positive Score for PD-L1 expression in a sample obtained from the subject; and b) classifying the subject from which the sample was obtained as eligible for said treatment if the sample exhibits CPS expression for PD-L1 of 1 or more, but optionally not more than 100.
104. A method of classifying subjects having a head and neck cancer on the basis of the Combined Positive Score for PD-L1 expression prior to treatment with an antibody or functional part, derivative and/or analogue thereof according to any one of clauses 1-78 that comprises a variable domain that binds an extracellular part of EGFR and an immune checkpoint inhibitor according to any one of clauses 1-78, the method comprising: a) determining the Combined Positive Score for PD-L1 expression in a sample obtained from the subject; and b) classifying the subject from which the sample was obtained as eligible for said treatment if the sample exhibits CPS expression for PD-L1 of between ≥1 and <20.
105. A method of classifying subjects having a head and neck cancer on the basis of the Combined Positive Score for PD-L1 expression prior to treatment with an antibody or functional part, derivative and/or analogue thereof according to any one of clauses 1-78 that comprises a variable domain that binds an extracellular part of EGFR and an immune checkpoint inhibitor according to any one of clauses 1-78, the method comprising: a) determining the Combined Positive Score for PD-L1 expression in a sample obtained from the subject; and b) classifying the subject from which the sample was obtained as eligible for said treatment if the sample exhibits CPS expression for PD-L1 of between ≥20 and 100.
106. The method of any one of clauses 103-105, wherein said method further includes determining p16 status on a sample from said subject.
107. The method of clause 106, wherein said sample from said subject is positive for p16 status.
108. The method of clause 106, wherein said sample from said subject is negative for p16 status.
109. A method according to any one of clauses 91-108, wherein CPS for PD-L1 expression is determined using IHC.
110. A method according to any one of clauses 91-109, wherein CPS for PD-L1 expression is determined by IHC using clone 22C3.
111. A method according to any one of clauses 91-110, wherein the sample comprises or is a tumor sample or a cancer sample.
112. A method according to any one of clauses 91-111, wherein the cancer comprises or is head and neck cancer.
113. A method according to any one of clauses 91-112, wherein the cancer comprises or is head and neck squamous cell carcinoma (HNSCC).
114. A method according to any one of clauses 91-113, further comprising administering to said subject selected for or eligible for treatment or said subject likely to respond to treatment, an effective amount of an antibody or functional part, derivative and/or analogue thereof that comprises a variable domain that binds an extracellular part of EGFR and an effective amount of an immune checkpoint inhibitor.
115. A method according to any one of clauses 91-114, wherein said subject or sample thereof has a CPS score of between ≥1 and 100, and is positive for p16 status.
116. A method according to any one of clauses 91-114, wherein said subject or sample thereof has a CPS score of between ≥1 and 100, and is negative for p16 status.
117. A method according to any one of clauses 91-114, wherein said subject or sample thereof has a CPS score of between ≥1 and <20, and is positive for p16 status.
118. A method according to any one of clauses 91-114, wherein said subject or sample thereof has a CPS score of ≥1 and <20 and is negative for p16 status.
119. A method according to any one of clauses 91-114, wherein said subject or sample thereof has a CPS score of between ≥20 and 100, and is positive for p16 status.
120. A method according to any one of clauses 91-114, wherein said subject or sample thereof has a CPS score of between ≥20 and 100, and is negative for p16 status.

### EXAMPLES

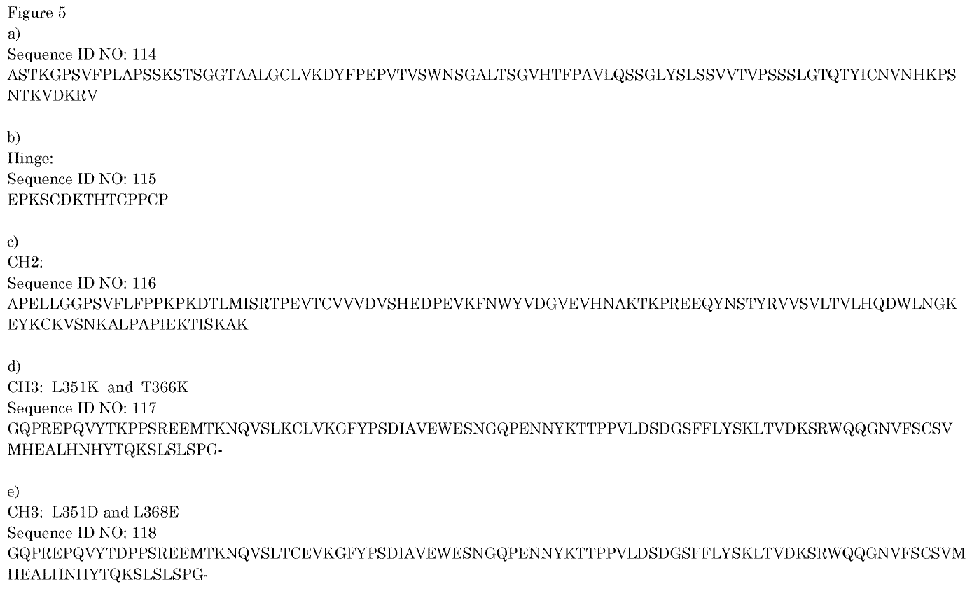

As used herein “MFXXXX” wherein X is independently a numeral 0-9, refers to a Fab comprising a variable domain wherein the VH has the amino acid sequence identified by the 4 digits depicted in FIG. 3. Unless otherwise indicated the light chain variable region of the variable domain typically has a sequence of FIG. 4b. The light chain in the examples has a sequence as depicted in FIG. 4a. “MFXXXX VH” refers to the amino acid sequence of the VH identified by the 4 digits. The MF further comprises a constant region of a light chain and a constant region of a heavy chain that normally interacts with a constant region of a light chain. The VH/variable region of the heavy chains differs and typically also the CH3 region, wherein one of the heavy chains has a KK mutation of its CH3 domain and the other has the complementing DE mutation of its CH3 domain (see for reference PCT/NL2013/050294 (published as WO2013/157954) and FIGS. 5d and 5e. Bispecific antibodies in the examples have an Fe tail with a KK/DE CH3 heterodimerization domain, a CH2 domain and a CH1 domain as indicated in FIG. 5, a common light chain as indicated in FIG. 4a and a VH as specified by the MF number. For example a bispecific antibody indicated by MF3755×MF5816 has the above general sequences and a variable domain with a VH with the sequence of MF3755 and a variable domain with a VH with the sequence of MF5816.

The amino acid sequences of the various heavy chain variable regions (VH) are indicated in FIG. 3. Bispecific antibodies EGFR/LGR5, MF3755×MF5816; comprising heavy chain variable regions MF3755 and MF5816 and a common light chain and including modifications for enhanced ADCC from afucosylation, among other LGR5 and EGFR combinations as depicted in FIG. 3 have been shown to be effective in WO2017/069628.

### Example 1. Generation of Bispecific Antibodies

Bispecific antibodies were generated by transient co-transfection of two plasmids encoding IgG with different VH domains, using a proprietary CH3 engineering technology to ensure efficient heterodimerization and formation of bispecific antibodies. The common light chain is also co-transfected in the same cell, either on the same plasmid or on another plasmid. In our applications (e.g. WO2013/157954 and WO2013/157953; incorporated herein by reference) we have disclosed methods and means for producing bispecific antibodies from a single cell, whereby means are provided that favor the formation of bispecific antibodies over the formation of monospecific antibodies. These methods can also be favorably employed in the present invention. Specifically, preferred mutations to produce essentially only bispecific full length IgG molecules are amino acid substitutions at positions 351 and 366, e.g. L351K and T366K (numbering according to EU numbering) in the first CH3 domain (the ‘KK-variant’ heavy chain) and amino acid substitutions at positions 351 and 368, e.g. L351D and L368E in the second CH3 domain (the ‘DE-variant’ heavy chain), or vice versa (see FIGS. 5d and 5e). It was previously demonstrated in the mentioned applications that the negatively charged DE-variant heavy chain and positively charged KK-variant heavy chain preferentially pair to form heterodimers (so-called ‘DEKK’ bispecific molecules). Homodimerization of DE-variant heavy chains (DE-DE homodimers) or KK-variant heavy chains (KK-KK homodimers) hardly occurs due to strong repulsion between the charged residues in the CH3-CH3 interface between identical heavy chains.

VH genes of variable domain that bind LGR5 described above were cloned into the vector encoding the positively charged CH3 domain. The VH genes of variable domain that bind EGFR such as those disclosed in WO 2015/130172 (incorporated herein by reference) were cloned into vector encoding the negatively charged CH3 domain. Suspension growth-adapted 293F Freestyle cells were cultivated in T125 flasks on a shaker plateau until a density of 3.0×10e6 cells/ml. Cells were seeded at a density of 0.3-0.5×10e6 viable cells/ml in each well of a 24-deep well plate. The cells were transiently transfected with a mix of two plasmids encoding different antibodies, cloned into the proprietary vector system. Seven days after transfection, the cellular supernatant was harvested and filtered through a 0.22 μM filter (Sartorius). The sterile supernatant was stored at 4° C. until purification of the antibodies.

### Example 2: IgG Purification and Quantification

Purifications were performed under sterile conditions in filter plates using Protein-A affinity chromatography. First, the pH of the medium was adjusted to pH 8.0 and subsequently, IgG-containing supernatants were incubated with protein A Sepharose CL-4B beads (50% v/v) (Pierce) for 2 hrs at 25° C. on a shaking platform at 600 rpm. Next, the beads were harvested by filtration. Beads were washed twice with PBS pH 7.4. Bound IgG was then eluted at pH 3.0 with 0.1 M citrate buffer and the eluate was immediately neutralized using Tris pH 8.0. Buffer exchange was performed by centrifugation using multiscreen Ultracel 10 multiplates (Millipore). The samples were finally harvested in PBS pH7.4. The IgG concentration was measured using Octet. Protein samples were stored at 4° C.

To determine the amount of IgG purified, the concentration of antibody was determined by means of Octet analysis using protein-A biosensors (Forte-Bio, according to the supplier's recommendations) using total human IgG (Sigma Aldrich, cat. Nr. 14506) as standard.

The following bispecific antibodies are suitable for use in this example and for use in the methods of the invention: MF3370×MF5790, MF3370×5803, MF3370×5805, MF3370×5808, MF3370×5809, MF3370×5814, MF3370×5816, MF3370×5817, MF3370×5818, MF3755×MF5790, MF3755×5803, MF3755×5805, MF3755×5808, MF3755×5809, MF3755×5814, MF3755×5816, MF3755×5817, MF3755×5818, MF4280×MF5790, MF4280×5803, MF4280×5805, MF4280×5808, MF4280×5809, MF4280×5814, MF4280×5816, MF4280×5817, MF4280×5818, MF4289×MF5790, MF4289×5803, MF4289×5805, MF4289×5808, MF4289×5809, MF4289×5814, MF4289×5816, MF4289×5817, and MF4289×5818. Each bispecific antibody comprises two VH as specified by the MF numbers capable of binding EGFR and LGR5 respectively, further comprises an Fe tail with a KK/DE CH3 heterodimerization domain as indicated by SEQ ID NO: 117 (FIG. 5d) and SEQ ID NO: 118 (FIG. 5e), respectively, a CH2 domain as indicated by SEQ ID NO: 116 (FIG. 5c), a hinge region as indicated by SEQ ID NO:115, and a CH1 domain as indicated by SEQ ID NO:114 (FIG. 5a), a common light chain as indicated by SEQ ID NO: 107 (FIG. 4).

### Example 3: Dose Expansion in Patients Having HNSCC with Petosemtamab and Pembrolizumab

A combination of petosemtamab and pembrolizumab will be explored first in patients with HNSCC. HNSCC patients who are in first-line systemic treatment for recurrent/metastatic disease are eligible. The combination will start with a full dose of both therapeutic agent.

### Study Design

A phase 1 open-label multicenter study was performed with an initial dose escalation part to determine the recommended phase 2 dose (RP2D) of petosemtamab, an anti-EGFR×anti-LGR5 bispecific antibody, for solid tumors in mCRC patients with a starting dose of 5 mg flat dose. Since the RP2D was established at 1500 mg Q2W, the antibody is further evaluated in an expansion part of the study, including in patients diagnosed with Head and Neck cancer, including squamous cell carcinoma of the head and neck (SCCHN). Safety, PK, immunogenicity and preliminary antitumor activity of the antibody is characterized in all patients, and biomarker analyses, including EGFR and LGR5 status is performed.

### Inclusion Criteria.

1. Signed informed consent form (ICF) before initiation of any study procedures
2. Age ≥18 years at signing of informed consent.
3. Histologically or cytologically confirmed solid HNSCC tumors with evidence of metastatic or locally advanced disease not amenable to standard therapy with curative intent:
   Expansion cohorts: patients with locally advanced unresectable or metastatic disease for the following indications:
      FIRST LINE HNSCC: patients eligible to receive pembrolizumab as first-line monotherapy with tumors expressing PD-L1, CPS≥1, as determined by an FDA-approved test in the US, or by an approved equivalent test in other countries; patients should not have previous systemic therapy administered in the recurrent or metastatic setting, although previous systemic therapy as part of multimodal treatment for locally advance disease is allowed if ended ≥6 months prior to signing the ICF or if progressive disease was ≥6 months after the last platinum-containing therapy dose. The eligible HNSCC primary tumor locations are oropharynx, oral cavity, hypopharynx, and larynx. Previous treatments with anti-PD-(L)1 or anti-EGFR therapies are not allowed.
4. Documentation of p16 status (positive or negative) by local laboratory IHC for patients with primary oropharyngeal cancer.
5. A baseline new tumor sample (formalin-fixed paraffin-embedded [FFPE]) from a metastatic or primary site. If the patient has an available tumor sample as an FFPE block with sufficient material (at least 20 slides with >20% tumor content) and has not received further anticancer treatment since sample collection, a new tumor biopsy at baseline is not necessary. Archival FFPE slides are not acceptable. Primary tumor material of HNSCC is only acceptable if the patient has not been treated with anti-EGFR or anti-human epidermal growth factor receptor (HER)-2 therapies.
6. Amenable for biopsy
7. Measurable disease as defined by RECIST version 1.1 by radiologic methods
8. Eastern Cooperative Oncology Group (ECOG) performance status of 0 or 1
9. Life expectancy ≥12 weeks, as per investigator
10. Left ventricular ejection fraction (LVEF)≥50% by echocardiogram (ECHO) or multigated acquisition scan (MUGA)
11. Adequate organ function:
   ANC≥1.5×10⁹/L
   Hemoglobin ≥9 g/dL
   Platelets ≥100×10⁹/L
   Corrected total serum calcium within normal ranges
   Serum magnesium within normal ranges (or corrected with supplements)
   Alanine aminotransferase (ALT), aspartate aminotransferase (AST)≤2.5× upper limit of normal (ULN) and total bilirubin ≤1.5×ULN (unless due to known Gilbert's syndrome who are excluded if total bilirubin >3.0×ULN or direct bilirubin >1.5×ULN); in cases of liver involvement, ALT/AST≤5×ULN and total bilirubin ≤2×ULN will be allowed, unless due to known Gilbert's syndrome when total bilirubin ≤3.0×ULN or direct bilirubin ≤1.5×ULN will be allowed or hepatocellular carcinoma [Child-Pugh class A] when total bilirubin <3 mg/dL will be allowed.
   Serum creatinine ≤1.5×ULN or creatinine clearance ≥60 mL/min calculated according to the Cockroft and Gault formula or Modification of Diet in Renal Disease (MDRD) formula for patients aged >65 years
   Serum albumin ≥3 g/dL
   International Normalized Ratio (INR) or Prothrombin Time (PT)≤1.5×ULN unless patient is receiving anticoagulant therapy and in therapeutic range of intended used anticoagulant
   Activated Partial Thromboplastin Time (APTT) or PTT≤1.5×ULN unless patient is receiving anticoagulant therapy and is in therapeutic range of intended used anticoagulant
12. Willing to undergo testing for human immunodeficiency virus (HIV) if not tested within 6 months (upon study entry). HIV-positive patients are eligible provided the CD4+ count is ≥300/μL, viral load is undetectable, and the patient is currently receiving highly active antiretroviral therapy (HAART).

### Exclusion Criteria

1. Central nervous system metastases that are untreated or symptomatic, or require radiation, surgery, or continued steroid therapy to control symptoms within 14 days of study entry
2. Known leptomeningeal involvement
3. Participation in another clinical study or treatment with any investigational drug within 4 weeks prior to study entry
4. Any systemic anticancer therapy within 4 weeks or 5 half-lives, whichever is longer, of the first dose of study treatment. For cytotoxic agents that have major delayed toxicity (eg, mitomycin C, nitrosoureas), or anticancer immunotherapies, a washout period of 6 weeks is required
5. Requirement for immunosuppressive medication (eg, methotrexate, cyclophosphamide)
6. Major surgery or radiotherapy within 3 weeks of the first dose of study treatment. Patients who received prior radiotherapy to ≥25% of bone marrow are not eligible, irrespective of when it was received.
7. Persistent Grade >1 clinically significant toxicities related to prior antineoplastic therapies (except for alopecia); stable sensory neuropathy ≤Grade 2 National Cancer Institute-Common Terminology Criteria for Adverse Events (NCI-CTCAE) v4.03 or v5.0 is allowed.
8. History of hypersensitivity reaction or any toxicity attributed to human proteins or any of the excipients, such as from pembrolizumab or petosemtamab that warranted permanent cessation of these agents
9. Uncontrolled hypertension (systolic BP>150 mmHg and/or diastolic BP>100 mmHg) with appropriate treatment, or unstable angina.
10. History of congestive heart failure of Class II-IV New York Heart Association (NYHA) criteria, or serious cardiac arrhythmia requiring treatment (except atrial fibrillation, paroxysmal supraventricular tachycardia)
11. History of myocardial infarction within 6 months of study entry
12. History of prior malignancies with the exception of excised cervical intraepithelial neoplasia or nonmelanoma skin cancer, or curatively treated cancer deemed at low risk for recurrence with no evidence of disease for at least 3 years
13. Current dyspnea at rest of any origin, or other diseases requiring continuous oxygen therapy
14. Patients with a history of interstitial lung disease (ILD) (eg, pneumonitis or pulmonary fibrosis), or evidence of ILD on baseline chest computerized tomography (CT) scan
15. Current serious illness or medical conditions including, but not limited to, uncontrolled active infection, clinically significant pulmonary, metabolic or psychiatric disorders
16. Patients with the following infectious diseases:
   Active hepatitis B surface antigen infection (HBsAg positive) without receiving antiviral treatment. Note:
      Patients with active hepatitis B (HBsAg positive) must receive antiviral treatment with lamivudine, tenofovir, entecavir, or other antiviral agents, starting at least ≥7 days before the initiation of study treatment.
      Patients with antecedents of hepatitis B (anti-HBc positive, HBsAg and hepatitis B virus [HBV]-DNA negative) are eligible.
   Positive test for hepatitis C virus (HCV) ribonucleic acid (RNA). Note: Patients in whom HCV infection resolved spontaneously (ie, positive HCV antibodies without detectable HCV-RNA) or who achieved a sustained response after antiviral treatment and show absence of detectable HCV RNA≥6 months (with the use of IFN-free regimens) or ≥12 months (with the use of IFN-based regimens) after cessation of antiviral treatment are eligible.
17. Patients with current cirrhotic status of Child-Pugh class B or C; known fibrolamellar HCC, sarcomatoid HCC, or mixed cholangiocarcinoma and HCC
18. Pregnant or breastfeeding patients; patients of childbearing potential must use highly effective contraception methods prior to study entry, for the duration of study participation, and for 6 months after the last dose of petosemtamab.
19. Having a diagnosis of immunodeficiency, or is receiving systemic steroid therapy or any form of immunosuppressive therapy within 7 days prior to the first dose. Corticosteroids use as premedication for allergic reactions or IRRs specified in the protocol are allowed.
20. Having an active autoimmune disease that has required systemic immune suppressive treatment in the past 2 years; replacement therapy (eg, thyroxine, insulin, or physiologic corticosteroid replacement therapy for adrenal or pituitary insufficiency, etc.) is not considered immune suppressive treatment.
21. Having had an allogeneic tissue/solid organ transplant
22. Patient has a primary tumor site of nasopharynx (any histology).

### Dose Expansion

In the expansion part, petosemtamab is administered at the RP2D in patients having head and neck cancer, in particular SCCHN. With RP2D defined at 1500 mg Q2W, additional patients will be treated with this dose and schedule to further characterize safety, tolerability, PK and immunogenicity of antibody, and to perform a preliminary assessment of antitumor activity and biomarker evaluations. Alternatively, petosemtamab is used at 1100 mg flat dose Q2W or dosing which achieves human receptor occupancy for both EGFR and LGR5 of at least 95% or at least 99%.

Antibody treatment in patients with head and neck cancer, in particular SCCHN, is explored for anti-tumor activity. The overall safety of the drug will also be interrogated.

### Investigational Therapy and Regimen

Petosemtamab, an anti-EGFR×anti-LGR5 bispecific antibody, is formulated as a clear liquid solution for IV infusion. IV infusion is performed every 2 weeks using standard infusion procedures, with a starting dose of 5 mg (flat dose), and with a recommended phase 2 dose of 1500 mg (flat dose). Dose escalation was halted once the RP2D had been reached. Infusions must be administered over a minimum of 4 hours during Cycle 1. Subsequent infusions after Cycle 1 can be reduced to 2 hours at the investigator's discretion and in the absence of IRRs. A cycle is considered 4 weeks.

### Pembrolizumab Administration

Pembrolizumab dosing is 400 mg Q6W, but can also be 200 mg Q3W. Administration is through 30-minute intravenous infusion and/or following the label instructions in each country where administered. Injection is of 100 mg/4 mL (25 mg/mL) solution in a single-dose vial.

### Premedication

During Cycle 1, all infusions will be administered over a period of at least 4 hours with the following premedication regimen: 24 hours before the start of the infusion, 8 mg of dexamethasone PO will be administered 1 hour before the start of the infusion, each patient will receive dexamethasone 20 mg IV, Dexchlorpheniramine 5 mg IV or diphenhydramine 50 mg PO or chlorpheniramine 10 mg IV, Ranitidine 50 mg IV or 150 mg PO and Paracetamol 1 g IV or 650 mg PO.

### Treatment Duration

Study treatment is administered until confirmed progressive disease (as per RECIST 1.1), unacceptable toxicity, withdrawal of consent, patient non-compliance, investigator decision (e.g. clinical deterioration), or antibody interruption >6 consecutive weeks. Patients are followed up for safety for at least 30 days following the last antibody infusion and until recovery or stabilization of all related toxicities, and for disease progression and survival status for 12 months.

### Efficacy Assessments

Tumor assessment is based on CT/MRI with contrast per RECIST 1.1 (Eisenhauer et al., 2009 Eur J Cancer 45:228-247), every 8 weeks after treatment start. Objective responses must be confirmed at least 4 weeks after first observation. Bone scans are performed as clinically indicated for patients with bone metastases at baseline or suspected lesions on study. Circulating blood tumor markers, including carcinoembryonic antigen (CEA), are evaluated at screening and on Day 1 of each cycle.

### Concomitant Medications

Key permitted medications are as follows: Paracetamol/acetaminophen, antihistamines, and corticosteroids are permitted for as premedication treatment regimen but can also be administered in the event of IRRs, hypersensitivity or allergic reactions, according to standard local clinical practice. All medication necessary for the patient's safety and well-being, and which is not expected to interfere with evaluation of the study drug, may be given at the investigator's discretion. Concurrent radiation treatment during this study for symptom control without evidence of progression.

Key prohibited medications are as follows: Concomitant chronic oral corticosteroids (>10 mg/day prednisone equivalent), tumor necrosis factor (TNF)-alpha inhibitors, anti-T-cell antibodies, or other immunosuppressive medication. Any investigational drug or other anticancer therapy during the study or within 4 weeks (or, if known, 5 half-lives), whichever is longer, prior to the first dose of study treatment. For cytotoxic agents that have major delayed toxicity (eg, mitomycin C, nitrosoureas), or anticancer immunotherapies, a washout period of 6 weeks is required. Major surgery or radiotherapy within 3 weeks prior to the first dose of study treatment, or prior radiotherapy to ≥25% of bone marrow.

### Example 4. CPS Scoring in HNSCC Cancer Patients

Prior to commencing combination therapy, under the clinical trial protocol prescreening to detect tumors that express PD-L1 is required for patients with HNSCC. PD-L1 testing must be performed in a CLIA-certified laboratory (or equivalent) and by an FDA-approved test in the US or by an equivalent approved test in other countries. To be eligible for inclusion, patients with HNSCC must have histologically confirmed CPS≥1; CPS is defined as the number of PD L1 positive tumor cells, lymphocytes, and macrophages divided by the total number of tumor cells, multiplied by 100. If local testing is not available, then testing will be performed in a central laboratory qualified to perform such tests.

### Reagent Preparation

The following reagents must be prepared prior to staining. EnVision FLEX Target Retriev al Solution, Low pH (50×). Prepare a sufficient quantity of 1× Target Retrieval Solution, Low pH by diluting Target Retrieval Solution, Low pH (50×) 1:50 using distilled or deionized water (reagent-quality water); the pH of 1× Target Retrieval Solution must be 6.1±0.2. 1× Target Retrieval Solution pH below 5.9 may give erroneous results. One 30 mL bottle of Target Retrieval Solution, Low pH (50×) diluted 1:50 will provide 1.5 L of 1× reagent, sufficient to fill one PT Link tank which will treat up to 24 slides per use. Discard 1× Target Retrieval Solution after three uses and do not use after 5 days following dilution.

EnVision FLEX Wash Buffer (20×). Prepare a sufficient quantity of Wash Buffer by diluting Wash Buffer (20×) 1:20 using distilled or deionized water (reagent-quality water) for the wash steps. Store unused 1× solution at 2-8° C. for no more than one month. Discard buffer if cloudy in appearance.

DAB+ Substrate-Chromogen Solution. This solution should be mixed thoroughly prior to use. Any precipitate developing in the solution does not affect staining quality. To prepare DAB+ Substrate-Chromogen Solution, add 1 drop of Liquid DAB+ Chromogen per mL of DAB+ Substrate Buffer and mix. Prepared Substrate-Chromogen is stable for 5 days if stored in the dark at 2-8° C.

### Specimen Preparation

Tissue specimens must be handled to preserve the tissue for IHC staining. Standard methods of tissue processing should be used for all specimens. Paraffin-embedded Specimens (FFPE) are suitable for use. Alternative fixatives have not been validated and may give erroneous results. Fixation time for 12-72 hours in 10% neutral buffered formalin (NBF) is recommended. Fixation times of ≤3 hours should be avoided. Specimens should be blocked into a thickness of 3 or 4 mm, fixed in formalin and dehydrated and cleared in a series of alcohols and xylene, followed by infiltration with melted paraffin. The paraffin temperature should not exceed 60° C. NSCLC FFPE tissue blocks which are 5 years or older may result in a loss of PD-L1 immunoreactivity.

Tissue specimens should be cut into sections of 4-5 μm. After sectioning, tissues should be mounted on Dako FLEX IHC microscope slides (Code K8020), or Superfrost Plus slides and then placed in a 58±2° C. oven for 1 hour.

Cut Section Storage Recommendation. To preserve antigenicity, tissue sections, once mounted on slides, should be held in the dark at 2-8° C. (preferred), or at room temperature up to 25° C. Slide storage and handling conditions should not exceed 25° C. at any point post-mounting to ensure tissue integrity and antigenicity.

HNSCC cut sections must be stained within 6 months when stored at 2-8° C. (preferred), or within 4 months when stored at 25° C.

### Staining Procedure on the Autostainer Link 48 Solution.

All reagents should be equilibrated to room temperature (20-25° C.) prior to immunostaining. Likewise, all incubations should be performed at room temperature. Do not allow tissue sections to dry during the staining procedure. Dried tissue sections may display increased nonspecific staining. All of the required steps and incubation times for staining are preprogrammed in the Dako Link software.

### Staining Protocol

The PD-L1 IHC 22C3 pharmDx staining protocol is selected from the options in the Dako Link drop down menu. All of the required steps and incubation times for staining are preprogrammed in the Autostainer Link 48.
Step 1: Deparaffinization, Rehydration and Target Retrieval (3-in-1) Procedure Set PT Link (Code PT100/PT101/PT200) Preheat and Cool to 65° C. Set Heat to 97° C. for 20 minutes. Fill PT Link tanks with 1.5 L per tank of Target Retrieval Solution, Low pH, 1× working solution to cover the tissue sections. Preheat the Target Retrieval Solution to 65° C. Immerse Autostainer racks containing mounted, FFPE tissue sections into the pre-heated Target Retrieval Solution, Low pH, (1× working solution) in PT Link tank. Incubate for 20 minutes at 97° C. When target retrieval incubation has been completed and the temperature has cooled to 65° C., remove each Autostainer slide rack with the slides from the PT Link tank and immediately place the Autostainer rack with slides into a tank (e.g., PT Link Rinse Station, Code PT10⁹) containing diluted, room temperature Wash Buffer (Code K8007). Incubate slides in diluted, room temperature Wash Buffer for 5 minutes.
Step 2: Staining Procedure. After deparaffinization, rehydration and target retrieval (3-in-1) procedure, the Autostainer racks with slides are placed on Autostainer Link 48. The instrument will perform the staining process by applying the appropriate reagent, monitoring the incubation time and rinsing slides between reagents. The reagent times are preprogrammed in the Dako Link software.
Step 3: Counterstain. Slides should be counterstained for 5 minutes with Hematoxylin (Code K8008). The Hematoxylin incubation time is preprogrammed in the protocol.
Step 4: Mounting. Non-aqueous, permanent mounting media is required.

### Quality Control

Reagents in PD-L1 IHC 22C3 pharmDx have been quality controlled by immunohistochemistry using the target retrieval and staining procedures. Quality controls should be included in each staining run. These quality controls include: a H&E stained patient tissue specimen; lab-supplied positive and negative control tissues; and a Dako-supplied Control Cell Line Slide.

Assay Verification. Prior to initial use of a staining system in a diagnostic procedure, the user should verify the assay's performance by testing it on a series of lab-supplied tissues with known IHC performance characteristics representing known positive and negative tissues. These quality control procedures should be repeated for each new antibody lot, or whenever there is a change in assay parameters.

### Scoring Interpretation.

All viable tumor cells on the entire tissue section must be evaluated and included in PD-L1 expression assessment. PD-L1 expression is determined by CPS, which is the number of PD-L1 staining cells (tumor cells, lymphocytes, macrophages) divided by the total number of viable tumor cells, multiplied by 100. Distinction of viable tumor cells, lymphocytes, and macrophages is essential for accurate denominator estimation. Although the result of the calculation can exceed 100, the maximum score is defined as CPS 100. Slide evaluation must be performed by a pathologist using a light microscope. For evaluation of the immunohistochemical staining, an objective of 10-20× magnification is appropriate. For determination of PD-L1 expression, an objective of 20× magnification is required. By definition, PD-L1 staining cells are:
Tumor cells with convincing partial or complete linear membrane staining (at any intensity) that is perceived distinct from cytoplasmic staining and
Lymphocytes and macrophages (mononuclear inflammatory cells, MICs) within the tumor nests and/or adjacent supporting stroma with convincing membrane and/or cytoplasmic staining (at any intensity). MICs must be directly associated with the response against the tumor.

For each staining run, slides should be examined in the order presented in Table 2 to determine the validity of the staining run and enable assessment of the staining of the sample tissue. Examine patient specimens stained with PD-L1 and the negative control reagent from PD-L1 IHC 22C3 pharmDx when evaluating PD-L1 expression. Specimens stained with negative control reagent must have 0 specific staining and ≤1+ nonspecific staining.

HNSCC—CPS Interpretation. A minimum of 100 viable tumor cells must be present in the PD-L1 stained slide for the specimen to be considered adequate for PD-L1 evaluation. The CPS denominator includes all viable invasive tumor cells (PD-L1 staining and non-staining). All immune cells, benign cells, necrotic or non-viable tumor cells, carcinoma in situ, stromal cells (including fibroblasts), and necrotic cells and/or cellular debris are excluded. Table 1 provides details about which tissue elements are included in and excluded from the CPS numerator in HNSCC

TABLE 1
CPS Numerator Inclusion/Exclusion Criteria

| Tissue | | |
|---|---|---|
| Elements | Included in the Numerator | Excluded from the Numerator |
| Tumor Cells | Convincing partial or complete | Non-staining tumor cells |
| | linear membrane staining (at | Tumor cells with only |
| | any intensity) of viable invasive | cytoplasmic staining |
| | tumor cells | |
| Immune Cells | Membrane and/or cytoplasmic* | Non-staining MICs |
| | staining (at any intensity) of | MICs (including lymphoid |
| | mononuclear inflammatory cells | aggregates) associated with |
| | (MICs) within tumor nests and | ulcers or other inflammatory |
| | adjacent supporting stroma:** | processes |
| | Lymphocytes (including | MICs associated with |
| | lymphocyte aggregates) | carcinoma in situ |
| | Macrophages*** | MICs associated with benign |
| | Only MICs directly associated | structures |
| | with the response to the tumor | Neutrophils, eosinophils, and |
| | are scored | plasma cells |
| Other Cells | Not included | Carcinoma in situ |
| | | Benign cells |
| | | Stromal cells (including |
| | | fibroblasts) |
| | | Necrotic cells and/or cellular |
| | | debris |

*In MICs, membrane and cytoplasmic staining are often indistinguishable due to high nuclear to cytoplasmic ratio. Therefore, membrane and/or cytoplasmic staining of MICs is included in the score.
**Adjacent MICs are defined as being within the same 20x field as the tumor. However, MICs that are NOT directly associated with the response to the tumor should be excluded.
***Macrophages and histiocytes are considered the same cells

TABLE 2

| Specimen | Rationale | Requirements |
|---|---|---|
| H&E | A hematoxylin and | The PD-L1 IHC 22C3 pharmDx and H&E |
| (Lab- | eosin (H&E) stain of | stain should be performed on serial |
| supplied) | the tissue specimen is | sections from the same paraffin block of |
| | evaluated first to | the specimen. |
| | assess tissue histology | Tissue specimens should be intact, well |
| | and preservation | preserved, and should confirm tumor |
| | quality. | indication. |
| Control slide | The Control Cell Line | One CCLS should be stained with the PD- |
| (supplied | Slide (CCLS) stained | L1 Primary Antibody in each staining run. |
| with kit) | with the PD-L1 | NCI-H226 (PD-L1-positive control cell line) |
| | primary antibody from | acceptance criteria: |
| | PD-L1 IHC 22C3 | Cell membrane staining of ≥70% of cells; ≥2+ |
| | pharm Dx should be | average staining intensity; Non-specific |
| | examined to ascertain | staining <1+ intensity. |
| | that all reagents are | MCF-7 (PD-L1- NC cell line) acceptance |
| | functioning properly | criteria: No specific staining; Non-specific |
| | The CCLS contains the | staining <1+ intensity. Note that staining |
| | PD-L1-positive cell line | of a few cells in the MCF-7 cell pellet may |
| | pellet and PD-L1- | occasionally be observed. The following |
| | negative cell line pellet | acceptance criteria are applicable: the |
| | | presence of ≤10 total cells with distinct |
| | | plasma membrane staining, or cytoplasmic |
| | | staining with ≥1+ intensity within the |
| | | boundaries of the MCF-7 cell pellet are |
| | | acceptable. If either of the CCLS does not |
| | | meet these criteria, all results with the |
| | | patient specimens should be considered |
| | | invalid. |
| Positive | The PC Tissue Slides | Controls should be biopsy/surgical |
| Control (PC) | stained with both PD- | specimens of the same tumor indication as |
| Tissue | L1 primary antibody | the patient specimen, fixed, processed and |
| Slides | and NC Reagent | embedded as soon as possible in the same |
| (Lab- | should be examined | manner as the patient sample(s). Use well- |
| supplied) | next. These slides | preserved specimens for interpretation of |
| | verify that the fixation | staining results as necrotic or degenerated |
| | method and epitope | cells often demonstrate non-specific |
| | retrieval process are | staining. The tissues selected for use as the |
| | effective. Known | positive tissue controls should give weak to |
| | positive tissue controls | moderate positive staining when stained |
| | should only be utilized | with PD-L1 to aid in detection of subtle |
| | for monitoring the | changes in assay sensitivity. |
| | correct performance of | Two positive tissue control slides should be |
| | processed tissues and | included in each staining run. |
| | test reagents, NOT as | Slide stained with PD-L1: Presence of |
| | an aid in formulating a | brown plasma membrane staining should |
| | specific diagnosis of | be observed. Non-specific staining should |
| | patient samples. | be ≤1+. |
| | | Slide stained with NC Reagent: No |
| | | membrane staining. Nonspecific staining |
| | | should be ≤1+. |
| | | If the positive tissue controls fail to |
| | | demonstrate appropriate positive staining, |
| | | results with the test specimens should be |
| | | considered invalid. |
| Negative | The NC Tissue Slides | Controls should be biopsy/surgical |
| Control | (known to be PD-L1 | specimens of the same tumor indication as |
| (NC) Tissue | negative) stained with | the patient specimen, fixed, processed and |
| Slides (Lab- | both PD-L1 primary | embedded as soon as possible in the same |
| supplied) | antibody and NC | manner as the patient sample(s). Two |
| | Reagent should be | negative tissue control slides should be |
| | examined next to | included in each staining run. Slide |
| | verify the specificity of | stained with PD-L1: No membrane |
| | the labeling of the | staining in tumor cells. Non-specific |
| | target antigen by the | staining should be ≤1+. |
| | primary antibody. | Slide stained with NC Reagent: No |
| | Alternatively, negative | membrane staining. Nonspecific staining |
| | portions of the PC | should be ≤1+. |
| | Tissue may serve as | If specific cell membrane staining occurs in |
| | the NC Tissue, but this | the NC Tissue Slides, results with the |
| | should be verified by | patient specimen should be considered |
| | the user. | invalid. |
| Tonsil | Use human tonsil | Strong positive staining should be detected |
| Control | tissue fixed, processed | in portions of the crypt epithelium and |
| Tissue | and embedded in a | weak to moderate staining of the follicular |
| (optional) | manner similar to the | macrophages in the germinal centers. |
| (Lab- | patient sample(s) as an | Negative staining should be observed in |
| supplied) | additional control | endothelium, fibroblasts as well as surface |
| | material to verify | epithelium |
| | sensitivity, specificity | |
| | and nonspecific | |
| | background staining of | |
| | the assay | |
| Patient | Examine patient | Absence of cell membrane staining verifies |
| tissue slide | specimens stained with | the specific labeling of the target antigen |
| stained | the NCR from PD-L1 | by the primary antibody. Non-specific |
| using the | IHC 22C3 pharmDx. | staining should be ≤1+. |
| NC Reagent | NCR is used in place of | |
| (NCR) slide | the primary antibody | |
| stained | and aids in | |
| using the | interpretation of | |
| NCR | specific staining at the | |
| | antigen site. | |
| Patient | Examine the entire | Positive staining intensity should be |
| tissue slide | slide of the patient | assessed within the context of any |
| stained | specimens stained with | nonspecific background staining observed |
| using the | the PD-L1 primary | on the patient's NCR slide in the same run. |
| PD-L1 | antibody from PDL1 | As with any IHC test, a negative result |
| primary | IHC 22C3 pharmDx | means that the antigen was not detected, |
| antibody | last. | not necessarily that the antigen was |
| tissue slide | | absent in the cells/tissue assayed. All |
| stained | | viable tumor cells on the entire PD-L1 |
| using the | | stained patient slide must be evaluated |
| PD-L1 | | and included in the PD-L1 scoring |
| primary | | assessment. A minimum of 100 viable |
| antibody | | tumor cells must be present for the |
| | | specimen to be considered adequate for |
| | | PD-L1 evaluation |

### Example 5. EGFR Scoring Via IHC

The EGFR pharmDx™ assay is a qualitative immunohistochemical (IHC) kit system to identify epidermal growth factor receptor (EGFR) expression in normal and neoplastic tissues routinely-fixed for histological evaluation. EGFR pharmDx specifically detects the EGFR (HER1) protein in EGFR-expressing cells.

The EGFR pharmDx™ assay uses the EGFR antibody, clone 2-18C9 (2-18C9) to detect EGFR protein. Clone 2-18C9 has been tested for reactivity against cell lines expressing EGFR, HER2, HER3 and HER4. In Western blots of SKBR3 and A431 cell lysates, 2-18C9 recognized a 170 kD band which is consistent with the known molecular weight of EGFR. Clone 2-18C9 has also been found to recognize the EGFRvIII (145 kD) form of the receptor in immunohistochemistry, flow cytometry and Western blotting of EGFRvIII transfected cell lines. In Western blotting experiments, 2-18C9 was unreactive with HER2 positive CAMA-1 cell lysates, HER3-transformed E. coli BL-21 protein extracts and CHO-HER4 transfected cell lysates. Additionally, Chinese Hamster Ovary (CHO) transfectants expressing myc (vector tag), either alone or coexpressed with one of the HER family members, were grown in chamber slides that were formalin-fixed and paraffin-embedded, and stained with anti-myc and 2-18C9. The myc antibody stained all five CHO transfectants, whereas 2-18C9 only stained the CHO cells transfected with HER.

EGFR scoring is performed using the Dako EGFR pharmDx™ user protocol according to the manufacturer's instructions and recommendations. See the world wide web at agilent.com/cs/library/usermanuals/public/08052_egfr_pharmdx_interpretation_manu al.pdf.

### Specimen Preparation

Biopsy specimens were handled to preserve the tissue for IHC staining. Standard methods of tissue processing should be used for all specimens. Specimens preserved in the following fixatives are suitable for testing with EGFR pharmDx: 10% (v/v) neutral buffered formalin, 10% (v/v) unbuffered formalin, 25% (v/v) unbuffered formalin, AFA (acetic formalin alcohol), Richard-Allen Scientific's Pen-fix and Bouin's fixative.

### Paraffin-Embedded Sections

Routinely processed and paraffin embedded tissues are suitable for use. Specimens from the biopsy should be blocked into a thickness of 3 or 4 mm and fixed for the time period appropriate to the fixative. The tissues are then dehydrated and cleared in a series of alcohols and xylene, followed by infiltration by melted paraffin. The paraffin temperature should not exceed 60° C. Properly fixed and embedded tissue blocks expressing the EGFR protein will keep indefinitely prior to sectioning and slide mounting if stored in a cool place (15-25° C.).

Tissue specimens should be cut into sections of 3-5 μm. After sectioning, tissues should be mounted on slides and placed in drying racks. The following slides are recommended for use: Fisher's SuperFrost Plus, Dako's Silanized (code S3003), charged or poly-L-lysine coated slides. The slide racks should be pounded on an absorbent towel to remove water trapped under paraffin and on glass and then dried at room temperature for one hour. The rack of slides should then be placed in a 56-60° C. incubator for one hour. Any excess water remaining on slides after removal from the incubator should be removed by pounding slides on towels and drying for one additional hour in the incubator. After removal from the incubator, slides should be held at room temperature until cool and paraffin has hardened. To preserve antigenicity, tissue sections, mounted on slides (Fisher's SuperFrost Plus, poly-L-lysine, charged or Dako's Silanized slides (code S3003), should be stained within 2 months of sectioning when held at room temperature (20-25° C.).

The slides required for EGFR evaluation and verification of tumor presence should be prepared at the same time.

A minimum of 5 slides is recommended, 1 slide for tumor presence, 2 slides for EGFR protein evaluation (one slide for primary antibody and one slide for Negative Control Reagent), and 2 slides for back-up.

### Reagent Preparation

The following reagents are prepared prior to staining: Wash Buffer Solution: Prepare a sufficient quantity of wash buffer by diluting Wash Buffer 10×, 1:10 using distilled or deionized water (reagent-quality water) for the wash steps. Discard buffer if cloudy in appearance.

Substrate-Chromogen Solution (DAB+): This solution should be mixed thoroughly prior to use. Any precipitate developing in the solution does not affect staining quality. To prepare DAB+ Substrate-Chromogen Solution, add 11 drops of Liquid DAB+ Chromogen to one vial of DAB+ Substrate Buffer and mix. Discard any unused solution. Dilute per the guidelines above. Addition of excess Liquid DAB+ Chromogen to the DAB+ Substrate Buffer will result in deterioration of the positive signal.

Counterstain. Prepare ammonia water for counterstain bluing if required. Ammonia water (0.037 mol/L) is prepared by mixing 2.5 (±0.5) mL of 15 mol/L (concentrated) ammonium hydroxide with 1 liter of reagent quality water. Unused 0.037 mol/L ammonia water may be stored at room temperature (20-25° C.) in a tightly capped bottle for up to 12 months.

Mounting Medium. Mounting media such as Dako's Faramount Aqueous Mounting Medium, Ready-to-use (code S3025) or Dako's Glycergel Mounting Medium (code C0563) is recommended for aqueous mounting. Liquify Glycergel by warming to approximately 40(±5) ° C. prior to use. Non-aqueous, permanent mounting is also suitable, such as Dako's Ultramount (code S1964)

### Staining Procedure on the Dako Autostainer

All reagents should be equilibrated to room temperature (20-25° C.) prior to immunostaining. Likewise, all incubations should be performed at room temperature.

Do not allow tissue sections to dry during the staining procedure. Dried tissue sections may display increased nonspecific staining.

Deparaffinization and Rehydration. Prior to staining, tissue slides must be deparaffinized to remove embedding medium and rehydrated. Avoid incomplete removal of paraffin. Residual embedding medium will result in increased nonspecific staining.
STEP 1. Place slides in a xylene bath and incubate for 5 (±1) minutes. Change baths and repeat once.
STEP 2. Tap off excess liquid and place slides in absolute ethanol for 3 (±1) minutes. Change baths and repeat once.
STEP 3. Tap off excess liquid and place slides in 95% ethanol for 3 (±1) minutes. Change baths and repeat once.
STEP 4. Tap off excess liquid and place slides in reagent-quality water for 5 (±1) minutes.
STEP 5. Tap off excess liquid and place slides in Wash Buffer. Begin staining procedure as outlined in Staining Protocol.

Xylene and alcohol solutions should be changed after 40 slides. Toluene or xylene substitutes, such as Histoclear, may be used in place of xylene. EGFR pharmDx includes pretreatment by means of a proteolytic enzyme digestion step. Tissue sections may occasionally be overdigested, causing disruption of cell membranes and overall tissue architecture. Run the assay with careful attention to the duration of the proteolytic digestion step.

### Post-Fixation Procedure

1. Deparaffinize sections and immerse in reagent quality water.
2. Immerse slides in a 10% neutral buffered formalin for 10 minutes.
3. Rinse slides twice in deionized or distilled water.
4. Continue with the EGFR pharmDx staining procedure.

### Automated Staining Protocol

STEP 1. Select desired protocol and program staining run.
STEP 2. Use Auto programs to set up program and begin the EGFR pharmDx program.
STEP 3. Place the reagent vials in the DAKO Autostainer reagent rack according to the computer generated reagent map.
STEP 4. Load the slides onto the DAKO Autostainer according to the computer generated slide map.
STEP 5. Begin the run.
STEP 6. Remove slides from the DAKO Autostainer.

Proceed to Counterstain and Mounting. Rinse slides in reagent-quality water after the DAB+ Substrate-Chromogen solution step. (DAKO Autostainer hardware versions 02 and 03 rinse the slides in reagent-quality water after the substrate-chromogen step. The 01 hardware version of the DAKO Autostainer rinses slides in buffer. Therefore, slides that are stained on 01 hardware must be rinsed with the reagent-quality water after they have been removed from the Autostainer).

### Interpretation of Staining Procedure

Slide evaluation should be performed by a pathologist using a light microscope. All assessments are to be made on the tumor region of the specimen. For evaluation of the immunocytochemical staining and scoring, an objective of 10× or 20× magnification is appropriate.

Use intact cells for interpretation of staining results; necrotic or degenerated cells often stain nonspecifically. Positive and negative cell lines are included in each EGFR pharmDx kit to validate staining runs, every time the assay is performed. Appropriate staining of the control cell lines provides evidence that the EGFR pharmDx assay is functioning properly. No membrane staining of the CAMA-1 control cell line (0) and moderate brown complete or incomplete membrane staining in the HT-29 control cell line (2+) indicates that the staining run is valid. If the staining intensity of the positive control cell line is too weak or too strong a false negative or false positive result may be obtained and the test should be repeated. Reference images are available in the EGFR pharmDx Interpretation Guide.

EGFR pharmDx primarily stains cell membranes, demonstrating both complete and incomplete circumferential staining. The immunostaining pattern is frequently heterogeneous, exhibiting various staining intensities within a single neoplasm.

Staining has also been observed in the cytoplasm and extracellular spaces. Cytoplasmic staining is commonly seen, however the test should be repeated if significant cytoplasmic staining makes it difficult to distinguish membrane staining and interpret the results.

Tumors should be reported as EGFR-positive or EGFR-negative using membrane staining as the evaluable structure. A tumor cell is EGFR-positive if it possesses any membrane staining above background, whether or not it is completely circumferential. A tumor with no membrane staining above background in any tumor cell is reported as an EGFR-negative tumor.

Depending on the incubation length and potency of the hematoxylin used, counterstaining will result in a pale to dark blue coloration of the cell nuclei. Excessive or incomplete counterstaining may compromise interpretation of results.

Staining intensity is established as follows: 3+ (strong staining): visible at low levels of magnification, ×5 objective lens which could be confirmed at higher levels as required; 2+ (moderate staining): visible at intermediate levels of magnification, ×10 or ×20 objective lenses; 1+(weak staining): only reliably confirmable at high magnification, ×40 objective lens; 0 (no staining): no staining visible at high magnification.

Report to treating

| physician | Definition | |
|---|---|---|
| EGFR negative tumor | Absence of membrane staining above background in all | |
| | tumor cells | |
| EGFR positive tumor | EGFR-positive staining is defined as any IHC staining of | |
| | tumor cell membranes above background level; whether it | |
| | is complete or incomplete circumferential staining | |
| | Staining intensity | Percent of tumor cells staining |
| | 1+, 2+ or 3+ | >0% |

### EGFR H-Scoring

Assessment of membranous staining using IHC classifies samples into 4 staining intensity categories (0 to 3+). Of note is that only linear intercellular staining of tumor cells is considered as positive and complete and incomplete membranous staining is considered and recorded. Also, for Histo-score calculation all membrane staining is considered independent of the completeness (complete and incomplete membranous staining).

H-score is assigned using the following formula: [1×(% cells having 1+ staining)+2×(% cells having 2+ staining)+3×(% cells having 3+ staining)] resulting in an H-score for EGFR between 0-300.

### Example 6

p16 status of a sample is determined using the commercially available test kit, based on clone E6H4 (CINtec® Histology, Roche Diagnostics) furthermore using automated slide preparation system, such as BenchMark reader technology, such as the BenchMark XT, the BenchMark ULTRA or the BenchMark GX depending on requested through-put and demand.

Scoring of samples is done following CAP Guideline 2018 by reporting p16 IHC positivity when there is at least 70% nuclear and cytoplasmic expression with at least moderate to strong intensity. A test result is considered indeterminate if the outcome cannot be assessed due to extrinsic factors hampering interpretation. p16 testing can be performed on a sample obtained from a subject suffering from oropharyngeal cancer or on a oropharyngeal cancer/tumor sample.

### Example 7

A 66 years old male patient having been diagnosed with incurable advanced disease in the form of a head and neck cancer, in particular squamous cell carcinoma with a primary tumor location in the larynx, was treated with petosemtamab at 1500 mg Q2W plus pembrolizumab at 400 mg Q6W. The patient tested PD-L1+ with a CPS score of 1 as establish using pharmDx PD-L1 IHC kit using clone 22C3 (Agilent).

Tumor assessment after less than 8 weeks using RECIST 1.1 criteria revealed a partial response (PR) with a 76% shrinkage of target lesions. The PR was confirmed following a second tumor assessment showing 81% shrinkage of target lesions.

### Example 8

A 66 years old female patient having been diagnosed with incurable advanced disease in the form of a head and neck cancer, in particular squamous cell carcinoma with a primary tumor location in the oral cavity, was treated with petosemtamab at 1500 mg Q2W and pembrolizumab at 400 mg Q6W. The patient tested positive for PD-L1 with a CPS score of more than 1 using pharmDx PD-L1 IHC kit using clone 22C3 (Agilent).

Tumor assessment after 7 weeks using RECIST 1.1 criteria revealed a complete response (CR) with a 100% shrinkage of target lesions. The CR was confirmed following second tumor assessment.

### Example 9

A 63 year old male patient having been diagnosed with a head and neck cancer, in particular squamous cell carcinoma with a primary tumor of unknown location, was treated with petosemtamab at 1500 mg Q2W and pembrolizumab at 400 mg Q6W for advanced disease in the neck, lymph nodes, lung and liver. The patient tested PD-L1+ with a CPS score of 43 as establish using pharmDx PD-L1 IHC kit using clone 22C3 (Agilent).

Tumor assessment after less than 8 weeks using RECIST 1.1 criteria revealed a partial response (PR) with a 51% shrinkage of target lesions. The PR was confirmed following a second tumor assessment showing a 62% shrinkage of target lesions.

1. An antibody or functional part, derivative and/or analogue thereof that comprises a variable domain that binds an extracellular part of EGFR for use in the treatment of a cancer in a subject, wherein the treatment further comprises administering an immune checkpoint inhibitor.

2. A method of treating cancer in a subject, the method comprising administering an effective amount of an antibody or functional part, derivative and/or analogue thereof that comprises a variable domain that binds an extracellular part of EGFR and an effective amount of an immune checkpoint inhibitor to the subject.

3. The antibody or functional part, derivative and/or analogue thereof of claim 1, or the method of claim 2, wherein the cancer is an adenocarcinoma, a squamous cell carcinoma, or a head and neck cancer, including squamous cell carcinoma of the head and neck (SCCHN).

4. The antibody or functional part, derivative and/or analogue thereof, or the method of any one of the preceding claims, wherein the cancer is a cancer of the pharynx (including nasopharynx, oropharynx and hypopharynx), oral cavity, larynx, paranasal sinuses, nasal cavity or salivary glands.

5. The antibody or functional part, derivative and/or analogue thereof, or the method of any one of the preceding claims, wherein said immune checkpoint inhibitor comprises a PD-L1, PD-L2 or PD-1 inhibitor.

6. The antibody or functional part, derivative and/or analogue thereof, or the method of any one of the preceding claims, wherein the immune checkpoint inhibitor is an antibody that binds PD-L1, PD-L2 or PD-1.

7. The antibody or functional part, derivative and/or analogue thereof, or the method of any one of the preceding claims, wherein the immune checkpoint inhibitor is pembrolizumab.

8. The antibody or functional part, derivative and/or analogue thereof, or the method of any one of the preceding claims, wherein said subject has not received prior anti-cancer treatment for treatment of said cancer.

9. The antibody or functional part, derivative and/or analogue thereof, or the method of any one of the preceding claims, wherein the subject is a mammal.

10. The antibody or functional part, derivative and/or analogue thereof, or the method of any one of the preceding claims, wherein the subject is a human subject.

11. The antibody or functional part, derivative and/or analogue thereof, or the method of any one of the preceding claims, wherein the antibody or functional part, derivative and/or analogue thereof, is a multispecific antibody.

12. The antibody or functional part, derivative and/or analogue thereof, or the method of any one of the preceding claims, wherein the antibody or functional part, derivative and/or analogue thereof, is a bispecific antibody.

13. The antibody or functional part, derivative and/or analogue thereof, or the use or the method of any one of claims 1-10, wherein the antibody is a monovalent antibody or wherein the antibody comprises said EGFR binding variable domain as the only variable domain.

14. The antibody or functional part, derivative and/or analogue thereof, or the method of any one of claims 1-12, wherein the antibody comprises a variable domain that binds LGR5.

15. The antibody or functional part, derivative and/or analogue thereof, or the method of any one of the preceding claims, wherein the antibody that comprises a variable domain that binds an extracellular part of EGFR, is petosemtamab.

16. The antibody or functional part, derivative and/or analogue thereof, or the method of claim 15, wherein treatment comprises providing a biweekly dose of 1500 mg of the antibody to the subject.

17. The antibody or functional part, derivative and/or analogue thereof, or the method of any one of the preceding claims, wherein said treatment comprises administration of a dose of 400 mg of pembrolizumab as immune check point inhibitor once every six weeks to the subject.

18. The antibody or functional part, derivative and/or analogue thereof, or the method of any one of the preceding claims, wherein petosemtamab as antibody that comprises a variable domain that binds an extracellular part of EGFR and pembrolizumab as immune checkpoint inhibitor are sequentially administered.

19. The antibody or functional part, derivative and/or analogue thereof, or the method of any one of the preceding claims, wherein if pembrolizumab as immune checkpoint inhibitor and petosemtamab as antibody that comprises a variable domain that binds an extracellular part of EGFR are administered on the same day, pembrolizumab is administered after petosemtamab.

20. The antibody or functional part, derivative and/or analogue thereof, or the method of any one of the preceding claims, wherein the antibody or functional part, derivative and/or analogue thereof or the immune checkpoint inhibitor is provided intravenously to the subject.

21. The antibody or functional part, derivative and/or analogue thereof, or the method of any one of the preceding claims, wherein the antibody or functional part, derivative and/or analogue thereof is ADCC enhanced.

22. The antibody or functional part, derivative and/or analogue thereof, or the method of any one of the preceding claims, wherein the antibody or functional part, derivative and/or analogue thereof antibody is afucosylated.

23. The antibody or functional part, derivative and/or analogue thereof, or the method of any one of the preceding claims, wherein the cancer is recurrent, unresectable, locally advanced and/or metastatic cancer.

24. The antibody or functional part, derivative and/or analogue thereof, or the method of any one of the preceding claims, wherein the treatment comprises or is preceded by a step of diagnosing the subject or the cancer for PD-L1 expression.

25. The antibody or functional part, derivative and/or analogue thereof, or the method of claim 24, wherein diagnosing for PD-L1 expression is by immune histochemistry (IHC).

26. The antibody or functional part, derivative and/or analogue thereof, or the method of any one of the preceding claims, wherein the cancer has a Combined Positive Score (CPS) for PD-L1 expression of 1 or more, but not more than 100.

27. A kit of parts comprising an antibody or functional part, derivative and/or analogue thereof that comprises a variable domain that binds an extracellular part of EGFR as defined in any one of the preceding claims, an immune checkpoint inhibitor as defined in any one of the preceding claims and instructions for use of said antibody or functional part, derivative and/or analogue thereof and for use of said immune checkpoint inhibitor.

28. The kit according to claim 27, wherein the antibody is petosemtamab and the instructions comprise instructions for dosing at 1500 mg once every two weeks.

29. The kit according to claim 27 or 28, wherein the immune checkpoint inhibitor is pembrolizumab and instructions for use comprise instructions for dosing at 400 mg once every six weeks.

30. The kit of parts according to any one of claims 27-29, wherein the kit comprises instructions for use of the antibody or functional part, derivative and/or analogue thereof and the immune checkpoint inhibitor in the treatment of head and neck cancer.

31. A combination of an antibody or functional part, derivative and/or analogue thereof that comprises a variable domain that binds an extracellular part of EGFR as defined in any one of claims 1-26 and an immune checkpoint inhibitor as defined in any one of claims 1-26 for use in the treatment of cancer in a subject in need thereof.

32. An immune checkpoint inhibitor, as defined in any one of claims 1-26, for the treatment of cancer in a subject in need thereof, wherein the immune checkpoint inhibitor is for simultaneous or sequential administration with an antibody or functional part, derivative and/or analogue thereof that comprises a variable domain that binds an extracellular part of EGFR as defined in any one of claims 1-26.

33. An antibody or functional part, derivative and/or analogue thereof that comprises a variable domain that binds an extracellular part of EGFR as defined in any one of claims 1-26, for use in the treatment of cancer in a subject to which an immune checkpoint inhibitor, as defined in any one of claims 1-26, has been or will be administered.

34. An immune checkpoint inhibitor, as defined in any one of claims 1-26, for use in the treatment of cancer in a subject to which an antibody or functional part, derivative and/or analogue thereof that comprises a variable domain that binds an extracellular part of EGFR as defined in any one of claims 1-26 has been or will be administered.

35. A combination of an antibody or functional part, derivative and/or analogue thereof that comprises a variable domain that binds an extracellular part of EGFR, as defined in any one of claims 1-26, instructions for use in the treatment of cancer in a subject of said antibody or functional part, derivative and/or analogue thereof, and instructions for use in the treatment of cancer in a subject of an immune checkpoint inhibitor as defined in any one of claims 1-26.

36. A combination of an immune checkpoint inhibitor, as defined in any one of claims 1-26, instructions for use of said inhibitor in the treatment of cancer in a subject and instructions for use in the treatment of cancer in a subject of an antibody or functional part, derivative and/or analogue thereof that comprises a variable domain that binds an extracellular part of EGFR as defined in any one of claims 1-26.

37. A combination of claim 35 or 36, wherein the instructions for use comprise the amount of the immune checkpoint inhibitor and the amount of the antibody or functional part, derivative and/or analogue thereof to be used, the dosing interval and cancer to be treated.
